(19) United States
(12) Patent Application Publication
(10) Pub. No.: US 2005/0250742 A1
(43) Pub. Date: Nov. 10, 2005

(54) Phosphate/sulfate ester compounds and pharmaceutical composition for inhibiting protein interacting NIMA (PIN1)

(75) Inventors: Eleanor Dagostino, San Diego, CA (US); Liming Dong, San Diego, CA (US); Chuangxing Guo, San Diego, CA (US); Xinjun Hou, East Lyme, CT (US); Stephen Margosiak, Escondido, CA (US)

Correspondence Address: AGOURON PHARMACEUTICALS, INC., 10777 SCIENCE CENTER DRIVE, SAN DIEGO, CA, 92121, US

(73) Assignee: Pfizer Inc.

(21) Appl. No.: 10/792,241

(22) Filed: Mar. 3, 2004

(51) Int. Cl. A61K 31/675; C07F 9/6553; C07F 9/6509

(52) U.S. Cl. 514/80; 549/6; 546/22; 544/337; 514/89

(57) ABSTRACT

Phosphate/sulfate ester compounds that modulate and/or inhibit the activity of protein interacting NIMA (PIN1), and to pharmaceutical compositions containing such compounds are described. The invention is also directed to the therapeutic or prophylactic use of such compounds and compositions, and to methods of treating disorders characterized by hypertension, inappropriate cell proliferation, infectious diseases, and neurodegenerative brain disorders, by administering effective amounts of such compounds.

### FIELD OF THE INVENTION

This invention is directed to phosphate/sulfate ester compounds that modulate and/or inhibit the activity of protein interacting NIMA (PIN1), and to pharmaceutical compositions containing such compounds. The invention is also directed to the therapeutic or prophylactic use of such compounds and compositions, and to methods of treating disorders characterized by hypertension, inappropriate cell proliferation, infectious diseases, and neurodegenerative brain disorders, by administering effective amounts of such compounds.

### BACKGROUND OF THE INVENTION

PIN1 is a member of the parvulin family of peptidyl-prolyl isomerases (PPlase) and catalyzes rotation about the peptide bond preceding a proline residue. PIN1 is a regulator of Cdc25, which dephosphorylates Cdc2/cyclinB to drive cells into mitosis.

PIN1 has been identified in all eukaryotic organisms where examined, including plants, yeast, insects, and mammals. The yeast (Ess1) and Dorosophilia (dodo) PIN1 orthologues have high identity to human-expressed sequence tags, which ultimately led to the cloning of the human dodo gene called PIN1. The Dorosophilia dodo gene is reported to be 45% identical to the yeast gene, Ess1.

Using a yeast two-hybrid screen of a human cDNA library, human PIN1 was originally identified as a binding protein of the fungi Aspergillus nidulens protein NIMA. NIMA is a kinase that drives cells into mitosis and is reported to be negatively regulated by PIN1. Depletion of NIMA in A. nidulans cells is reported to lead to cell cycle arrest in G₂ while overexpression is reported to promote premature mitosis. Ser/Thr kinase Cdc2/cyclin B may be the analogous NIMA kinase in human cells although another NIMA-like pathway in human cells is postulated to exist.

Modulation of PIN1 activity is reported to result in dramatic morphological cellular phenotypes. For example, overexpression of PIN1 in Hela cells was reported to cause a G₂ arrest while depletion caused mitotic arrest—the opposite phenotypes observed with NIMA modulation. Additionally, decreasing PIN1 protein expression by full-length antisense expression has been reported to cause cells to progress into mitosis prematurely, to contain aberrant nuclei due to premature chromosome condensation and to induce apoptosis. These data indicate that PIN1 is a negative regulator of mitosis through interactions with a mammalian functional homolog of NIMA and is required for progression through mitosis. Further, depletion of PIN1 is also postulated to play a role in Alzheimers disease. Lu et al., Nature, 380, 544-547 (1996).

In vitro, PIN1 has been reported to interact with mitotic proteins also recognized by the MPM-2 antibody. The MPM-2 monoclonal antibody recognizes a phospho-Ser/Thr-Pro epitope on about approximately 50 proteins associated with mitosis, including important mitotic regulators, such as Cdc25, Wee1, Cdc27, Map 4, and NIMA. See, e.g., Davis et al., Proc. Natl. Acad. Sci. U.S.A. 80, 2926 (1983). PIN1 has also been reported to interact with important upstream regulators of Cdc2/cyclin B, including Cdc25 and its known regulator, Plx1. See Shen et al., Genes Dev. 12, 706 (1998). PIN1, due to its enzymatic action, may remove Cdc25 and Plx1 from play by causing their degradation within the cell.

Studies indicate that the biological function of PIN1 depends on a functional PPlase active site. Lu et al., Science, 283, 1325-1328 (1999). Studies also indicate that PIN1 recognizes its substrates (mitosis-specific phosphoproteins) through the WW domain. The WW domain is a protein recognition motif that is prevalent throughout biology. However, the PIN1 WW domain is unique in that it requires its ligand protein to contain a phosphorylated serine. As with the PPlase domain, a functional WW domain is reported to be essential for biological functions of PIN1. This is consistent with the model where PIN1 recognizes its substrates through the WW domain followed by completion of its essential catalytic role.

Full-length PIN1 protein and the nucleotide sequence encoding full-length PIN1 are disclosed in U.S. Pat. Nos. 5,952,467 and 5,972,697. Additionally, sequences for PIN1 have been deposited in GenBank under accession numbers NM006221 (mRNA) and S68520 (protein). The mRNA sequence for dodo is deposited in GenBank under accession number U35140. Mouse PIN1 mRNA sequence is deposited in GenBank under accession number NM023371.

The crystal structure of full-length PIN1 is reported in Ranganathan, R. et al., Cell, 89, 875-886 (1997) and International Publication No. WO 99/63931. Zhang et al. provide additional analysis of the crystal structure of PIN1 in complex with Ala-Pro (Biochemistry, 41:39 11868-77 (2002)).

Lu et al. (International Publication No. WO 01/38878) and Wulf et al. (EMBO J. 20, 3459-3472 (2001)) disclose that PIN1 is upregulated in human tumors and is a biomarker for cell proliferation.

Inhibitors of PIN1 have been described in the literature. For example, Hennig et al. (Biochemistry, 37, 5953-5960 (1998)) report that juglone (5-hydroxy-1,4-naphthoquinone) selectively inhibits several parvulins, including human PN1. Noel et al. in U.S. Patent Application No. 20010016346, using data based on the crystal structure derived from full-length human PIN1, disclose compounds postulated to be inhibitors of PIN1. Lu et al. in International Publication No. WO 99/12962 report inhibitors that mimic the phospho-Ser/Thr moiety of the phosphoserine or phosphothreonine-proline peptidyl prolyl isomerase substrate.

Given the important role that PIN1 plays in the regulation of the cell cycle, additional compounds that inhibit PIN1 are needed. These compounds, along with pharmaceutical compositions thereof, can serve as effective chemotherapeutic agents for the treatment of a variety of disorders characterized by hypertension, inappropriate cell proliferation, including cancer, infectious diseases, and neurodegenerative brain disorders. The invention provides such compounds that inhibit PIN1.

### SUMMARY OF THE INVENTION

Accordingly, an objective of the invention is to discover compounds and methods for modulating or inhibiting PIN1.

Another objective of the invention is to provide compounds and methods for modulating or inhibiting PIN1 that can be used in pharmaceutical compositions for the treatment of disorders characterized by hypertension, inappropriate cell proliferation, infectious diseases, and neurodegenerative brain disorders.

These and other objectives of the invention, which will become apparent from the following description, have been achieved by the discovery of phosphate/sulfate ester compounds, pharmaceutically acceptable prodrugs, pharmaceutically active metabolites, and pharmaceutically acceptable salts thereof (such compounds, prodrugs, metabolites and salts are collectively referred to as “agents”) described below, which inhibit PIN1. Pharmaceutical compositions containing such agents are useful in treating diseases characterized by hypertension, inappropriate cell proliferation, infectious diseases, and neurodegenerative brain disorders.

In a general aspect, the invention relates to compounds of the Formula I: 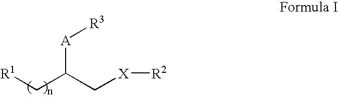
wherein:
n is 1 or 2;
A is a divalent —CH═CH—, —(C₁-C₇-alkyl)-Y—, —NR^{d}(CH₂)ₜ Y—, —Y—(C₁-C₇-alkyl)-, —Y—(C₁-C₇ alkyl)-, —Y—NH—, —Y—NR^{d}(C₁-C₆-alkyl)-, —S—, —S(O)₂—, —O—Y—, —Y—O—, —Y—S—, or —S—Y—, wherein R^{d} is H or C₁-C₆ alkyl, t is an integer from 0 to 5, Y is C(O), C(S), S(O), S(O)₂, or a bond;
X is a direct bond, CH₂, CF₂, O, S, NH, C(O), or C(S);
R¹ is a C₃-C₁₀ cycloalkyl, 4-10 membered heterocycloalkyl, C₆-C₁₀ aryl, or 4-10 membered heteroaryl group, wherein R¹ is unsubstituted or substituted with 1 to 4 R¹⁰ groups;
R² is —S(O)₂OH, —S(O)₂NR^{d}R^{e}, or —P(O)(OR⁴)₂, wherein R⁴ is an H, C₁-C₁₀-alkyl, C₆-C₁₀ aryl, or —CH₂—O—C(O)R^{e}CH₃ group, R^{d} and R^{e} are each independently an H or C₁-C₆ alkyl group, and R⁴ is unsubstituted or substituted with 1 to 4 R¹⁰ groups; and
R³ is OH, C₁-C₇-alkyl, C₁-C₇-alkoxyl, C₆-C₁₀ aryl, 4-10 membered heteroaryl, C₃-C₁₀ cycloalkyl, 3-10 membered heterocycloalkyl, —NH(R⁵), or —N(R⁵)₂ group, wherein R⁵ is independently selected from H, C₁-C₇ alkyl, C₆-C₁₀ aryl, or 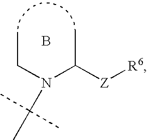
wherein ring B is a 5- or 6-membered heterocycloalkyl group, Z is a divalent C(O)Z′, heteroaryl or heterocycloalkyl group wherein Z′ is a divalent O, S, NH, N(CH₃), CO₂, or CH₂, and R⁶ is H, C₁-C₁₀ alkyl, aryl, C₁-C₆ alkyl-aryl, or arylalkyl group, wherein R³, R⁵, B and R⁶ are unsubstituted or substituted with 1 to 4 R¹⁰ groups;
wherein each R¹⁰ is independently selected from halo, amino, ═O, ═S, ═NH, cyano, nitro, hydroxyl, —SH, haloalkyl, 2-10 membered heteroalkyl, C₁-C₆ alkoxy, C₁-C₁₀ alkyl, C₂-C₆ alkenyl, C₂-C₆ alkynyl, —C(O)ⱼR^{d}, —OC(O)ⱼR^{d}, —OC(O)OC(O)R^{d}, —OOH, C(NR^{d})NR^{b}R^{c}, NR^{d}C(NR^{e})NR^{b}R^{c}, —NR^{d}C(O)ⱼR^{b}, —C(O)NR^{b}R^{c}, —C(O)NR^{d}COR^{b}, —OC(O)NR^{b}R^{c}, —NR^{b}R^{c}, —NR^{d}OR^{c}, —C(S)NR^{b}R^{c}, —NR^{d}C(S)NR^{b}R^{c}, —NR^{d}C(O)NR^{b}R^{c}, —OSH, —S(O)ⱼR^{b}, —OS(O)ⱼR^{b}, —SC(O)R^{b}, —S(O)ⱼC(O)OR^{b}, —SCOR^{d}, NR^{d}SR^{c}, SR^{b}, NHS(O)ⱼR^{b}, COSR^{b}, —C(O)S(O)R^{b}, —CSR^{b}, —CS(O)ⱼR^{b}, —C(SO)OH, —C(SO)₂OH, —NR^{d}C(S)R^{c}, —OC(S)R^{b}, —OC(S)OH, —OC(SO)₂R^{b}, —S(O)ⱼNR^{b}R^{c}, —SNR^{b}R^{c}, —S(O)NR^{b}R^{c}, —NR^{d}CS(O)ⱼR^{c}, —C(O)ⱼ(CH₂)ₜNR^{d}(4-10 membered heteroaryl), —C(O)ⱼ(CH₂)ₜNR^{d}(4-10 membered heterocycloalkyl), —(CR^{d}R^{e})ₜCN, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})C(O)(CR^{d}R^{e})ₜ(C₃-C₁₀ cycloalkyl), —(CR^{d}R^{e})_{q}C(O)(CR^{d}R^{e})ₜ(C₆-C₁₀ aryl), —(CR^{d}R^{e})_{q}C(O)(CR^{d}R^{e})ₜ(4-10 membered heterocycloalkyl), (CR^{d}R^{e})_{q}C(O)(CR^{d}R^{e})ₜ(4-10 membered heteroaryl), —(CR^{d}R^{e})ₜO(CR^{d}R^{e}), (C₃-C₁₀ cycloalkyl), —(CR^{d}R^{e})ₜO(CR^{d}R^{e})_{q}(C₆-C₁₀ aryl), —(CR^{d}R^{e})ₜO(CR^{d}R^{e})_{q}(4-10 membered heterocycloalkyl), —(CR^{d}R^{e})ₜO(CR^{d}R^{e})_{q}(410 membered heteroaryl), —(CR^{d}R^{e})SO₂(CR^{d}R^{e})ₜ(C₃-C₁₀ cycloalkyl), (CR^{d}R^{e})_{q}SO₂(CR^{d}R^{e})ₜ(C₆-C₁₀ aryl), —(CR^{d}R^{e})_{q}SO₂(CR^{d}R^{e})ₜ(4-10 membered heterocycloalkyl), and (CR^{d}R^{e})_{q}SO₂(CR^{d}R^{e})ₜ(4-10 membered heteroaryl), wherein R^{a} is selected from the group consisting of halo, hydroxyl, —NR^{d}R^{e}, C₁-C₁₀ alkyl, haloalkyl, C₁-C₆ alkoxyl, R^{b} and R^{c} are independently selected from H, C₁-C₁₀ alkyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), and —(CR^{d}R^{e})ₜ(4-10 membered heteroaryl), R^{d} and R^{e} are independently H or C₁-C₆ alkyl, j is an integer from 0 to 2, q and t are each independently an integer from 0 to 5, and 1 or 2 ring carbon atoms of the cyclic moieties of the foregoing R¹⁰ groups are unsubstituted or substituted with ═O, and the alkyl, alkenyl, alkynyl, aryl and cyclic moieties of the foregoing R¹⁰ groups are unsubstituted or substituted with 1 to 3 substituents independently selected from halo, ═O, cyano, nitro, —(CR^{d}R^{e})ₜCN, haloalkyl, 2-10 membered heteroalkyl, —OR^{b}, —C(O)ⱼR^{b}, —NR^{d}C(O)R^{b}, —C(O)NR^{b}R^{c}, —NR^{b}R^{c}, —NR^{b}OR^{c}, NR^{d}C(O)ⱼNR^{b}R^{c}, NR^{d}C(O)R^{b}R^{c}, —OC(O)ⱼR^{b}, —OC(O)NR^{b}R^{c}, —SR^{d}, C₁-C₁₀ alkyl, C₂-C₆ alkenyl, C₂-C₆ alkynyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})ₜ(C₆-C₁₀ aryl)-(C₁-C₆ alkyl); wherein t, R^{b}, R^{c}, R^{d}, R^{e} are as defined above.

The invention is also directed to pharmaceutically acceptable prodrugs, pharmaceutically active metabolites, and pharmaceutically acceptable salts of the compounds of Formula I and their pharmaceutically active metabolites. Advantageous methods of making the compounds of the Formula I are also described.

In a preferred embodiment, the invention relates to compounds of Formula I, wherein n is 1 or 2; A is a divalent —NH—Y—, —NR^{d}(CH₂)ₜ—Y—, or —O—Y—, and Y is C(O) or S(O)₂; X is a direct bond, CH₂, O, or S; R¹ is a C₆-C₁₀ aryl or 4-10 membered heteroaryl group unsubstituted or substituted with 1 to 4 R¹⁰ groups; R² is —S(O)₂OH, or —P(O)(OR⁴)₂, wherein R⁴ is an H, C₁-C₁₀ alkyl, or C₆-C₁₀ aryl group, and is unsubstituted or substituted with 1 to 4 R¹⁰ groups; and R³ is a C₆-C₁₀ aryl, 4-10 membered heteroaryl, —NH(C₆H₅), or 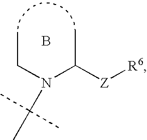
wherein ring B is a 5- or 6-membered heterocycloalkyl group, Z is a divalent C(O)Z′, heteroaryl or heterocycloalkyl group wherein Z′ is a divalent O, S, NH, N(CH₃), CO₂, or CH₂, and R⁶ is H or a C₁-C₁₀ alkyl group, wherein R³, B, and R⁶ is unsubstituted or substituted with 1 to 4 R¹⁰ groups; and wherein R¹⁰ is as defined above.

In a particularly preferred embodiment, the invention relates to compounds of Formula I, wherein n is 1; A is a divalent —NH—Y— or —O—Y—, wherein Y is C(O); X is a direct bond, CH₂, or O; R¹ is a C₆-C₁₀ aryl group unsubstituted or substituted with 1 to 4 R¹⁰ groups; R² is —P(O)(OR⁴)₂, wherein R⁴ is an H, C₁-C₁₀ alkyl, or C₆-C₁₀ aryl group, and is unsubstituted or substituted with 1 to 4 R¹⁰ groups; and R³ is a C₆-C₁₀ aryl, 4-10 membered heteroaryl, or 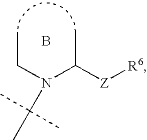
wherein ring B is an unsubstituted 6-membered heterocycloalkyl, Z a divalent C(O)Z′, Z′ is a divalent O, S, or CH₂, and R⁶ is a C₁-C₁₀ alkyl group, wherein R³, B and R⁶ are unsubstituted or substituted with 1 to 4 R¹⁰ groups; and wherein R¹⁰ is as defined above.

In a further particularly preferred embodiment, the invention relates to compounds of Formula I, wherein n is 1; A is —NH—Y— or —O—Y—, wherein Y is C(O); X is a direct bond, CH₂, or 0; R¹ is a C₆-C₁₀ aryl group unsubstituted or substituted with 1 to 4 R¹⁰ groups; R² is —P(O)(OR⁴)₂, wherein R⁴ is an H or a C₁-C₁₀ alkyl group that is unsubstituted or substituted with 1 to 4 R¹⁰ groups; and R³ is a C₆-C₁₀ aryl or 4-10 membered heteroaryl group unsubstituted or substituted with 1 to 4 R¹⁰ groups; and wherein R¹⁰ is as defined above.

Preferably, the invention includes compounds, and pharmaceutically acceptable salts thereof, selected from the following group:

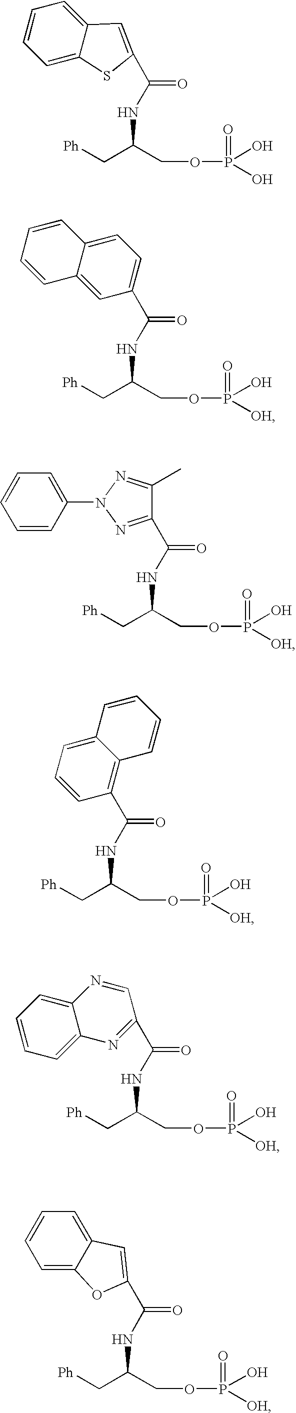

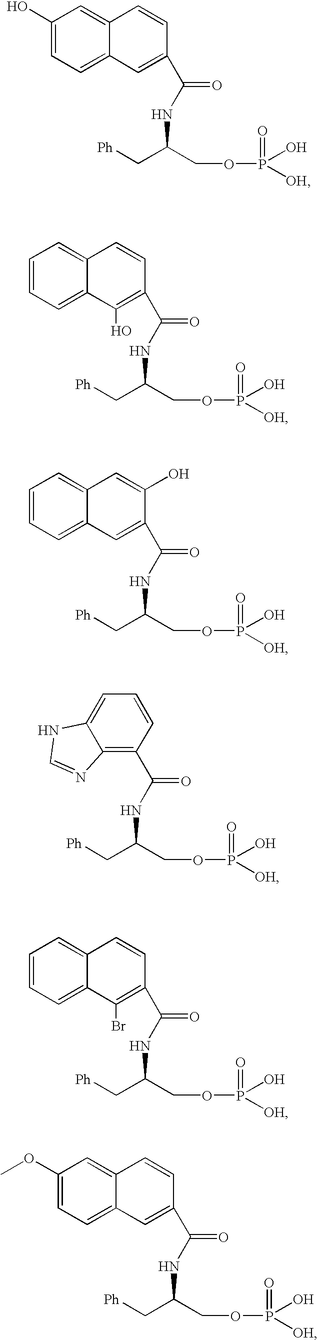

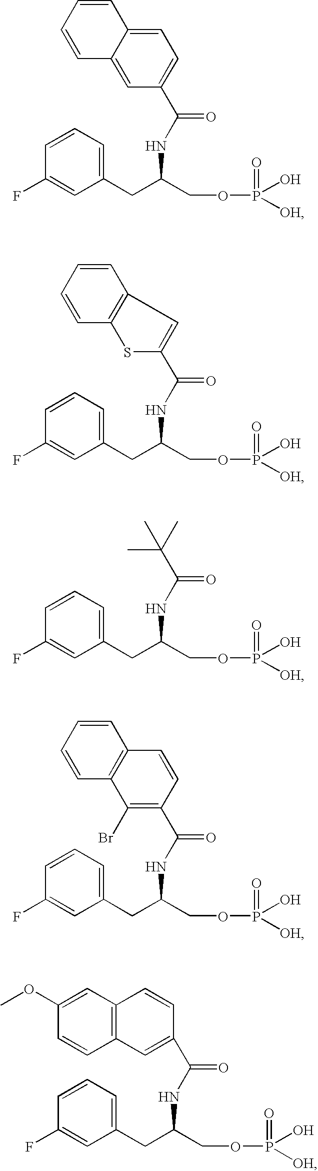

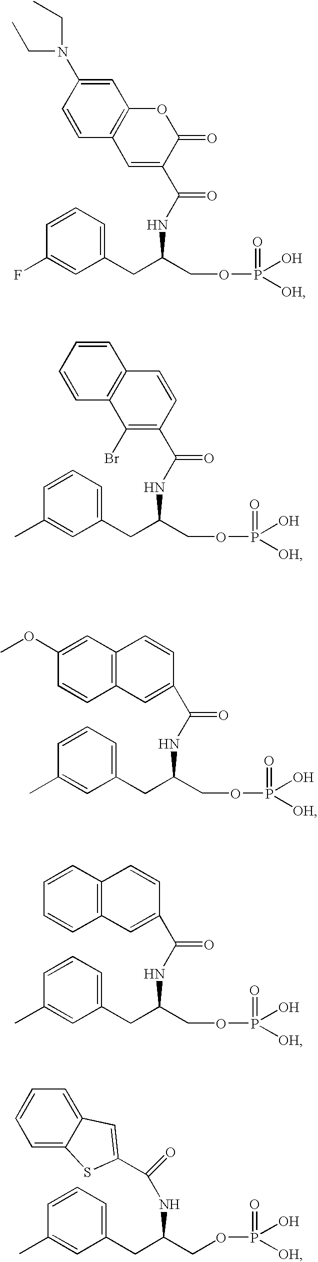

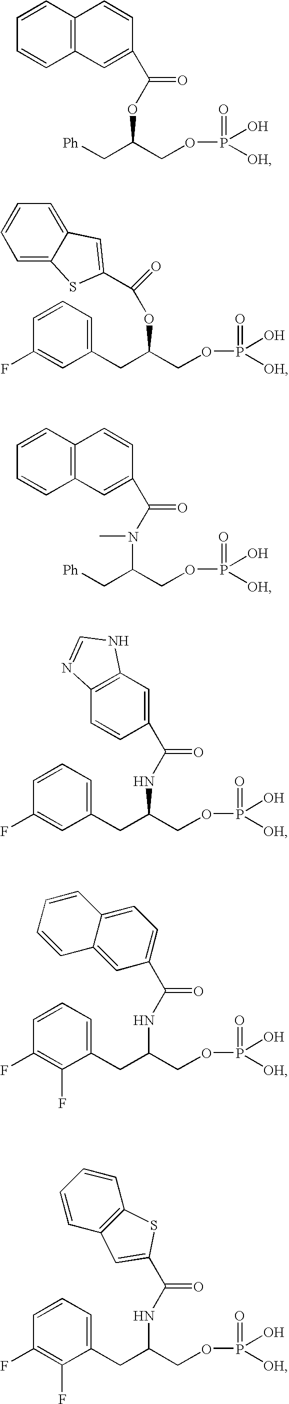

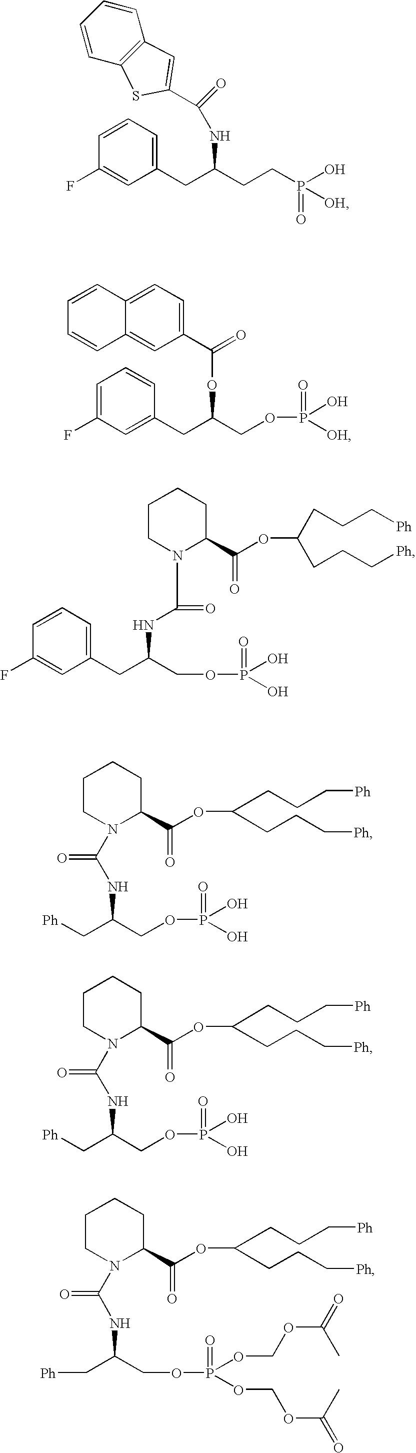

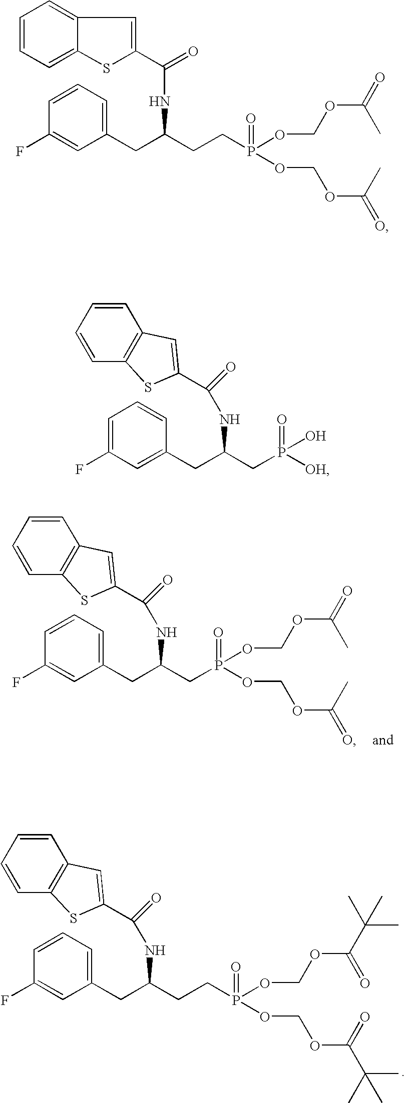

The invention also relates to a method of inhibiting PIN1 by administering a compound of Formula I or a pharmaceutically acceptable prodrug, pharmaceutically active metabolite, or pharmaceutically acceptable salt of such compound or metabolite thereof.

The invention also relates to pharmaceutical compositions, each comprising a therapeutically effective amount of an agent selected from compounds, prodrugs, metabolites, and salts of compounds of Formula I, and a pharmaceutically acceptable carrier or vehicle for such agent. The invention further provides methods of treating mammalian disease conditions mediated by PIN1 activity, by administering to a mammal in need thereof a therapeutically effective amount of a compound, prodrug, active metabolite or salt of a compound of Formula I. The mammalian disease conditions to be treated according to the invention are associated with hypertension, inappropriate cell proliferation (e.g., cancer), infectious diseases (e.g., bacterial and fungal infections), and neurodegenerative brain disorders (e.g., Alzheimer's disease).

The compounds of Formula I are useful for modulating or inhibiting PIN1. More particularly, the compounds are useful as modulating or inhibiting the activity of PIN1, thus providing treatments for hypertension, infectious diseases, neurodegenerative disorders, and cancer or other diseases associated with cellular proliferation.

The terms “comprising” and “including” are used herein in their open, non-limiting sense.

As used herein, “inappropriate cell proliferation” includes diseases or disorders associated with uncontrolled or abnormal cellular proliferation. Such diseases and disorders include, but are not limited to, the following:
a variety of cancers, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers;
a disease process which features abnormal cellular proliferation, e.g., benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections; and
defective apoptosis-associated conditions, such as cancers (including, but not limited to, those types mentioned herein above), viral infections (including, but not limited to, HIV, human papilloma virus, herpesvirus, poxvirus, Epstein-Barr virus, Sindbis virus and adenovirus), prevention of AIDS development in HIV-infected individuals, autoimmune diseases (including, but not limited to, systemic lupus erythematosus, rheumatoid arthritis, psoriasis, autoimmune mediated glomerulonephritis, inflammatory bowel disease and autoimmune diabetes mellitus), neurodegenerative disorders (including, but not limited to, Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including, but not limited to, chronic anemia and aplastic anemia), degenerative diseases of the musculoskeletal system (including, but not limited to, osteroporosis and arthritis), aspirin-sensitive rhinosinusitis, cystic fibrosis, multple sclerosis, kidney diseases, and cancer pain.

The term “alkyl” as used herein refers to a straight- or branched-chain, saturated or partially unsaturated, alkyl group having one to twelve carbon atoms. Preferred alkyl groups have from 1-10, and more preferably from 1-7, carbon atoms. Exemplary alkyl groups include methyl (Me, which also may be structurally depicted by r), ethyl (Et), n-propyl, isopropyl, butyl, isobutyl, sec-butyl, tert-butyl (tBu), pentyl, isopentyl, tert-pentyl, hexyl, isohexyl, and the like. The term ‘lower alkyl’ designates an alkyl having from 1 to 6 carbon atoms (a C₁-C₆alkyl).

The term “aryl” (Ar) refers to a monocyclic, or fused or spiro polycyclic, aromatic carbocycle (ring structure having ring atoms that are all carbon) having from three to twelve ring atoms per ring, preferably 6-10 ring atoms atoms and more preferably 5-7 ring atoms.

Illustrative examples of aryl groups include the following moieties:

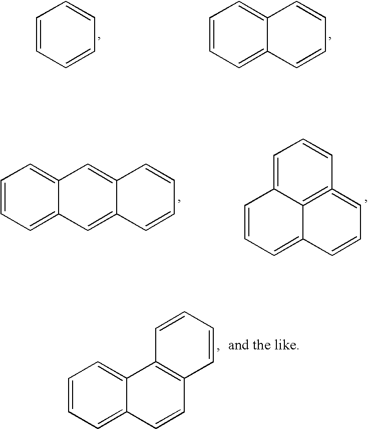

The term “heteroaryl” (heteroAr) refers to a monocyclic, or fused or spiro polycyclic, aromatic heterocycle (ring structure having ring atoms selected from carbon atoms as well as nitrogen, oxygen, and sulfur heteroatoms) having from three to twelve ring atoms per ring, preferably 4-10 ring atoms and more preferably 5-7 ring atoms. Illustrative examples of aryl groups include the following moieties:

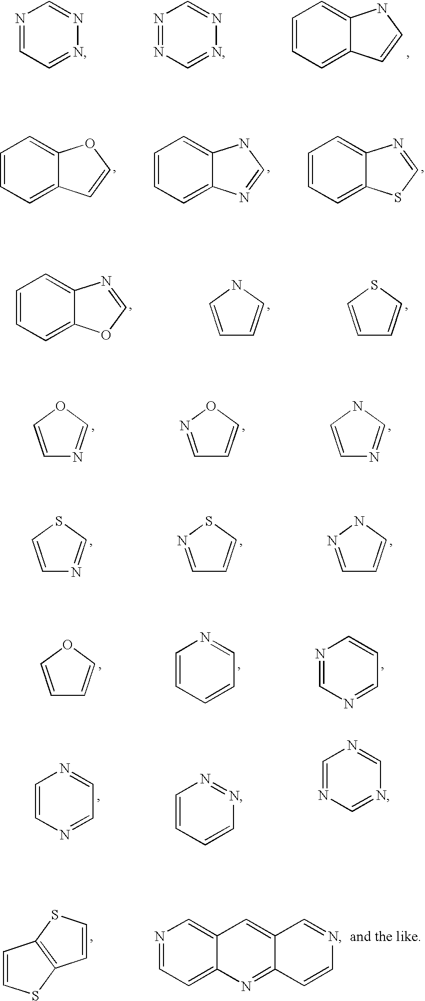

The term ‘cycloalkyl’ refers to a saturated or partially saturated, monocyclic or fused or spiro polycyclic, carbocycle having from three to twelve ring atoms per ring, preferably 3-10 carbon atoms and more preferably 5-7 carbon atoms. Illustrative examples of cycloalkyl groups include the following moieties:

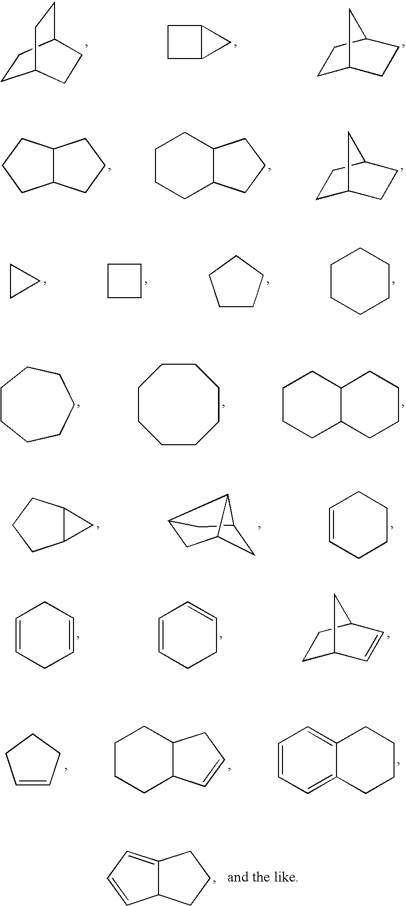

A ‘heterocycloalkyl’ refers to a monocyclic, or fused or spiro polycyclic, ring structure that is saturated or partially saturated and has from three to twelve ring atoms per ring selected from C atoms and N, O, and S heteroatoms, preferably 4-10 ring atoms and more preferably 5-7 ring atoms. Illustrative examples of heterocycloalkyl groups include:

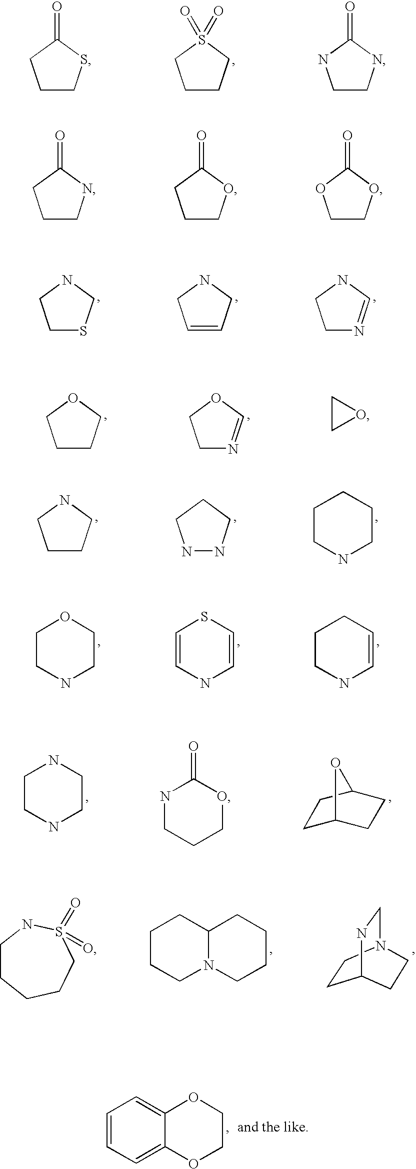

The term ‘alkoxy’ refers to —O-alkyl. Illustrative examples include methoxy, ethoxy, propoxy, and the like.

The term ‘halogen’ represents chlorine, fluorine, bromine or iodine. The term “halo” represents chloro, fluoro, bromo or iodo.

As used herein, ‘haloalkyl’ refers to a loweralkyl radical in which one or more of the hydrogen atoms are replaced by halogen including, but not limited to, chloromethyl, trifluoromethyl, 1-chloro-2-fluoroethyl and the like.

“Heteroalkyl” is an alkyl group (as defined herein) wherein at least one of the carbon atoms is replaced with a heteroatom. Preferred heteroatoms are nitrogen, oxygen, sulfur, and halogen. A heteroatom may, but typically does not, have the same number of valence sites as carbon. Accordingly, when a carbon is replaced with a heteroatom, the number of hydrogens bonded to the heteroatom may need to be increased or decreased to match the number of valence sites of the heteroatom. For instance, if carbon (valence of four) is replaced with nitrogen (valence of three), then one of the hydrogens formerly attached to the replaced carbon must be deleted. Likewise, if carbon is replaced with halogen (valence of one), then three (i.e., all) of the hydrogens formerly bonded to the replaced carbon must be deleted. As another example, trifluoromethyl is a heteroalkyl group wherein the three methyl groups of a t-butyl group are replaced by fluorine. Preferred heteroalkyls of the invention have 2 to 10 member atoms, including both heteroatoms and carbon atoms.

The term ‘substituted’ means that the specified group or moiety bears one or more substituents. The term “unsubstituted” means that the specified group bears no substituents.

The compounds of the invention may exhibit the phenomenon of tautomerism. While Formula I cannot expressly depict all possible tautomeric forms, it is to be understood that Formulas I is intended to represent any tautomeric form of the depicted compound and are not to be limited merely to a specific compound form depicted by the formula drawings.

The compounds of Formula I may have one or more asymmetric centers designated by an asterisk as shown below in Formula I. Additional asymmetric centers may be present on the molecule depending upon the nature of the various substituents on the molecule.

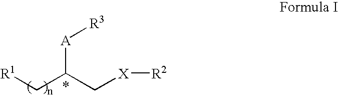

As a consequence of these asymmetric centers, the compounds of Formula I may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

In accordance with a convention used in the art 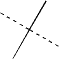
is used in structural formulae herein to depict the bond that is the point of attachment of the moiety or substituent to the core or backbone structure.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomenc excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, Formula I is intended to cover solvated as sell as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, the invention includes pharmaceutically acceptable prodrugs, pharmaceutically active metabolites, and pharmaceutically acceptable salts of such compounds and metabolites.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof.

Prodrugs and active metabolites of a compound may be identified using routine techniques known in the art. See, e.g., Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases; and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

Lu et al. (International Publication No. WO 01/38878; incorporated herein by reference in its entirety) disclose that PIN1 is overexpressed in a variety of cancers, including breast, colon, and prostate. Additionally, the authors disclose that PIN1 is overexpressed in proliferating cells. Therefore, the agents of the invention would have use for treating a variety of cell proliferative diseases associated with overexpression of PIN1.

Therapeutically effective amounts of the agents of the invention may be used to treat diseases mediated by modulation or regulation of PIN1. An ‘effective amount’ is intended to mean that amount of an agent that, when administered to a mammal in need of such treatment, is sufficient to effect treatment for a disease modulated or inhibited by the activity of PIN1. Thus, e.g., a therapeutically effective amount of a compound of the Formula I, salt, active metabolite or prodrug thereof is a quantity sufficient to modulate, regulate, or inhibit the activity of PIN1 such that a disease condition which is mediated by that activity is reduced or alleviated.

The amount of a given agent that will correspond to such an amount wll vary depending upon factors such as the particular compound, disease condition and its severity, the identity (e.g., weight) of the mammal in need of treatment, but can nevertheless be routinely determined by one skilled in the art. The term “treating” refers to:
(i) preventing a disease, disorder, or condition from occurring in an animal that may be predisposed to the disease, disorder and/or condition, but has not yet been diagnosed as having it;
(ii) inhibiting the disease, disorder, or condition, i.e., arresting its development; and
(iii) relieving the disease, disorder, or condition, i.e., causing regression of the disease, disorder, and/or condition.

### DETAILED DESCRIPTION OF THE INVENTION AND PREFERRED EMBODIMENTS

The active agents of the invention may be formulated into pharmaceutical compositions as described below. Pharmaceutical compositions of this invention comprise an effective modulating, regulating, or inhibiting amount of a compound of Formula I and an inert, pharmaceutically acceptable carrier or diluent. In one embodiment of the pharmaceutical compositions, efficacious levels of the inventive agents are provided so as to provide therapeutic benefits involving modulation of PIN1. By “efficacious levels' is meant levels in which the effects of PIN1 activity are, at a minimum, regulated. These compositions are prepared in unit-dosage form appropriate for the mode of administration, e.g., parenteral or oral administration.

An inventive agent can be administered in conventional dosage form prepared by combining a therapeutically effective amount of an agent (e.g., a compound of Formula I) as an active ingredient with appropriate pharmaceutical carriers or diluents according to conventional procedures. These procedures may involve mixing, granulating and compressing or dissolving the ingredients as appropriate to the desired preparation.

The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 060% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease being treated. Optimal dosages for a given set of conditions can be ascertained by those skilled in the art using conventional dosage-determination tests in view of the experimental data for an agent. For oral administration, an exemplary daily dose generally employed is from about 0.001 to about 1000 mg/kg of body weight, with courses of treatment repeated at appropriate intervals. Administration of prodrugs are typically dosed at weight levels, which are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations, which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g, containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The inventive agents may be prepared using the reaction routes and synthesis schemes as described below, employing the general techniques known in the art using starting materials that are readily available. The preparation of preferred compounds of the present invention is described in detail in the following examples, but the artisan will recognize that the chemical reactions described may be readily adapted to prepare a number of other PIN1 inhibitors of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### EXAMPLES

In the examples described below, unless otherwise indicated all temperatures are set forth in degrees Celsius (° C.) and all parts and percentages are by weight. Reagents were purchased from commercial suppliers such as Aldrich Chemical Company or Lancaster Synthesis Ltd. and were used without further purification unless otherwise indicated. Tetrahydrofuran and N,N-dimethylformamide were purchased from Aldrich in Sure Seal bottles and used as received. All solvents were purified using standard methods known to those skilled in the art, unless otherwise indicated.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates from Analtech (0.25 mm), eluted with the appropriate solvent ratios (v/v), and were denoted where appropriate. The reactions were assayed by TLC and terminated as judged by the consumption of starting material.

Visualization of the TLC plates was done with iodine vapor, ultraviolet illumination, 2% Ce(NH₄)₄(SO₄)₄ in 20% aqueous sulfuric acid, or p-anisaldehyde spray reagent, and activated with heat where appropriate. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or Mg₂SO₄ prior to filtration and evaporation of the solvents under reduced pressure on a rotary evaporator and noted as solvents removed in vacuo. Flash column chromatography (Still et al., J. Org. Chem., 43, 2923 (1978)) was done using Merck silica gel (47-61 μm) with a silica gel crude material ratio of about 20:1 to 50:1, unless otherwise stated. Hydrogenolysis was done at the pressure indicated in the examples or at ambient pressure. All melting points (mp) are uncorrected.

¹H-NMR spectra were recorded on a Bruker or Varian instrument operating at 300 MHz and ¹³C-NMR spectra were recorded operating at 75 MHz. NMR spectra were obtained as CDCl₃ solutions (reported in ppm), using chloroform as the reference standard (7.27 ppm and 77.00 ppm) or CD₃OD (3.4 and 4.8 ppm and 49.3 ppm), or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

Infrared (IR) spectra were recorded on a Perkin-Elmer FT-IR Spectrometer as neat oils, as KBr pellets, or as CDCl₃ solutions, and when given are reported in wave numbers (cm⁻¹).

Mass spectrometry (MS) was conducted with various techniques. Mass spectra were obtained using liquid chromatograph electrospray ionization mass spectrometry, MS (ESP). Matrix-Assisted Laser Desorption/Ionization (MALDI) Fourier Transform Mass Spectrometry was performed on an IonSpec FTMS mass spectrometer.

The following compounds of the invention were made according to the general synthetic pathways shown in Schemes 1-10 and the detailed experimental procedures that follow thereof. These synthetic pathways and experimental procedures utilize many common chemical abbreviations, such as THF (tetrahydrofuran), DMF (N,N-dimethylformamide), EtOAc (ethyl acetate), DBU (1,8-diazacyclo[5.4.0]undec-7-ene), TMSCI (trimethylsilyl chloride), MCPBA (3-chloroperoxybenzoic acid), EDC (1-(3-dimethylaminopropyl)-3-ethylcarbodiimide hydrochloride), HOBT (1-hydroxybenzotriazole hydrate), DMAP (4-dimethylaminopyridine), TBDPSCI (t-butyldiphenylchlorosilane), TMSBr (bromotrimethylsilane), DIEA (diisopropylethylamine), TBAI (tetrabutylamonium iodide), and the like. 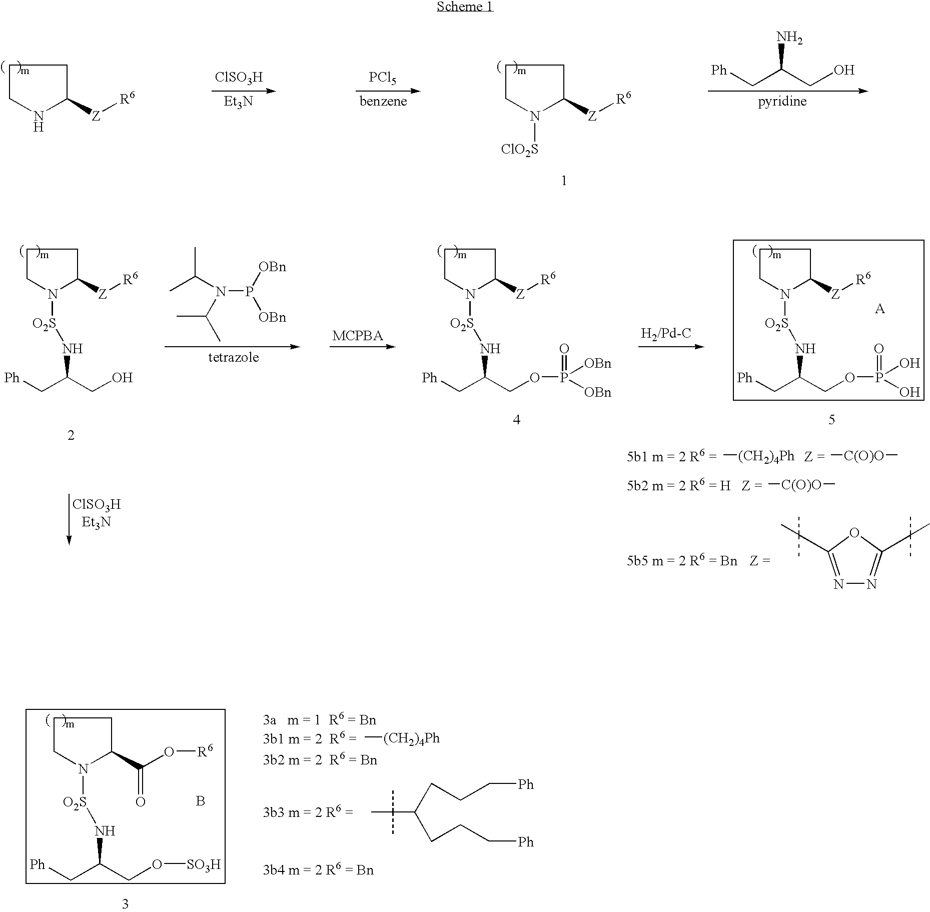
Alcohol 2a:

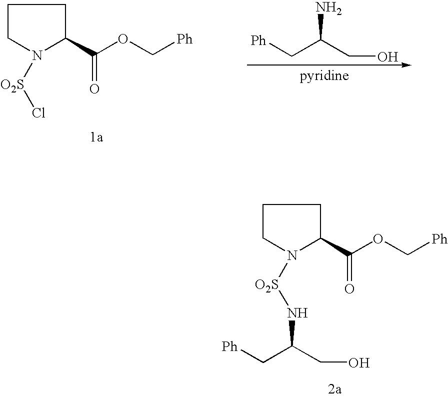

To a pyridine solution (1 mL) of the sulfamoyl chloride 1a (0.14 g, 0.5 mmol, preparation described in International Publication No. WO 0140185) was added D-phenylalaninol (0.151 g, 1 mmol) at 25° C. After 12 h, the reaction mixture was concentrated in vacuo. The yellow residue was purified by flash column chromatography (25% ethyl acetate (EtOAc) in hexanes) followed by preparative TLC to afford 70 mg (33% yield) of the compound 2a. ¹H NMR (CDCl₃): δ 7.24-7.0 (10H, m), 4.89 (2H, AB), 4.70 (1H, d, J=8.7 Hz), 4.25 (1H, dd, J=8.7, 3.6 Hz), 3.54 (2H, m), 3.28 (1H, m), 3.04 (2H, m), 2.66 (2H, d, J=6.6 Hz), 2.33 (1H, m), 2.01 (1H, m); HRMS (FAB) calc for C₂₁H₂₇N₂O₅S (M+H⁺) 419.1641; found 419.1629.

### Example 3a

### 1-(2-Phenyl-1-sulfooxymethyl-othylsulfamoyl)-pyrrolidine-2S-carboxylic acid benzyl ester

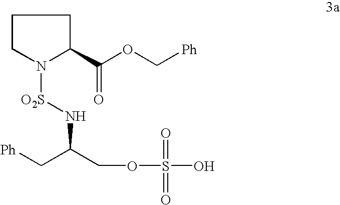

At −70° C., a methylene chloride solution (2 mL) of the alcohol 2a (10 mg, 0.024 mmol) was added triethylamine (Et₃N, 0.05 mL) and chlorosulfonic acid (8 mg, 5 μl, 0.068 mmol). The cooling bath was then removed and the reaction mixture was allowed to warm to 25° C. over 3 h. All solvent was evaporated in vacuo. The residue was purified by column chromatography (3% methanol (MeOH) in EtOAc) to give 8 mg (67% yield) of the title compound 3a. ¹H NMR (CD₃OD): δ 7.4-7.15 (10H, m), 5.16 (2H, AB), 4.22 (1H, dd, J=8.7, 3.9 Hz), 4.04 (1H, dd, J=10.2, 4.8 Hz), 3.91 (1H, dd, J=10.2, 4.5 Hz), 3.77 (1H, m), 3.38 (1H, m), 3.14 (1H, m), 2.93 (1H, dd, J=14.1, 9.1 Hz), 2.81 (1H, dd, J=14.1, 6.6 Hz), 2.18 (1H, m), 1.97-1.73 (3H, m); MS (ESP): 497 (M−H+); HRMS (FAB) calc for C₂₁H₂₆N₂O₈S₂Na (M+Na⁺) 521.1028; found 521.1010.
Alcohol 2b1:

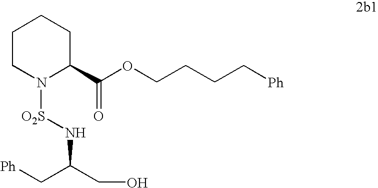

Prepared as described in the synthesis of 2a using the n-butyl-phenyl ester of sulfamoyl chloride 1a (0.15 g, 0.42 mmol, preparation described in International Publication No. WO 0140185) and D-phenylalaninol (0.18 g, 1.2 mmol). After purification by flash column chromatography (25% EtOAc in hexanes), the compound 2b1 (62 mg) was obtained in 31% yield. ¹H NMR (CDCl₃): δ 7.4-7.1 (10H, m), 5.17 (1H, d, J=8.1 Hz), 4.68 (1H, d, J=4.5 Hz), 4.20 (2H, m), 3.75 (2H, m), 3.55 (1H, m), 3.35 (1H, br d, J=12.1 Hz), 2.95-2.73 (3H, m), 2.67 (2H, m), 2.45 (1H, br s), 2.24 (1H, d, J=13.1 Hz); HRMS (FAB) calc for C₂₅H₃₄N₂O₅SNa (M+Na⁺) 497.2086; found 497.2079.

### Example 3b1

### 1-(2-Phenyl-1-sulfooxymethyl-ethylsulfamoyl)-piperidine-2S-carboxylic acid 4-phenyl-butyl ester

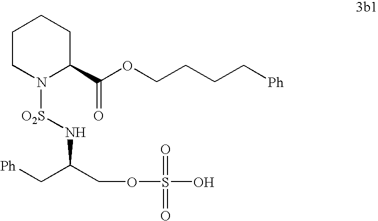

Prepared as described in the synthesis of 3a using the alcohol 2b1 (10 mg, 0.021 mmol), chlorosulfonic acid (6 mg, 4 μl, 0.055 mmol) and triethylamine (0.015 mL). The reaction mixture was diluted with EtOAc (20 mL) and washed with ice-cold 5% hydrochloric (HCl) solution (1×20 mL). Column chromatography (8% MeOH in EtOAc) afforded 10 mg (85% yield) of the title compound 3b1. ¹H NMR (CDCl₃): δ 7.3-6.9 (10H, m), 6.03 (1H, br s), 4.26 (2H, m), 4.05 (2H, m), 3.93 (1H, br s), 3.58 (1H, br s), 3.13 (1H, m), 2.79 (3H, m), 2.48 (2H, m), 2.30 (1H, m), 1.88 (1H, m); HRMS (FAB) calc for C₂₅H₃₃N₂O₈S₂CS₂ (M−H⁺+2Cs⁺) 818.9787; found 818.9756.
Alcohol 2b2:

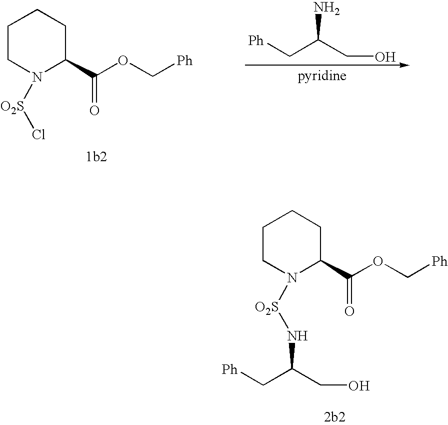

Prepared as described in the synthesis of 2a using the sulfamoyl chloride 1b2 (0.15g, 0.47 mmol, preparation described in International Publication No. WO 0140185) and D-phenylalaninol (0.214 g, 1.4 mmol). After purification by flash column chromatography (25% to 30% EtOAc in hexanes), the compound 2b2 (66 mg) was obtained in 33% yield. ¹H NMR (CDCl₃): δ 7.4-7.17 (10H, m), 5.19 (2H, AB), 5.03 (1H, d, J=8.4 Hz), 4.71 (1H, br d, J=4.8 Hz), 3.8-3.6 (2H, m), 3.48 (1H, m), 3.31 (1H, brd, J=12.6 Hz), 2.88-2.74 (3H, m), 2.34 (1H, brt, J=5.1 Hz), 2.25 (1H, m).

### Example 3b2

### 1-(1-Benzyl-2-sulfooxy-ethylsulfamoyl)-piperidine-2S-carboxylic acid benzyl ester

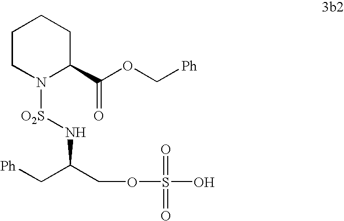

Prepared as described in the synthesis of 3a using the alcohol 2b2 (30 mg, 0.069 mmol), chlorosulfonic acid (16 mg, 10 μl, 0.13 mmol) and triethylamine (0.05 mL). The reaction mixture was diluted with EtOAc (20 mL) and washed with ice-cold 5% HCl solution (1×20 mL). Column chromatography purification (5% MeOH in EtOAc) afforded 25 mg (70% yield) of the title compound 3b2. ¹H NMR (CDCl₃): δ 7.31-7.01 (10H, m), 6.03 (1H, br d, J=9 Hz), 5.16 (1H, d, J=12.3 Hz), 5.00 (1H, d, J=12.3 Hz), 4.43-4.30 (2H, m), 4.1 (1H, m), 3.66 (1H, m), 3.37 (3H, m), 3.14 (1H, m), 2.80 (3H, m), 1.98 (1H, br d); HRMS (FAB) calc for C₂₂H₂₇N₂O₈S₂Na₂ (M−H⁺+2Na⁺) 557.1004; found 557.1019.
Alcohol 2b3:

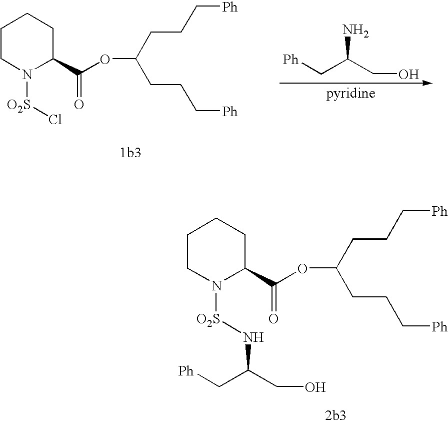

Prepared as described in the synthesis of 2a using the sulfamoyl chloride 1b3 (0.05 g, 0.105 mmol, preparation described in International Publication No. WO 0140185) and D-phenylalaninol (0.026 g, 0.17 mmol). After purification by flash column chromatography (25% to 30% EtOAc in hexanes), the compound 2b3 (30 mg) was obtained in 48% yield. ¹H NMR (CDCl₃): δ 7.35-7.09 (15H, m), 5.07 (1H, d, J=7.8 Hz), 5.02 (1H, m), 4.64 (1H, br d, J=4.5 Hz), 3.69 (2H, m), 3.47 (1H, m), 3.30 (1H, br d, J=12.6 Hz), 2.84 (2H, d, J=7.2 Hz), 2.80 (1H, td, J=13.5, 3.9 Hz), 2.67-2.51 (4H, m), 2.26-2.08 (2H, m).

### Example 3b3

### 1-(2-Phenyl-1-sulfooxymethyl-ethylsulfamoyl)-piperidine-2S-carboxylic acid 4-phenyl-1-(3-phenyl-propyl)-butyl ester

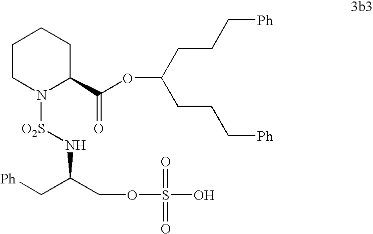

Prepared as described in the synthesis of 3a using the alcohol 2b3 (10 mg, 0.0169 mmol), chlorosulfonic acid (8 mg, 5 μl, 0.068 mmol) and triethylamine (0.05 mL). The reaction mixture was diluted with EtOAc (20 mL) and washed with ice-cold 5% HCl solution (1×20 mL). Column chromatography purification (5% MeOH in EtOAc) afforded 8 mg (70% yield) of the title compound 3b3. ¹H NMR (CDCl₃): δ 7.36-7.00 (15H, m), 6.06 (1H, br d, J=7.5 Hz), 4.90 (1H, m), 4.31 (1H, br d), 4.20 (1H, br s), 4.06 (1H, m), 3.66 (1H, m), 3.34 (1H, br d, J=11.1 Hz), 2.85 (3H, m), 2.51 (4H, m); HRMS (FAB) calc for C₃₄H₄₃N₂O₈S₂Cs₂ (M−H⁺+2Cs⁺) 937.0570; found 937.0557.

### Synthesis of Benzyl Ester

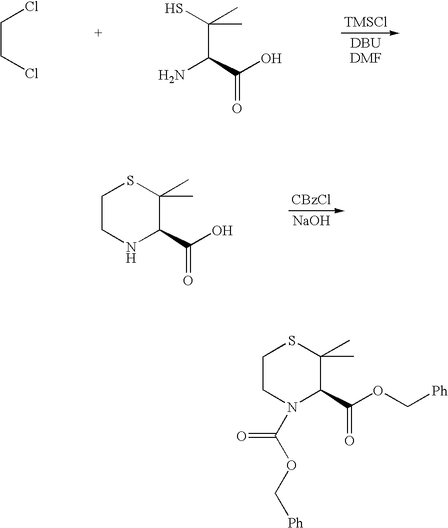

To a mixture of L-penicillamine (14.92 g), 1,2-dichloroethane (300 mL) and DMF (2 mL) at 0° C. was added 1,8-diazacyclo[5.4.0]undec-7-ene (DBU, 22.4 mL), followed by trimethylsilyl chloride (TMSCI, 19 mL). After stirring for 3 h, the solution was warmed to 25° C., followed by the slow addition of DBU (29.9 mL). The reaction mixture was stirred for 17 h at 25° C. Methanol (10 mL) was added and a precipitate formed. The precipitate was collected by filtration and was rinsed with a minimum amount of methanol. The solid was dried in vacuo at 50° C. for 6 h to give 3(R)-2,2-dimethyl-tetrahydro-2H-1,4-thiazine-3-carboxylic acid as a white powder (16 g). At 0° C., a portion of the thiazine (0.4 g, 2.3 mmol) was dissolved in a NaOH solution (1 N, 12 mL). To the resulting mixture was added benzylchloroformate (1.4 mL, 9.2 mmol). After 15 h at 25° C., the solution was diluted with water (20 mL) and extracted with EtOAc (2×25 mL). The extracts were dried (MgSO₄) and concentrated in vacuo. Flash column chromatography (15-20% EtOAc in hexanes) purification afforded 0.63 g of the title compound 3b3. ¹H NMR (CDCl₃): (mixture of two rotamers) δ 7.3 (10H, m), 7.11 (4H, br s), 4.87 and 4.70 (1H, s), 4.40 and 4.28 (1H, d, J=6.7 Hz), 3.72 and 3.60 (1H, m), 2.94 (1H, m), 2.37 (1H, t, J=3.9 Hz), 1.45 (3H, s), 1.34 (3H, s); MS (ESP) 400 (M+H⁺).
Sulfamoyl Chloride 1b4:

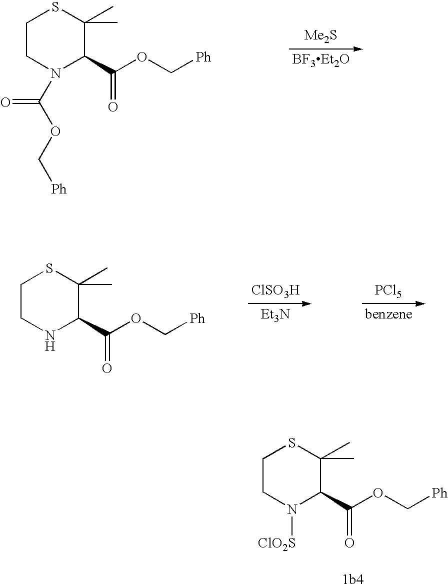

To a methylene chloride solution (2 mL) of the benzyl ester (0.6 g, 1.5 mmol) at 0° C. was added methyl sulfide (1 mL) and BF₃•Et₂O (0.2 mL). After 16 h, the reaction mixture was added sat'd NaHCO₃ solution (5 mL) and extracted with methylene chloride (2×20 mL). Combined organic layers were washed with brine (1×25 mL) and dried (Na₂SO₄). All solvent was removed in vacuo to give a pale yellow oil (0.35 g), which was dissolved in methylene chloride (8 mL). To the resulting solution was slowly added triethylamine (1 mL) and ClSO₃H (0.23 g, 1.97 mmol). The mixture was allowed to warm to about 25° C. and stirred at that temperature for about 2 h. The solution was then concentrated in vacuo after which benzene (2×15 mL) was added and evaporated to remove trace amounts of Et₃N and water. To the residue was added benzene (20 mL) and PCl₅ (0.41 g, 1.97 mmol). The suspension was heated at reflux for about 30 minutes, then cooled to about 25° C. and poured into an ice-cold NaOH solution (5%, 40 mL). The aqueous mixture was extracted with CH₂Cl₂ (3×30 mL), dried over sodium sulfate and concentrated. The residue was purified by flash column chromatography (3% EtOAc in hexanes) affording 0.355 g (74%) of the compound 1b4. Amine: ¹H NMR (CDCl₃): δ 7.4-7.3 (5H, m), 5.14 (2H, AB), 3.75 (1H, s), 3.38 (1H, m), 2.92 (2H, m), 2.27 (1H, m), 1.40 (3H, s), 1.26 (3H, s); Sulfamoyl Chloride 1b4: ¹H NMR (CDCl₃): δ 7.43-7.29 (5H, m), 5.22 (2H, AB), 4.53 (1H, s), 4.24-4.05 (2H, m), 3.17 (1H, dd, J=11.7, 4.5 Hz), 3.13 (1H, dd, J=11.7, 4.8 Hz), 2.56 (1H, dt, J=14.1, 2.7 Hz), 1.58 (3H, s), 1.29 (3H, s).
Alcohol 2b4:

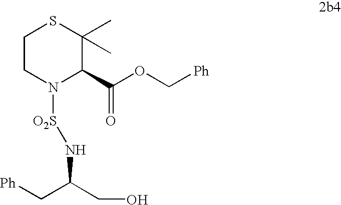

Prepared as described in the synthesis of 2a using the sulfamoyl chloride 1b4 (0.2 g, 0.55 mmol) and D-phenylalaninol (0.166 g, 1.1 mmol). After purification by flash column chromatography (25% to 30% EtOAc in hexanes), the compound 2b4 (10 mg) was obtained in 4% yield. ¹H NMR (CDCl₃): δ 7.42-7.12 (10H, m), 5.18 (2H, AB), 4.46 (2H, m), 3.68-3.59 (3H, m), 3.48 (1H, m), 3.39 (1H, m), 2.95 (1H, m), 2.76 (2H, d, J=7.2 Hz), 2.32 (1H, dt, J=13.5, 2.1 Hz), 1.87 (3H, s), 1.34 (3H, s).

### Example 3b4

### 3,3-Dimethyl-1-(2-phenyl-1-sulfooxymethyl-ethylsulfamoyl)-piperidine-2R-carboxylic acid benzyl ester

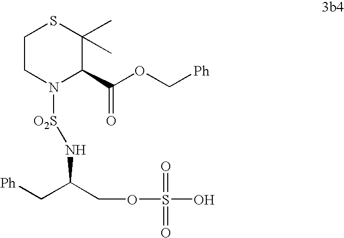

Prepared as described in the synthesis of 3a using the alcohol 2b4 (6 mg, 0.0126 mmol), chlorosulfonic acid (6 mg, 4 μl, 0.04 mmol) and triethylamine (0.04 mL). The reaction mixture was diluted with EtOAc (20 mL) and washed with ice-cold 5% HCl solution (1×20 mL). Column chromatography purification (5% MeOH in EtOAc) afforded 3 mg (43% yield) of the title compound 3b4. ¹H NMR (CDCl₃): δ 7.34-7.06 (10H, m), 5.04 (2H, s), 4.23 (1H, s), 3.89 (1H, dd, J=9.9, 4.2 Hz), 3.82 (1H, dd, J=9.9, 5.1 Hz), 3.59 (1H, td, J=12.9, 3 Hz), 3.52-3.33 (2H, m), 2.88 (1H, dd, J=14.1, 6.8 Hz), 2.64 (1H, dd, J=14.1, 7.5 Hz), 2.53 (1H, td, J=12.8, 4.2 Hz), 2.13 (1H, dt, J=14.1, 2.4 Hz), 1.37 (3H, s), 1.09 (3H, s); MS (ESP) 557 (M−H).
Phosphate Benzyl Ester 4b1:

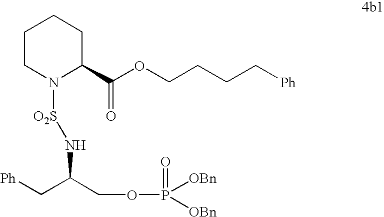

To an acetonitrile solution (7 mL) of the alcohol 2b1 (25 mg, 0.0527 mmol) and 1H-tetrazole (7.4 mg, 0.105 mmol) was added dibenzyl N,N-diisopropylphosphoramidite (27.3 mg, 0.079 mmol) at 25° C. After 1 h, 3-chloroperoxybenzoic acid (MCPBA, 34 mg, 70% pure, 0.139 mmol) was added to the suspension. The solution was diluted with ether (40 mL), washed with concentrated NaHSO₃ solution (2×30 mL), dried over MgSO₄ and concentrated in vacuo. The residue was purified by preparative TLC to give 29.5 mg of the compound 4b1 in 76% yield. ¹H NMR (CDCl₃): δ 7.30-7.05 (20H, m), 5.09 (1H, d, J=9.3 Hz), 4.99 (4H, m), 4.52 (1H, m), 4.14-3.83 (4H, m), 3.68 (1H, m), 3.20 (1H, d, J=12.9 Hz), 2.75 (2H, d, J=7.2 Hz), 2.72 (1H, m), 2.54 (2H, m), 2.10 (1H, d, J=13.5 Hz); HRMS (FAB) calc for C₃₉H₄₇N₂O₈PSCs (M+Cs⁺) 867.1845; found 867.1868.

### Example 5b1

### 1-(2-Phenyl-1-phosphonooxymethyl-ethylsulfamoyl)-piperidine-2S-carboxylic acid 4-phenyl-butyl ester

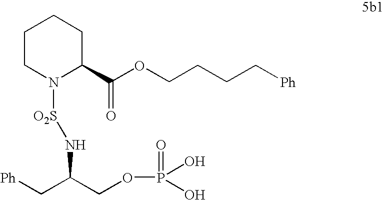

To a methanol solution of the phosphate benzyl ester 4b1 (29.5 mg, 0.0402 mmol) was added palladium on carbon (10%, 5 mg). The suspension was kept under hydrogen (1 atm) for 1.5 h. After filtration, the filtrate was concentrated to dryness, affording 22.6 mg of the title compound 5b1 in 100% yield. ¹H NMR (CDCl₃): δ 7.34-6.95 (10H, m), 4.47 (1H, s), 4.17-3.82 (4H, m), 3.62 (1H, br s), 3.20 (1H, br d), 3.28 (3H, m), 2.53 (2H, m), 1.21 (1H, br d); HRMS (FAB) calc for C₂₅H₃₅N₂O₈PSNa (M+Na⁺) 577.1749; found 577.1769.
Phosphate Benzyl Ester 4b2:

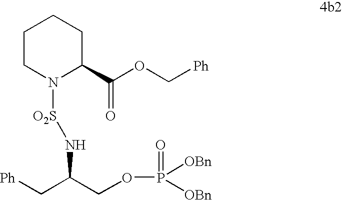

Prepared as described in synthesis of 4b1 using the alcohol 2b2 (240 mg, 0.554 mmol), 1H-tetrazole (77 mg, 1.11 mmol), dibenzyl N,N-diisopropylphosphoramidite (249 mg, 0.72 mmol). Hydrogen peroxide (30%, 2 mL) instead of MCPBA was used for the oxidation. Column chromatography purification (30% EtOAc in hexanes) provided 300 mg of the compound 4b2 in 83% yield. ¹H NMR (CDCl₃): δ 7.41-7.10 (20H, m), 5.11-5.00 (6H, m), 4.66 (1H, J=4.5 Hz), 4.07 (1H, m), 3.89 (1H, m), 3.72 (1H, m), 3.26 (1H, br d, J=13.2 Hz), 2.85-2.71 (3H, m), 2.22 (1H, d, J=12.9 Hz).

### Example 5b2

### 1-(2-Phenyl-1-phosphonooxymethyl-ethylsulfamoyl)-piperidine-2-carboxylic acid

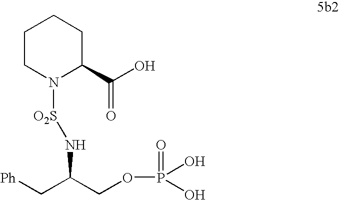

Prepared as described in the synthesis of 5b1 from phosphate benzyl ester 4b2 (300 mg, 0.457 mmol). The benzyl ester of the carboxylate was also cleaved to carboxylic acid during the hydrogenation. The title compound 5b2 was obtained in 68% yield (119 mg). ¹H NMR (CD₃OD): δ 7.37-7.13 (5H, m), 4.46 (1H, d, J=2.1 Hz), 4.05 (2H, m), 3.64 (1H, m), 3.28 (1H, m), 3.05 (1H, m), 3.00 (1H, dd, J=13.8, 6.8 Hz), 2.82 (1H, dd, J=13.8, 7.5 Hz), 2.15 (1H, d, J=12.9 Hz); LCMS: 423 (M+H⁺); HRMS (FAB) calc for C₁₅H₂₄N₂O₈PS (M+H⁺) 423.0991; found 423.0995.
Alcohol 2b5:

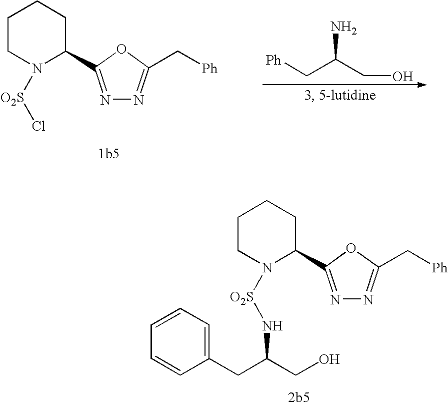

Prepared as described in the synthesis of 2a using the sulfamoyl chloride 1b5 (0.07 g, 0.205 mmol, preparation described in International Publication No. WO 0140185) and D-phenylalaninol (0.1 g, 0.662 mmol). 3,5-Lutidine was employed as the reaction solvent in place of pyridine. After purification by flash column chromatography (50% EtOAc in hexanes), the compound 2b5 (28 mg) was obtained in 30% yield. ¹H NMR (CDCl₃): δ 7.43-7.13 (10H, m), 5.53 (1H, d, J=8.4 Hz), 5.27 (1H, br d, J=3.3 Hz), 4.19 (2H, AB), 3.71 (2H, m), 3.48 (1H, m), 3.35 (1H, dt, J=12.9, 3.3 Hz), 2.91-2.74 (3H, m), 2.53 (1H, t, J=6.3 Hz), 2.19 (1H, dq, J=13.5, 3 Hz), 1.94 (1H, tdd, J=13.6, 5.3, 3.8 Hz).
Benzyl Phosphate Ester 4b5:

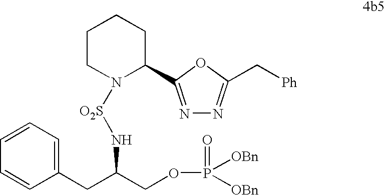

Prepared as described in synthesis of 4b1 using the alcohol 2b5 (28 mg, 0.061 mmol), 1H-tetrazole (8 mg, 0.12 mmol), dibenzyl N,N-diisopropylphosphoramidite (25 mg, 0.072 mmol) and MCPBA (33 mg, 70% pure, 0.13 mmol). Column chromatography purification (30 to 60% EtOAc in hexanes) followed by preparative TLC purification (50% EtOAc in hexanes) provided 30 mg of the compound 4b5 in 69% yield. ¹H NMR (CDCl₃): δ 7.44-7.07 (20H, m), 5.57 (1H, d, J=8.4 Hz), 5.19 (1H, br d, J=3 Hz), 5.13-4.97 (4H, m), 4.13 (2H, AB), 4.04 (1H, m), 3.91 (1H, m), 3.75 (1H, m), 3.26 (1H, dt, J=12.6, 3 Hz), 2.89-2.59 (3H, m), 2.15 (1H, dq, J=13.8, 3 Hz), 1.89 (1H, m), 1.74 (1H, br s), 1.64 (1H, dt, J=13.2, 3.3 Hz).

### Example 5b5

### Phosphoric acid mono-{(R)-2-[(S)-2-(5-benzyl-[1,3,4]oxadiazol-2-yl)-piperidine-1-sulfonylamino]-3-phenyl-propyl}ester

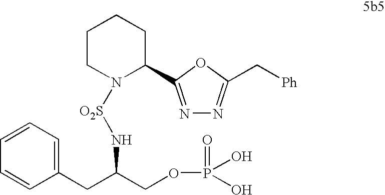

Prepared as described in the synthesis of 5b1 using phosphate benzyl ester 4b5 (30 mg, 0.042 mmol) and 10% palladium on carbon (5 mg). The title compound 5b5 was obtained in quantitative yield (28 mg). ¹H NMR (CD₃OD): δ 7.32-7.01 (10H, m), 4.95 (1H, m), 4.12 (2H, AB), 3.92-3.76 (2H, m), 3.51 (1H, m), 3.24 (1H, br d), 2.93-2.76 (2H, m), 2.67 (1H, dd, J=13.8, 7.8 Hz), 1.92 (1H, br d), 1.79 (1H, m); MS (ESP): 559 (M+Na⁺); 535 (M−H)⁻. 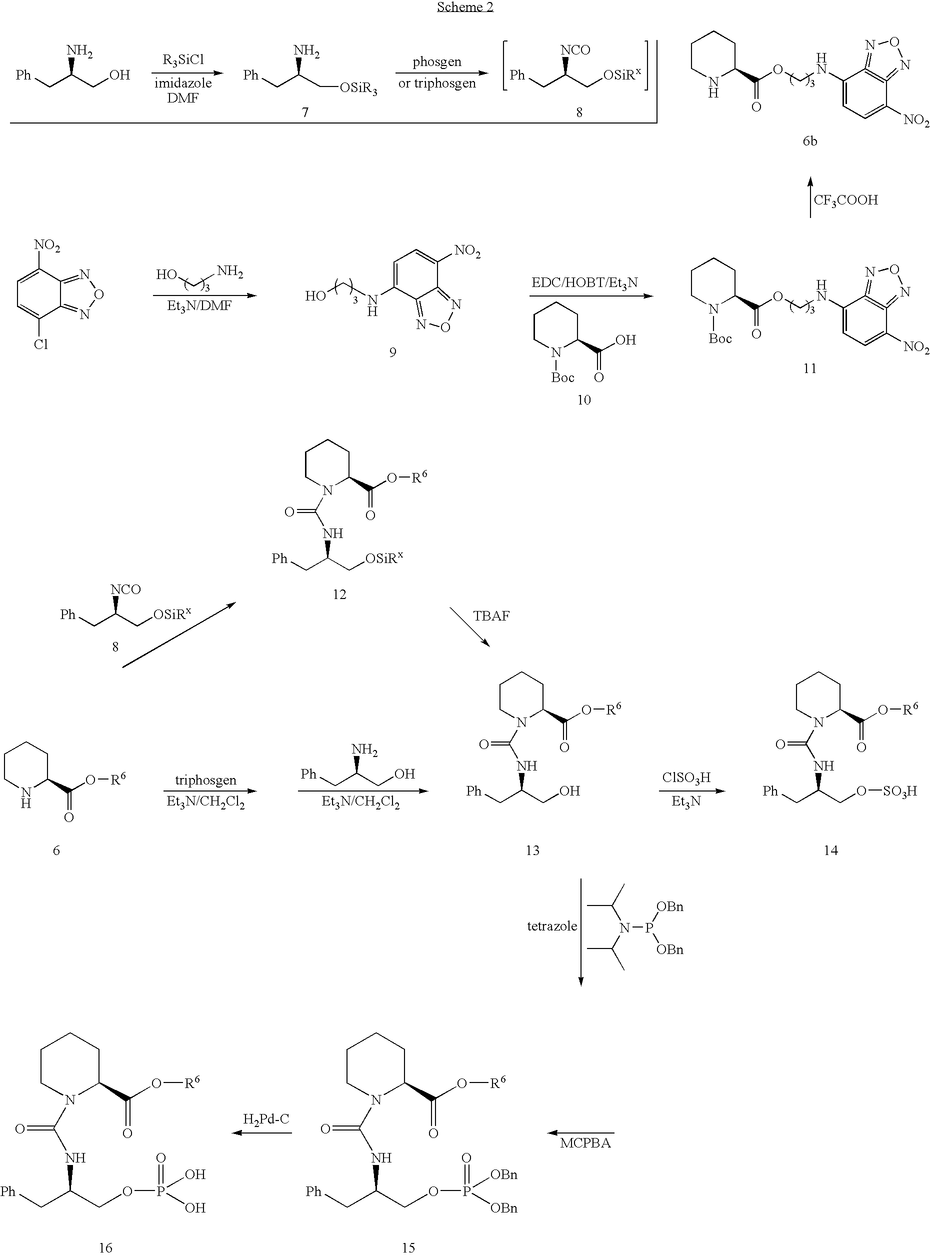 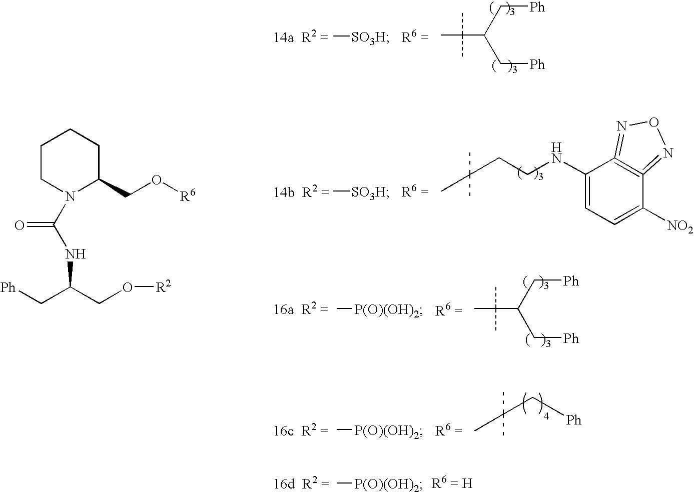 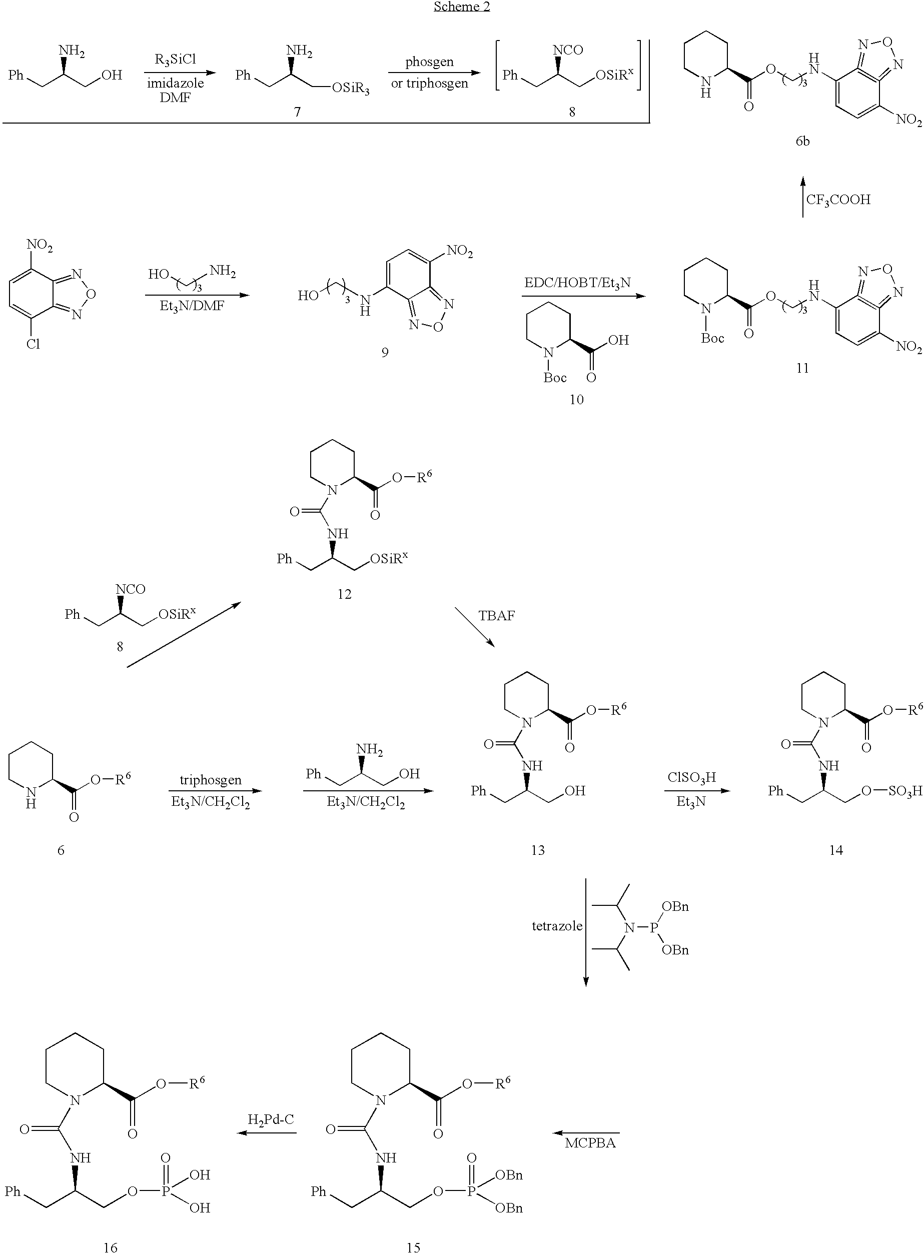 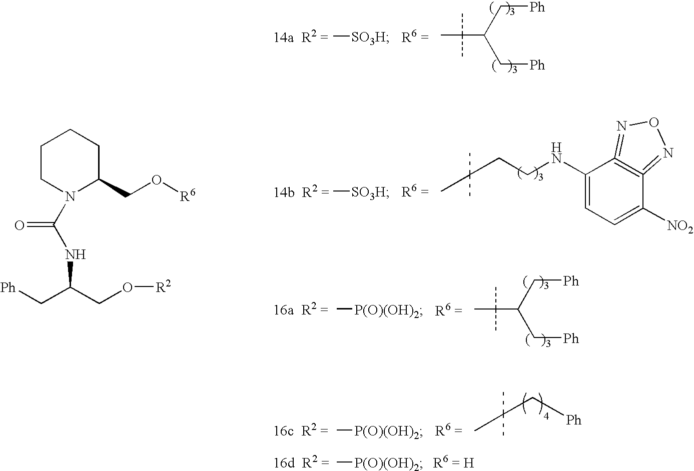
Alcohol 9:

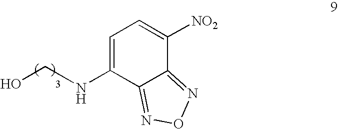

To a DMF solution (5 mL) of 3-amino-1-propanol (0.207 g, 0.21 mL, 2.75 mmol) was added triethylamine (0.42 mL, 3 mmol) and 4-chloro-7-nitrobenzofurazan (0.5 g, 2.5 mmol) at 25° C. After 20 h, the reaction mixture was poured into water (100 mL). The precipitate was collected by filtration. Recrystalization from warm methanol afforded 150 mg (25% yield) of the compound 9 as a yellow solid. ¹H NMR (CD₃OD): δ 9.49 (1H, s), 8.51 (1H, d, J=8.7 Hz), 6.41 (1H, d, J=8.7 Hz), 4.65 (1H, m), 3.62-3.44 (4H, m), 1.84 (2H, p, J=6.6 Hz); MS (positive ESP): 239 (M+H⁺); MS (negative ESP): 237 (M−H)⁻.
Ester 11:

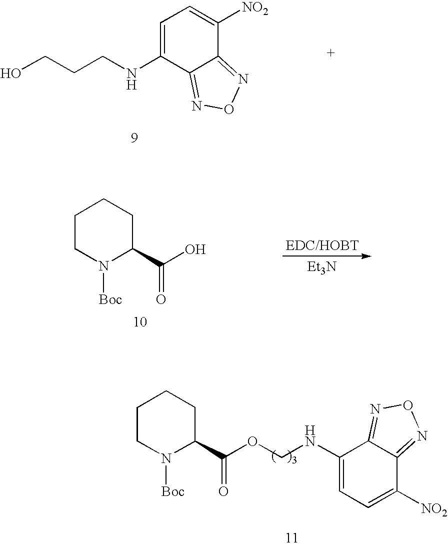

To a DMF solution (2 mL) of the alcohol 9 (0.08 g, 0.336 mmol) and N-Boc-pipecolinic acid 10 (0.115 g, 0.504 mmol, preparation described in International Publication No. WO 0140185) was added triethylamine (0.2 mL), EDC (1-(3-dimethylaminopropyl)-3-ethylcarbodiimide hydrochloride) (0.097 g, 0.504 mmol) and HOBT (1-hydroxybenzotriazole hydrate) (0.068 g, 0.0504 mmol) at 25° C. After 20 h, the reaction mixture was diluted with EtOAc (50 mL), washed with brine (3×50 mL), dried (Na₂SO₄) and concentrated. The residue was purified by column chromatography (20-25% EtOAc in hexanes) affording 150 mg (100% yield) of the compound 11. ¹H NMR (CDCl₃): (mixture of two rotamers) δ 8.48 (1H, d, J=8.7 Hz), 7.11-6.94 (1H, br s), 6.26 (1H, d, J=8.7 Hz), 4.85 (1H, br s), 4.36 (2H, m), 4.06-3.86 (2H, m), 3.67 (2H, m), 3.12-2.81 (1H, m), 2.26-2.12 (3H, m), 1.46 (9H, s).
Amine 6b

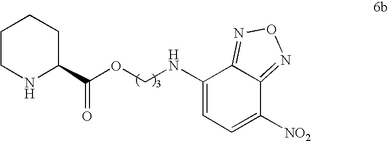

To a methylene chloride solution (5 mL) of the ester 11 (150 mg, 0.5 mmol) at −30° C. was added trifluoroacetic acid (1 mL). The solution was warmed to 25° C. over 3 h. All solvent was removed in vacuoto give 125 mg (100%) of the compound 6b. ¹H NMR (CDCl₃): δ8.45 (1H, d, J=8.4 Hz), 6.18 (1H, d, J=8.4 Hz), 4.35 (2H, t, J=6.3 Hz), 3.66 (2H, t, J=6 Hz), 3.43 (1H, dd, J=9.9, 3.3 Hz), 3.13 (1H, brd), 2.70 (1H, brt), 2.19 (2H, p, J=6.3 Hz).
Urea 12b

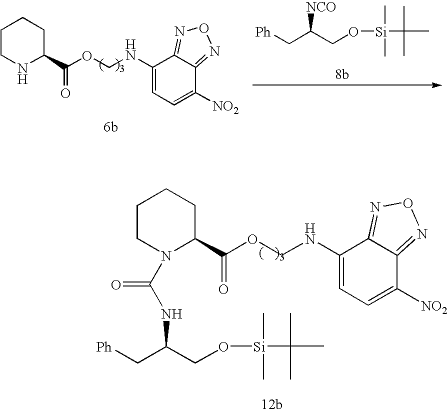

To a DMF solution (5 mL) of D-phenylalaninol (1 g, 6.6 mmol) was added imidazole (0.494 g, 7.27 mmol) and t-butyldimethylchlorosilane (1 g, 7.27 mmol). After 40 h, the mixture was diluted with ether (20 mL), washed with brine (3×50 mL) and dried (Na₂SO₄). All solvent was removed in vacuo to give the amine 8b (1.85 g) as a colorless oil. At −40° C., a portion of the amine 8b (0.265 g, 1 mmol) was added to a methylene chloride solution (5 mL) of phosgene (0.544 mL, 20% in toluene, 1.1 mmol) and triethylamine (0.5 mL). The solution was slowly warmed up to 25° C. over 30 min, and was cooled to 0° C. again. The pipecolate ester 6b (0.05 g, 0.143 mmol) was introduced at once. The mixture was stirred at 25° C. for 20 h, diluted with EtOAc (50 mL), washed with concentrated NaHCO₃ solution (1×50 mL), dried (Na₂SO₄) and concentrated. The residue was purified by column chromatography (50% EtOAc in hexanes) to afford 20 mg (22% yield) of the compound 12b. ¹H NMR (CDCl₃): δ 8.42 (1H, d, J=8.7 Hz), 7.30-7.07 (5H, m), 6.17 (1H, d, J=8.7 Hz), 5.11 (1H, d, J=8.7 Hz), 4.83 (1H, dd, J=6, 3.3 Hz), 4.37 (1H, m), 4.15 (1H, m), 4.02 (1H, m), 3.66-3.28 (4H, m), 3.19 (1H, td, J=11.7, 3.6 Hz), 2.81 (3H, m), 2.07 (3H, m), 0.88 (9H, s), 0.01 (3H, s), 0.0 (3H, s).
Alcohol 13b:

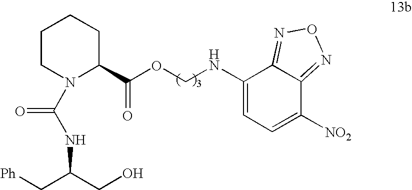

To a THF solution (4 mL) of the silyl ether (20 mg, 0.0313 mmol) was added tetrabutylammonium fluoride (1 mL, 1M in THF, 1 mmol). After 1 h at 25° C., the solution was concentrated. The resulting residue was purified by column chromatography (75% EtOAc in hexanes) to give 18 mg (100%) of the compound 13b. ¹H NMR (CD₃OD): δ 8.49 (1H, d, J=9 Hz), 7.3-7.1 (5H, m), 6.35 (1H, d, J=9 Hz), 4.38 (1H, m), 4.05 (1H, m), 3.92 (1H, dd, J=13.2, 4.8 Hz), 3.84-3.59 (6H, m), 3.13 (1H, m), 3.00 (1H, dd, J=13.8, 5.4 Hz), 2.78 (1H, m), 1.99 (3H, m).

### Example 14b

### 1-(2-Phenyl-1-sulfooxymethyl-ethylcarbamoyl)-piperidine-2-carboxylic acid 3-(7-nitro-benzo[1,2,5]oxadiazol-4-ylamino)-propyl ester

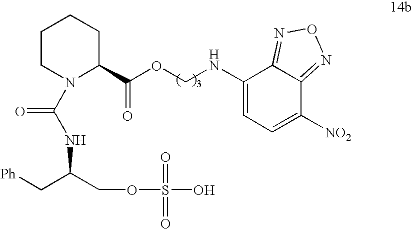

At −70° C., a methylene chloride solution (2 mL) of the alcohol 13b (10 mg, 0.019 mmol) was added triethylamine (0.05 mL) and chlorosulfonic acid (8 mg, 5 μl, 0.068 mmol). The cooling bath was then removed and the reaction mixture was allowed to warm to 25° C. over 3 h. All solvent was evaporated in vacuo. The residue was purified by column chromatography (10% MeOH in EtOAc) to give 5 mg (42% yield) of the title compound 14b. ¹H NMR (CD₃OD): δ 8.63 (1H, d, J=9 Hz), 7.37-7.18 (5H, m), 6.49 (1H, d, J=9 Hz), 4.71-4.53 (2H, m), 4.33-4.15 (3H, m), 3.98 (1H, dd, J=12.6, 4.5 Hz), 3.84-3.71 (4H, m), 3.36-3.11 (2H, m), 2.85 (1H, td, J=12.6, 4.5 Hz).

Alcohol 13a:

Method A:

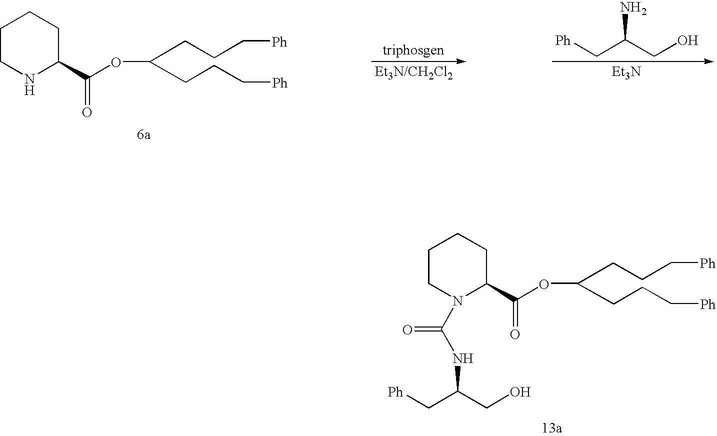

To a methylene chloride solution (4 mL) of the pipecolate ester 6a (0.2 g, 0.528 mmol) and triethylamine (1 mL) was added a methylene chloride solution (1 mL) of triphosgene (0.052 g, 0.176 mmol). After 10 min, the solution was heated at reflux for 1 h, and was then cooled to 25° C. A methylene chloride solution (1 mL) of D-phenylalaninol (0.0798 g, 0.528 mmol) was added. After 2 h, the reaction solution was diluted with Et₂O (50 mL), washed with brine (2×70 mL), dried (Na₂SO₄) and concentrated in vacuo. The residue was purified by flash column chromatography (20% EtOAc in hexanes) to afford 80 mg (27% yield) of the compound 13a. ¹H NMR (CDCl₃): δ 7.38-7.10 (15H, m), 4.96 (2H, m), 4.70 (1H, d, J=6.9 Hz), 4.01 (1H, m), 3.67 (1H, br d), 3.51 (1H, dd, J=10.8, 5.1 Hz), 3.25 (2H, brt), 3.04 (1H, td, J=12.3, 3.3 Hz), 2.84 (2H, m), 2.58 (4H, m), 2.18 (1H, brd, J=12.6 Hz); MS (ESP positive): 557 (M+H⁺); 555 (M−H)⁻.

Method B:

Silyl Ether 17:

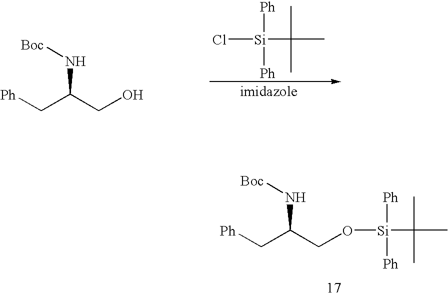

To a DMF solution (7 mL) of (R)-(+)-2-(t-Boc)-amino-3-phenyl-1-propanol (4.15 g, 16.5 mmol) was added imidazole (2.24 g, 33 mmol) and t-butylchlorodiphenylsilane (5.15 mL, 19.8 mmol). After 15 h at 25° C., the mixture was diluted with ether (50 mL), washed with sat'd NH₄Cl solution (3×50 mL), dried (Na₂SO₄) and concentrated in vacuo. The residue was purified by column chromatography (2-4% EtOAc in hexanes) to afford 9.19 g (100%) of the compound 17 as a white solid. MS (ESP): 512 (M+Na⁺).
Amine 7a

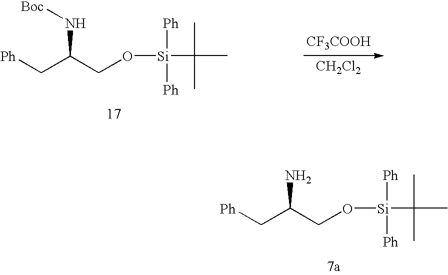

To a methylene chloride solution (60 mL) of the silyl ether 17 (9.19 g, 16.5 mmol) at 0° C. was added trifluoroacetic acid (20 mL). The reaction solution was stirred at 0° C. for 1 h and was then warmed to 25° C. over 30 min. The mixture was concentrated in vacuo and was redissolved in methylene chloride (50 mL). The resulting solution was washed with sat'd NaHCO₃ solution (2×50 mL), dried (Na₂SO₄) and concentrated. The residue was purified by column chromatography (97.5/2.5/0.25 CH₂Cl₂/MeOH/NH₄OH) to give 5.97 g (78% yield) of the compound 7a as a light yellow oil. ¹H NMR (CDCl₃): δ 7.56 (4H, br d), 7.36-7.23 (6H, m), 7.20-7.13 (2H, m), 7.12-7.02 (3H, m), 3.52 (1H, dd, J=9.9, 4.5 Hz), 3.43 (1H, dd, J=10.2, 6.3 Hz), 3.10-2.99 (1H, m), 2.71 (1H, dd, J=13.2, 4.8 Hz), 2.41 (1H, dd, J=13.5, 8.4 Hz), 0.97 (9H, s); MS (ESP): 390 (M+H⁺).
Urea 12a:

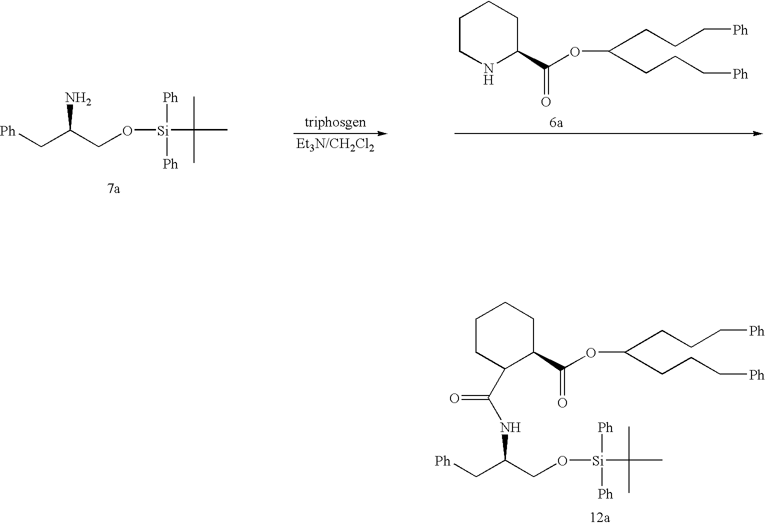

To a methylene chloride solution (20 mL) of the amine 7a (3.44 g, 7.04 mmol) and triethylamine (2 mL) was added a methylene chloride solution (1 mL) of triphosgene (0.613 g, 2.07 mmol). After 2 h, the solution was heated at reflux for 1.5 h, and was then cooled to 25° C. A methylene chloride solution (40 mL) of the amine 6a (2.67 g, 7.04 mmol) (preparation of 6a described in Guo et al. in International Publication No. WO 01/40183) was added After 15 h, the reaction solution was diluted with CH₂Cl₂ (100 mL), washed with brine (2×80 mL), dried (Na₂SO₄) and concentrated in vacuo. The residue was purified by flash column chromatography (5-20% EtOAc in hexanes) to afford 4.85 g (77% yield) of the compound 12a. ¹H NMR (CDCl₃): (mixture of two rotamers) δ 7.6-7.5 (4H, m), 7.39-7.23 (7H, m), 7.22-6.99 (14H, m), 5.00-4.80 (2H, m), 4.16 and 4.01 (1H, m), 3.51 (3H, m), 3.30 (1H, m), 3.07-2.74 (4H, m), 2.55-2.43 (2H, m), 1.04 and 1.03 (9H, s); MS (ESP): 817 (M+Na⁺).
Alcohol 13a:

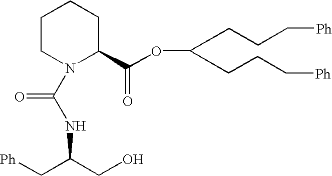

To a THF solution (30 mL) of the silyl ether 12a (4.83 g, 6.08 mmol) at 0° C. was added hydrogen fluoride-pyridine (8 mL). After 30 min at 0° C., the mixture was warmed to 25° C. over 1 h. All solvent was removed in vacuo. The residue was dissolved in methylene chloride (50 mL) and the solution was washed with ice-cold HCl solution (2×40 mL), dried (Na₂SO₄) and concentrated. Flash column chromatography purification provided 1.96 g (58% yield) of the compound 13a as a white solid.

### Example 14a

### 1-(2-Phenyl-1-sulfooxymethyl-ethylcarbamoyl)-piperidine-2S-carboxylic acid 4-phenyl-1-(3-phenyl-propyl)-butyl ester

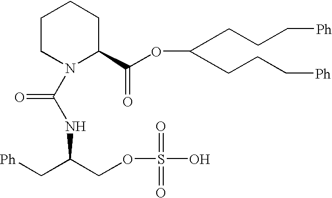

Prepared as described in the synthesis of 14b using the alcohol 13a (17 mg, 0.0306 mmol), chlorosulfonic acid (11 mg, 8 μl, 0.011 mmol) and triethylamine (0.05 mL). The reaction, mixture was diluted with EtOAc (20 mL) and washed with ice-cold 5% HCl solution (1×20 mL). Column chromatography (8% MeOH in EtOAc) afforded 12 mg (62% yield) of the title compound 14a. ¹H NMR (CD₃OD): δ 7.36-7.06 (15H, m), 6.55 (1H, d, J=7.8 Hz), 4.99 (1H, m), 4.11 (1H, m), 3.97 (2H, m), 3.74 (1H, m), 2.99 (1H, br t), 2.87 (2H, m), 2.60 (4H, m), 2.20 (1H, br d); MS (ESP negative): 635 (M−H)⁻; HRMS (FAB) calc for C₃₅H₄₃N₂O₇S(M−H) 635.2791; found 635.2815.
Phosphate Benzyl Ester 15a:

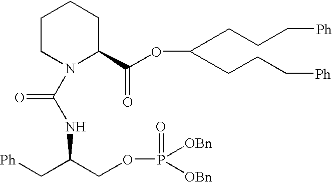

To an acetonitrile solution (10 mL) of the alcohol 13a (73 mg, 0.131 mmol) and 1H-tetrazole (15 mg, 0.21 mmol) was added dibenzyl N,N-diisopropylphosphoramidite (68 mg, 0.198 mmol) at 25° C. After 1 h, MCPBA (102 mg, 70% pure, 0.4 mmol) was added to the suspension. The solution was diluted with ether (50 mL), washed with concentrated NaHSO₃ solution (2×30 mL), dried over MgSO₄ and concentrated in vacuo. The residue was purified by column chromatography (20-25% EtOAc in hexanes) to give 70 mg of the compound 15a in 65% yield. ¹H NMR (CDCl₃): δ 7.40-7.07 (25H, m), 5.67 (1H, d, J=7.8 Hz), 5.10-4.89 (6H, m), 4.14 (1H, m), 3.90 (2H, m), 3.53 (1H, br d), 3.09 (1H, br t), 2.97 (1H, dd, J=13.2, 4.8 Hz), 2.69 (1H, dd, J=13.2, 9 Hz), 2.56 (4H, m), 2.18 (1H, br d).

### Example 16a

### 1-(2-Phenyl-1-phosphonooxymethyl-ethylcarbamoyl)-piperidine-2S-carboxylic acid 4-phenyl-1-(3-phenyl-propyl)-butyl ester

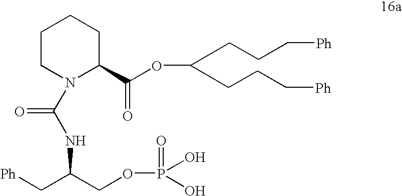

To a methanol solution of the phosphate benzyl ester 15a (50 mg, 0.061 mmol) was added palladium on carbon (10%, 6 mg). The suspension was kept under hydrogen (1 atm) for 5 h. After filtration, the filtrate was concentrated to dryness, affording 40 mg of the title compound 16a in 100% yield. ¹H NMR (CDCl₃): 7.36-7.07 (15H, m), 5.02 (1H, m), 4.86 (1H, br d), 4.12 (1H, m), 3.92 (2H, m), 3.70 (1H, br d), 3.09-2.88 (2H, m), 2.83 (1H, dd, J=13.8, 7.8 Hz), 2.60 (4H, m), 2.18 (1H, br d); HRMS (MALDI) calc for C₃₅H₄₄N₂07PNa₂ (M−H⁺+2Na⁺) 681.2681; found 681.2691.
Alcohol 13c:

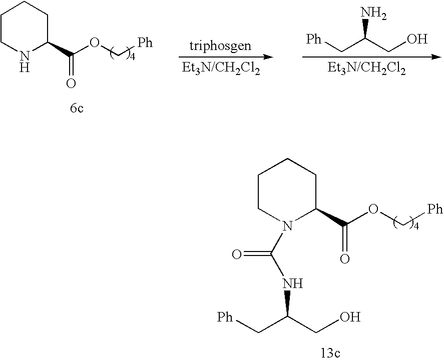

To a methylene chloride solution (10 mL) of the pipecolate ester 6c (0.5 g, 1.92 mmol, preparation described in International Publication No. WO 0140185) and triethylamine (2 mL) at −40° C. was added a toluene solution (1.04 mL) of phosgene (20%, 0.208 g, 0.176 mmol). The solution was warmed to 25° C. over 1 h and was then added D-phenylalaninol (0.29 g, 1.92 mmol). After 10 h, the reaction mixture was diluted with Et₂O (100 mL), washed with 5% ice-cold HCl solution (1×50 mL) and brine (1×50 mL), dried (Na₂SO₄) and concentrated in vacuo. The residue was purified by flash column chromatography (20% EtOAc in hexanes) to afford 100 mg (12% yield) of the compound 13c. ¹H NMR (CDCl₃): δ7.31-7.03 (10H, m), 4.85 (1H, brd), 4.1-3.9 (2H, m), 3.64 (1H, dd, J=10.8, 3.3 Hz), 3.50 (1H, dd, J=11.4, 5.7 Hz), 3.25 (1H, br d), 2.99 (1H, td, J=12.6, 3 Hz), 2.79 (2H, m), 2.56 (2H, m).
Phosphate Benzyl Ester 15c:

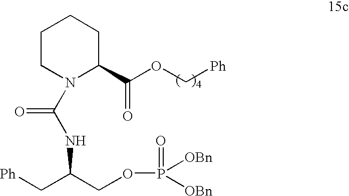

Prepared as described in the synthesis of 15a using the alcohol 13c (60 mg, 0.137 mmol), 1H-tetrazole (19.2 mg, 0.274 mmol), dibenzyl N,N-diisopropylphosphoramidite (0.069 mL, 71 mg, 0.205 mmol) and MCPBA (115 mg, 60% pure, 0.4 mmol). Preparative TLC purification (30% EtOAc in hexanes) provided 20 mg of the compound 15c in 21% yield. ¹H NMR (CDCl₃): δ 7.43-7.1 (20H, m), 5.64 (1H, d, J=7.5 Hz), 5.1-5.0 (4H, m), 4.97 (1H, br d), 4.21-3.82 (5H, m), 3.54 (1H, br d), 3.09 (1H, td, J=12.6, 3.3 Hz), 2.97 (1H, dd, J=13.5, 5.7 Hz), 2.70 (1H, dd, J=13.8, 9.6 Hz), 2.60 (2H, m), 2.19 (1H, d, J=13.5 Hz).

### Example 16c

### 1-(2-Phenyl-1-phosphonooxymethyl-ethylcarbamoyl)-piperidine-2S-carboxylic acid 4-phenyl-1-butyl ester

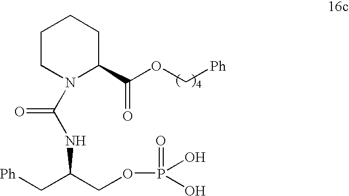

Prepared as described in the synthesis of 16a from phosphate benzyl ester 15c (20 mg, 0.457 mmol). Ethanol instead of methanol was used as a reaction solvent. The title compound 16c was obtained in 88% yield (13 mg). ¹H NMR (CD₃OD): δ 7.25-6.99 (10H, m), 4.01 (3H, m), 3.83 (2H, m), 3.62 (1H, br d), 2.94-2.66 (3H, m), 2.53 (2H, m), 2.08 (1H, d, J=12.9 Hz); HRMS (MALDI) calc for C₂₆H₃₅N₂O₇PNa (M+Na⁺) 541.2080; found 541.2106.
Alcohol 13d:

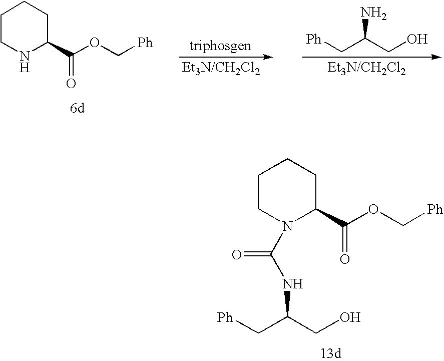

Prepared as described in the synthesis of 13c using pipecolate ester 6d (0.78 g, 3.56 mmol, preparation described in International Publication No. WO 0140185), triethylamine (2 mL), phosgene (20% in toluene, 2.4 mL, 4.45 mmol) and D-phenylalaninol (1.35 g, 8.9 mmol). Flash column chromatography purification (50% EtOAc in hexanes) to afford 60 mg (4.4% yield) of the compound 13d. ¹H NMR (CDCl₃): δ 7.31-7.16 (5H, m), 4.42 (1H, m), 4.06 (1H, dd, J=13.2, 4.8 Hz), 3.97-3.81 (2H, m), 3.58 (1H, dd, J=12, 4.2 Hz), 3.39 (1H, dd, J=9.6, 3.6 Hz), 3.14 (2H, m), 2.75 (1H, td, J=13.8, 3.9 Hz), 2.09 (1H, m), 1.91 (1H, m), 1.67 (1H, m); MS (ESP): 397 (M+H⁺).
Phosphate Benzyl Ester 15d:

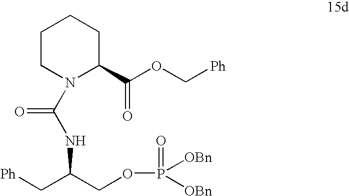

Prepared as described in synthesis of 15a using the alcohol 13d (89 mg, 0.225 mmol), 1H-tetrazole (32 mg, 0.45 mmol), dibenzyl N,N-diisopropylphosphoramidite (0.113 mL, 0.337 mmol) and MCPBA (136 mg, 60% pure, 0.45 mmol). Column chromatography purification (30% EtOAc in hexanes) provided 71 mg of the compound 15d in 48% yield. ¹H NMR (CDCl₃): δ 7.31-7.06 (20H, m), 5.62 (1H, d, J=7.8 Hz), 5.11-4.92 (2H, m), 4.13 (1H, m), 3.99-3.79 (2H, m), 3.49 (1H, br d), 3.01 (1H, td, J=12.3, 2.7 Hz), 2.89 (1H, dd, J=13.5, 5.4 Hz), 2.63 (1H, dd, J=13.8, 9.3 Hz), 2.15 (1H, br d, J=13.8 Hz); MS (ESP positive): 657 (M+H⁺), 679 (M+Na⁺).

### Example 16d

### 1-(2-Phenyl-1-phosphonooxymethyl-ethylcarbamoyl)-piperidine-2-carboxylic acid

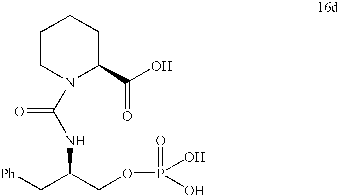

Prepared as described in the synthesis of 16a from phosphate benzyl ester 15d (48 mg, 0.073 mmol). Ethanol instead of methanol was used as a reaction solvent. The benzyl ester of the carboxylate was also cleaved to carboxylic acid during the hydrogenation. After HPLC purification, the title compound 16d was obtained in 4% yield (1 mg). ¹H NMR (CD₃OD): δ 7.46-7.11 (5H, m), 4.71-4.45 (2H, m), 4.19 (1H, m), 3.93 (1H, dd, J=13.2, 4.8 Hz), 3.69 (1H, dd, J=12, 4.2 Hz), 3.26-3.0 (2H, m), 2.79 (1H, td, J=13.2, 3.6 Hz), 2.00 (1H, m).

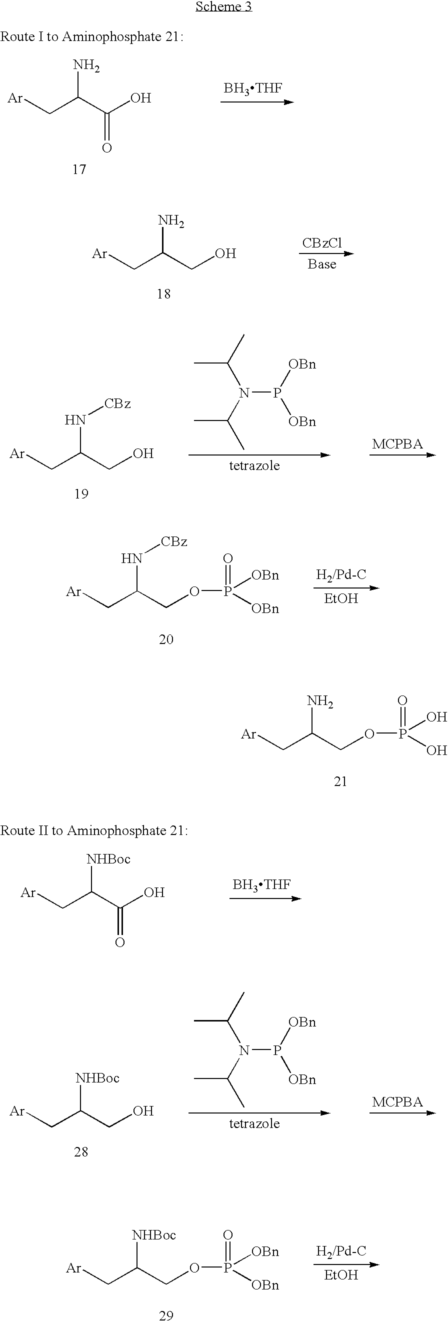

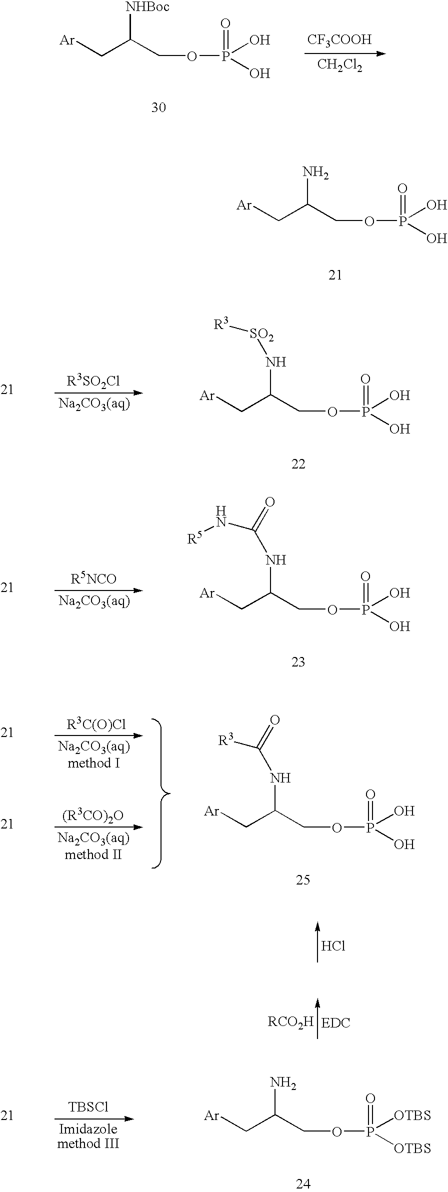

The following is a list of the compounds prepared using the synthetic pathways outlined in Scheme 3, and then the detailed experimental procedures.

Compounds Made Using Scheme 3

| 23a | R⁵ = | Ph | 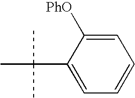 |
|---|---|---|---|
| 23b | R⁵ = | | 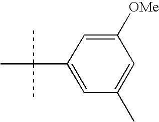 |
| 23c | R⁵ = | | 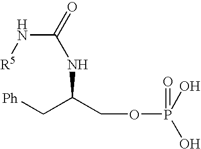 |
| 23d | R⁵ = | | 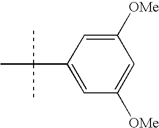 |
| 23e | | | 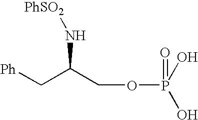 |
| 25-1 | R³ = | Ph | |
| 25-2 | R³ = | | 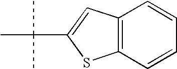 |
| 25-2′ | R³ = | | 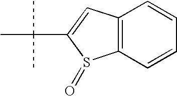 |
| 25-3 | R³ = | 2- naphthyl | 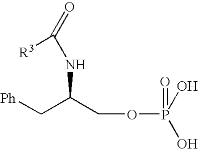 |
| 25-4 | R³ = | | 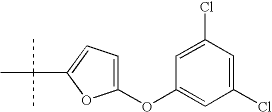 |
| 25-5 | R³ = | 3,4- dichloro- phenyl | |
| 25-6 | R³ = | | 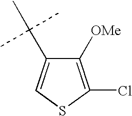 |
| 25-7 | R³ = | | 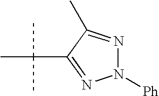 |
| 25-8 | R³ = | 3- | |
| | | chloro- | |
| | | phenyl | |
| 25-9 | R³ = | 3- | |
| | | pyridyl | |
| 25-10 | R³ = | 1- | |
| | | naphthyl | |
| 25-11 | R³ = | | 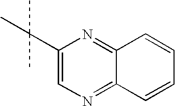 |
| 25-12 | R³ = | | 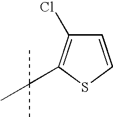 |
| 25-13 | R³ = | 2-hydro- xyphenyl | |
| 25-14 | R³ = | | 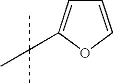 |
| 25-15 | R³ = | isopropyl | |
| 25-16 | R³ = | t-butyl | |
| 25-17 | R³ = | cyclohexyl | |
| 25-18 | R³ = | | 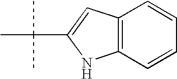 |
| 25-19 | R³ = | | 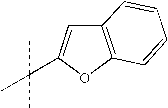 |
| 25-20 | R³ = | | 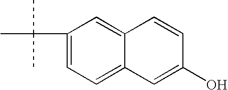 |
| 25-21 | R³ = | | 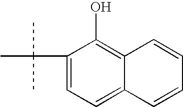 |
| 25-22 | R³ = | | 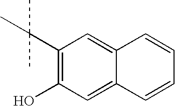 |
| 25-23 | R³ = | | 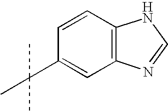 |
| 25-24 | R³ = | | 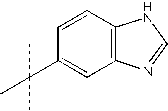 |
| 25-25 | R³ = | | 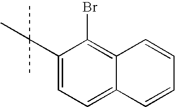 |
| 25-40 | R³ = | Me | 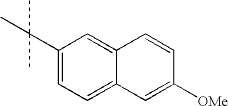 |

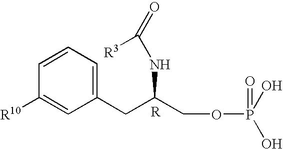

| | | | |
|---|---|---|---|
| 25-28 | R¹⁰ = | F, R³ = | 2-naphthyl |
| 25-29 | R¹⁰ = | F, R³ = | 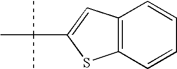 |
| 25-30 | R¹⁰ = | F, R³ = | 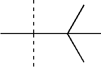 |
| 25-31 | R¹⁰ = | F, R³ = | 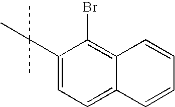 |
| 25-32 | R¹⁰ = | F, R³ = | 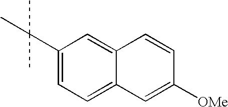 |
| 25-33 | R¹⁰ = | F, R³ = | 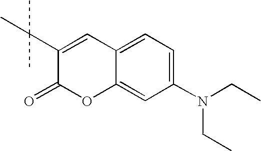 |
| 25-34 | R¹⁰ = | Me, R³ = | 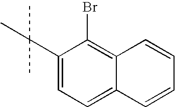 |
| 25-35 | R¹⁰ = | Me, R³ = | 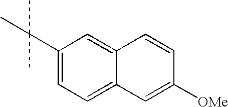 |
| 25-36 | R¹⁰ = | Me, R³ = | 2-naphthyl |
| 25-37 | R¹⁰ = | Me, R³ = | 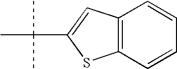 |

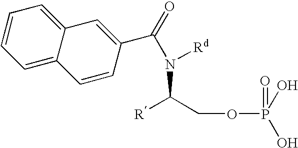

| | | | |
|---|---|---|---|
| 33 | R^{d} = | H, R′ = | 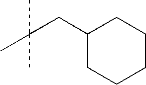 |
| 37 | R^{d} = | H, R′ = | 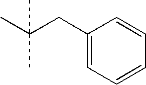 |
| 45 | R^{d} = | R′ = | H |
| 58 | R^{d} = | Me, R′ = | Benzyl |

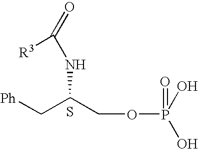

| | | | |
|---|---|---|---|
| 25-26 | R³ = | | 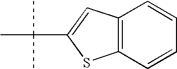 |
| 25-27 | R³ = | 2-naphthyl | |

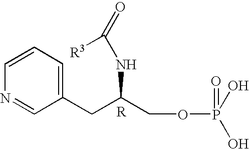

| | | |
|---|---|---|
| 25-38 | R³ = | 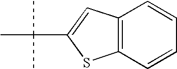 |
| 25-39 | R³ = | 2-naphthyl |

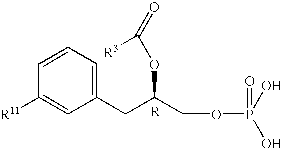

| 49 | R¹¹ = | H, R³ = | 2-naphthyl |
|---|---|---|---|
| 54 | R¹¹ = | F, R³ = | 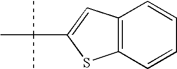 |
| 42 | | | 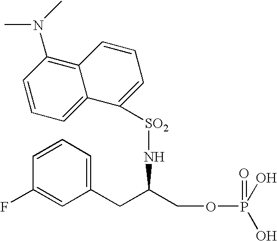 |

Alcohol 19a

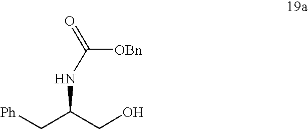

To a methylene chloride solution (80 mL) of D-phenylalaninol 18a (1.15 g, 7.61 mmol) was added triethylamine (1.59 mL, 11.4 mmol) and benzyl chloroformate (1.19 mL, 8.37 mmol). The mixture was stirred for 3 h and then concentrated. The residue was dissolved in methylene chloride (50 mL) and washed with brine (1×50 mL). The solution was dried (Na₂SO₄) and concentrated. After column chromatography purification (10 to 30% EtOAc in hexanes), the compound 19a was obtained in 73% yield (1.59 g). ¹H NMR (CDCl₃): δ 7.46-7.15 (10H, m), 5.11 (2H, s), 4.96 (1H, m), 3.98 (1H, m), 3.72 (1H, m), 3.63 (1H, m), 2.89 (1H, d, J=7.2 Hz); MS (ESP): 286 (M+H⁺); 284 (M−H)⁻.
Alcohol 19b:

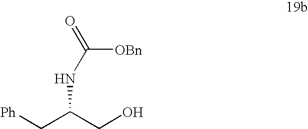

Prepared as described in the synthesis of the alcohol 19a using L-phenylalaninol 18b (2.59 g, 17.1 mmol), triethylamine (2.6 g, 3.58 mL, 25.7 mmol) and benzyl chloroformate (2.69 mL, 18.8 mmol). After column chromatography purification (20 to 40% EtOAc in hexanes), the compound 19b was obtained in 55% yield (2.61 g). ¹H NMR (CDCl₃): δ 7.43-7.13 (10H, m), 5.09 (2H, s), 4.94 (1H, m), 3.95 (1H, m), 3.69 (1H, m), 3.58 (1H, m), 2.87 (1H, d, J=7.2 Hz); MS (ESP): 286 (M+H⁺); 284 (M−H)⁻.
Phosphate Benzyl Ester 20a:

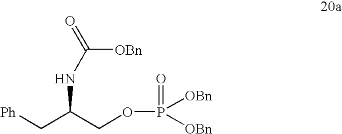

To an acetonitrile solution (40 mL) of the alcohol 19a (1.58 g, 5.54 mmol) and 1H-tetrazole (1.05 g, 15 mmol) was added dibenzyl N,N-diisopropylphosphoramidite (3.72 mL, 11.1 mmol) at 25° C. After 3 h, MCPBA (4.19 g, 70% pure, 13.85 mmol) was added to the suspension. The solution was diluted with EtOAc (100 mL), washed with concentrated NaHSO₃ solution (2×80 mL), dried over MgSO₄ and concentrated in vacuo. The residue was purified by column chromatography (10-30% EtOAc in hexanes) to give 2.88 g of the compound 20a in 95% yield. ¹H NMR (CDCl₃): δ 7.47-7.05 (20H, m), 5.19-4.96 (7H, m), 4.09-3.83 (3H, m), 2.93-2.67 (2H, m); MS (positive ESP): 568 (M+Na⁺); MS (negative ESP): 580 (M+Cl)⁻.
Phosphate Benzyl Ester 20b:

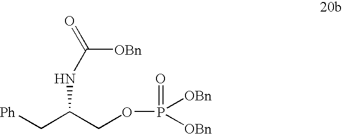

Prepared as described in the synthesis of compound 20a using the alcohol 19a (2.61 g, 9.16 mmol), 1H-tetrazole (1.73 g, 24.7 mmol), dibenzyl N,N-diisopropylphosphoramidite (6.15 mL, 18.3 mmol) and MCPBA (6.26 g, 77% pure, 27.5 mmol). Purification by column chromatography (15-30% EtOAc in hexanes) gave 4.1 g of the compound 20b in 82% yield. ¹H NMR (CDCl₃): δ 7.42-7.1 (20H, m), 5.16-5.0 (7H, m), 4.09-3.84 (3H, m), 2.9-2.69 (2H, m); MS (ESP): 546 (M+H⁺); 580 (M+Cl)⁻.
Aminophosphate 21a:

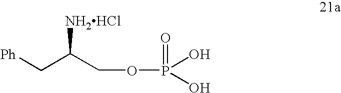

To an ethanol solution of the phosphate benzyl ester 20a (2.88 g, 5.28 mmol) was added palladium on carbon (10%, 300 mg). The suspension was kept under hydrogen atmosphere (1 atm) for 4 h, and was then filtered through a pad of celite. The collected solid was washed with methylene chloride. The mixture of the solid and celite was suspended in 5% HCl solution and stirred for 20 min. After filtration, the filtrate was concentrated to dryness, affording 1.2 g of the compound 21a in 86% yield. ¹H NMR (CD₃OD): δ 7.49-7.25 (5H, m), 4.22-4.08 (1H, m), 4.0 (1H, m), 3.72 (1H, m), 3.03 (2H, d, J=7.5 Hz); LCMS: 232 (M+H⁺); 230 (M−H)⁻; HRMS (MALDI) calc for C₉H₁₅NO₄P (M+H⁺) 232.0733; found 232.0736.
Aminophosphate 21b:

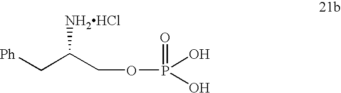

Prepared as described in the synthesis of 21a using phosphate benzyl ester 20b (4.1 g, 7.5 mmol) and palladium on carbon (10%, 410 mg). After filtration and evaporation, the compound 21b was obtained in quantitative yield (2.29 g). ¹H NMR (CD₃OD): δ 7.48-7.24 (5H, m), 4.14 (1H, m), 3.98 (1H, m), 3.69 (1H, m), 3.00 (2H, d, J=7.4 Hz); LCMS: 232 (M+H⁺).

### Example 23a

### Phosphoric acid mono-[3-phenyl-2-(3-phenyl-ureido)-propyl]ester

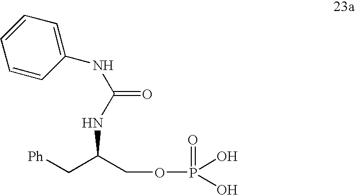

To a sodium carbonate solution (1 M, 1 mL) was added the aminophosphate 21a (53 mg, 0.198 mmol) and phenylisocyanate (0.023 mL, 0.208 mmol). After 15 h, it was acidified to pH˜1 by addition of concentrated HCl solution at 0° C. Preparative HPLC purification afforded 27 mg (42% yield) of the title compound 23a. ¹H NMR (CD₃OD): δ 7.36-7.17 (9H, m), 6.97 (1H, t, J=7.8 Hz), 4.19 (1H, m), 3.98 (2H, m), 2.98 (1H, dd, J=13.9, 7.1 Hz), 2.88 (1H, dd, J=13.8, 7.7 Hz); HRMS (MALDI) calc for C₁₆H₁₉N₂O₅PNa (M+Na⁺) 373.0924; found 373.0934.

### Example 23b

### Phosphoric acid mono-{2-[3-(2-phenoxy-phenyl)-ureido]-3-phenyl-propyl}ester

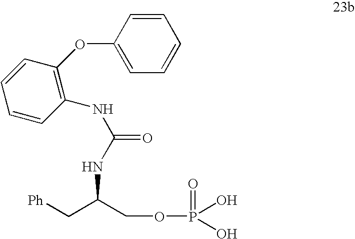

Prepared as described in the synthesis of 23a using 21a (82 mg, 0.307 mmol), 1-isocyanato-2-phenoxybenzene (67.8 mg, 0.058 mL, 0.322 mmol) and 1 M sodium carbonate solution (1 mL). Preparative HPLC purification gave 36 mg (27% yield) of the title compound 23b. ¹H NMR (CD₃OD): δ 8.05 (1H, d, J=8.4 Hz), 7.36 (2H, t, J=8.1 Hz), 7.30-7.02 (7H, m), 7.02-6.91 (3H, m), 6.83 (1H, d, J=8.1 Hz), 4.18 (1H, m), 3.95 (2H, m), 2.95 (1H, dd, J=13.8, 6.9 Hz), 2.81 (1H, dd, J=13.8, 7.8 Hz); MS (ESP): 443 (M+H⁺), 465 (M+Na⁺); 441 (M−H)⁻.

### Example 23c

### Phosphoric acid mono-{2-[3-(3-methoxy-5-methyl-phenyl)-ureido]-3-phenyl-propyl}ester

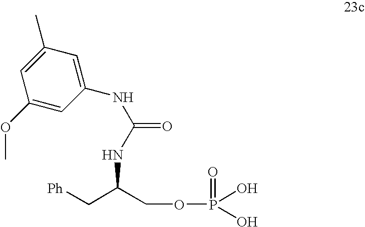

Prepared as described in the synthesis of 23a using 21a (62 mg, 0.232 mmol), 2-methoxy-5-methylphenylisocyanate (40 mg, 0.243 mmol) and 1 M sodium carbonate solution (1 mL). Preparative HPLC purification gave 50 mg (55% yield) of the title compound 23c. ¹H NMR (CD₃OD): δ 7.76 (1H, d, J=1.8 Hz), 7.33-7.27 (4H, m), 7.22 (1H, m), 6.81 (1H, d, J=8.1 Hz), 6.74 (1H, br d), 4.19 (1H, m), 3.97 (2H, m), 2.98 (1H, dd, J=13.9, 7.0 Hz), 2.83 (1H, dd, J=14, 8.1 Hz); HRMS (MALDI) calc for C₁₈H₂₄N₂O₆P (M+H⁺) 395.1372; found 395.1383.

### Example 23d

### Phosphoric acid mono-{2-[3-(3,5dimethoxy-phenyl)-ureido]-3-phenyl-propyl}ester

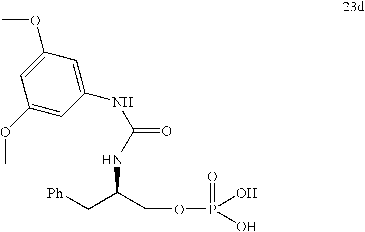

Prepared as described in the synthesis of 23a using 21a (71 mg, 0.265 mmol), 2,4-dimethoxy-phenylisocyanate (50 mg, 0.279 mmol) and 1 M sodium carbonate solution (1 mL). Preparative HPLC purification gave 29 mg (27% yield) of the title compound 23d. ¹H NMR (CD₃OD): δ 7.76 (1H, d, J=9 Hz), 7.46-7.29 (5H, m), 6.66 (1H, d, J=2.6 Hz), 6.56 (1H, dd, J=9, 2.8 Hz), 4.30 (1H, m), 4.08 (2H, m), 3.95 (3H, s), 3.88 (3H, s), 3.09 (1H, dd, J=113.8, 6.6 Hz), 2.96 (1H, dd, J=13.8, 7.7 Hz); MS (ESP) 411 (M+H⁺), 433 (M+Na⁺); 409 (M−H)⁻.

### Example 23e

### Phosphoric acid mono-(2-benzenesulfonylamino-3-phenyl-propyl)ester

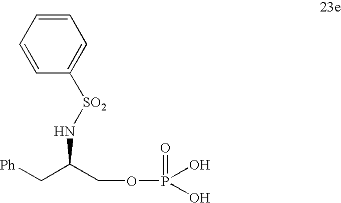

To a sodium carbonate solution (1 M, 1 mL) was added the aminophosphate 21a (66 mg, 0.246 mmol) and phenylsulfonyl chloride (0.047 mL, 0.369 mmol). After 15 h, it was acidified to pH˜1 by addition of concentrated HCl solution at 0° C. Preparative HPLC purification afforded 35 mg (38% yield) of the title compound 23e. ¹H NMR (CD₃OD): δ 7.67 (2H, d, J=7.5 Hz), 7.54 (1H, t, J=7.2 Hz), 7.42 (1H, t, J=7.8 Hz), 7.21-7.11 (3H, m), 7.09-7.03 (2H, m), 3.95 (1H, m), 3.84 (1H, m), 3.61 (1H, m), 2.94 (1H, dd, J=13.8, 6.6 Hz), 2.59 (1H, dd, J=13.5, 7.8 Hz); LCMS: 372 (M+H⁺), 394 (M+Na⁺); 370 (M−H)⁻; HRMS (MALDI) calc for C₁₅H₁₈NO₆PSNa (M+Na⁺) 394.0485; found 394.0487.

### Example 25-1

### Phosphoric acid mono-{3-phenyl-2-[(1-phenyl-methanoyl)-amino]-propyl}ester

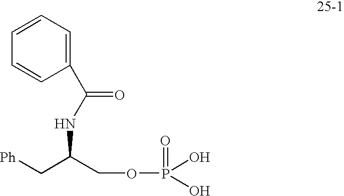

To a sodium carbonate solution (1 M, 1 mL) was added the aminophosphate 21a (70 mg, 0.262 mmol) and benzyol chloride (0.028 mL, 0.238 mmol). After 15 h, it was acidified to pH˜1 by addition of concentrated HCl solution at 0° C. Preparative HPLC purification afforded 20 mg (23% yield) of the title compound 25-1. ¹H NMR (CD₃OD): δ 8.04 (1H, br d), 7.76 (2H, br d), 7.76 (2H, br d), 7.64-7.16 (9H, m), 4.52 (1H, m), 4.09 (2H, m), 3.08 (1H, dd, J=13.6, 6.8 Hz), 2.96 (1H, dd, J=13.5, 8.1 Hz); MS (ESP): 336 (M+H⁺); 334 (M−H)⁻.

### Example 25-2

### Phosphoric acid mono-{(R)-2-[(1-benzo[b]thiophen-2-yl-methanoyl)-amino]-3-phenyl-propyl}ester

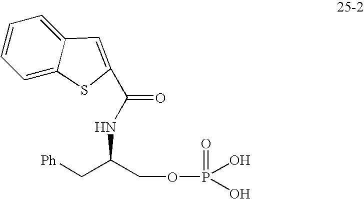

Prepared as described in the synthesis of 25-1 using 21a (48 mg, 0.179 mmol), benzothiophene-2-carbonyl chloride (35 mg, 0.179 mmol) and 1 M sodium carbonate solution (1 mL). Preparative HPLC purification gave 34 mg (48% yield) of the title compound 25-2. ¹H NMR (CD₃OD): δ 7.96 (1H, s), 7.90 (2H, m), 7.43 (2H, m), 7.37-7.17 (5H, m), 4.50 (1H, m), 4.10 (2H, m), 3.09 (1H, dd, J=13.9, 6.6 Hz), 3.00 (1H, dd, J=13.9, 7.8 Hz); HRMS (MALDI) calc for C₁₈H₁₈NO₅PSNa (M+Na⁺) 414.0540; found 414.0536.

### Example 25-2′

### Phosphoric acid mono-(2-{[1-(1-oxo-benzo[b]thiophen-2-yl)-methanoyl]-amino}-3-phenyl-propyl)ester

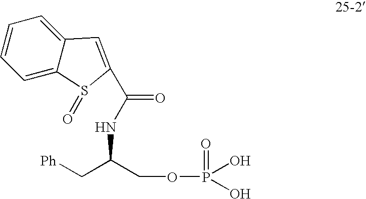

To a trifluoroacetic acid solution (1 mL) of 25-2, 9 mg, 0.023 mmol) at 0° C. was added 30% hydrogen peroxide (0.0244 mL). The solution was concentrated in vacuo. The residue was purified by preparative HPLC to give 1 mg (10% yield) of the title compound 25-2′. ¹H NMR (CD₃OD): δ 8.01-7.93 (2H, m), 7.78-7.6 (3H, m), 7.36-7.26 (4H, m), 7.25-7.17 (1H, m), 4.44 (1H, m), 4.04 (2H, m), 3.02 (2H, m); MS (ESP): 408 (M+H⁺), 430 (M+Na⁺).

### Example 25-3

### Phosphoric acid mono-{(R)-2-[(1-naphthalen-2-yl-methanoyl)-amino]-3-phenyl-propyl}ester

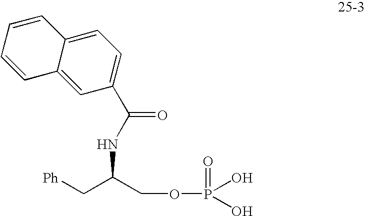

Prepared as described in the synthesis of 25-1 using 21a (50 mg, 0.187 mmol), 2-naphthoyl chloride (36 mg, 0.187 mmol) and 1 M sodium carbonate solution (1 mL). Preparative HPLC purification gave 29 mg (40% yield) of the title compound 25-3. ¹H NMR (CD₃OD): δ 8.32 (1H, s), 8.1-7.88 (3H, m), 7.83 (1H, dd, J=8.4, 1.8 Hz), 7.64-7.53 (2H, m), 7.39-7.18 (5H, m), 4.57 (1H, m), 4.12 (2H, m), 3.12 (1H, dd, J=13.7, 6.8 Hz), 3.02 (1H, dd, J=13.7, 8.1 Hz); LCMS (ESP):. 386 (M+H⁺), 408 (M+Na⁺); 384 (M−H)⁻; HRMS (MALDI) calc for C₂₀H₂₀NO₅PNa (M+Na⁺) 408.0971; found 408.0986.

### Example 25-4

### Phosphoric acid mono-[2-({1-[5-(3,5-dichloro-phenoxy)-furan-2-yl]-methanoyl}-amino)-3-phenyl-propyl]ester

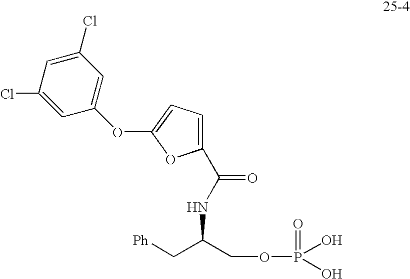

Prepared as described in the synthesis of 25-1 using 21a (76 mg, 0.284 mmol), 5-(3,5-dichlorophenoxy)-2-furoyl chloride (83 mg, 0.284 mmol) and 1 M sodium carbonate solution (1 mL). Preparative HPLC purification gave 46 mg (33% yield) of the title compound 254. ¹H NMR (CD₃OD): δ 7.21 (1H, t, J=1.7 Hz), 7.17-7.03 (5H, m), 7.00 (2H, d, J=1.8 Hz), 6.99 (1H, d, J=3.6 Hz), 5.79 (1H, d, J=3.9 Hz), 4.35 (1H, m), 3.92 (2H, m), 2.91 (1H, dd, J=13.6, 6 Hz), 2.78 (1H, dd, J=13.8, 8.7 Hz); MS (ESP): 508 (M+Na⁺); 484 (M−H)⁻.

### Example 25-5

### Phosphoric acid mono-(2-{[1-(3,4-dichloro-phenyl)-methanoyl]amino}-3-phenyl-propyl)ester

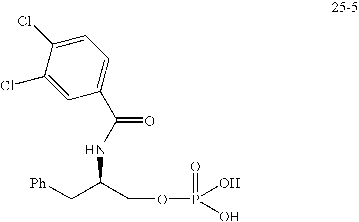

Prepared as described in the synthesis of 25-1 using 21a (56 mg, 0.209 mmol), 3,4-dichlorobenzoyl chloride (44 mg, 0.209 mmol) and 1 M sodium carbonate solution (1 mL). Preparative HPLC purification gave 20 mg (24% yield) of the title compound 25-5. ¹H NMR (CD₃OD): δ 7.91 (1H, d, J=2.1 Hz), 7.68 (1H, dd, J=8.4, 1.8 Hz), 7.60 (1H, d, J=8.4 Hz), 7.35-7.16 (5H, m), 4.50 1H, m), 4.08 (2H, m), 3.07 (1H, dd, J=13.9, 6.8 Hz), 2.94 (1H, dd, J=13.9, 8.4 Hz); LCMS (ESP): 404 (M+H⁺), 426 (M+Na⁺); 402 (M−H)⁻.

### Example 25-6

### Phosphoric acid mono-(2{[1-(5-chloro-4-methoxy-thiophen-3-yl)-methanoyl]-amino}-3-phenyl-propyl)ester

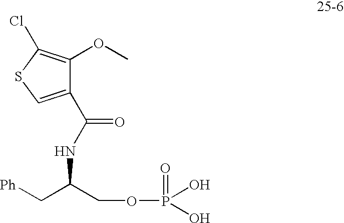

Prepared as described in the synthesis of 25-1 using 21a (63 mg, 0.236 mmol), 2-chloro-3-methoxythiophene-4-carbonyl chloride (50 mg, 0.236 mmol) and 1 M sodium carbonate solution (1 mL). Preparative HPLC purification gave 37 mg (38% yield) of the title compound 25-6. ¹H NMR (CD₃OD): δ 7.86 (1H, s), 7.38-7.19 (5H, m), 4.51 (1H, m), 4.09 (2H, m), 3.93 (2H, m), 3.04 (2H, m); LCMS (ESP): 406 (M+H⁺), 428 (M+Na⁺); 404 (M−H)⁻.

### Example 25-7

### Phosphoric acid mono-(2-{[1-(5-methyl-2-phenyl-2H-[1,2,3]triazol-4-yl)-methanoyl]-amino}-3-phenyl-propyl)ester

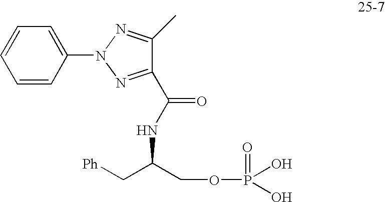

Prepared as described in the synthesis of 25-1 using 21a (46 mg, 0.172 mmol), 4-methyl-2-phenyl-1,2,3-triazole-5-carbonyl chloride (38 mg, 0.172 mmol) and 1 M sodium carbonate solution (1 mL). Preparative HPLC purification gave 36 mg (50% yield) of the title compound 25-7. ¹H NMR (CD₃OD): δ 7.98 (1H, d, J=8.1 Hz), 7.43 (2H, br t), 7.31 (1H, br t), 7.25-7.04 (5H, m), 4.40 (1H, m), 3.98 (2H, m), 2.96 (1H, dd, J=13.7, 6.8 Hz), 2.87 (1H, dd, J=13.9, 7.9 Hz); LCMS (ESP): 417 (M+H⁺), 439 (M+Na⁺); 415 (M−H)⁻.

### Example 25-8

### Phosphoric acid mono-(2-{[1-(3-chloro-phenyl)-methanoyl]-amino}-3-phenyl-propyl) ester

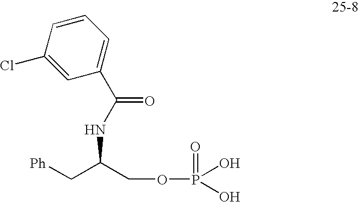

Prepared as described in the synthesis of 25-1 using 21a (45 mg, 0.168 mmol), 3-chlorobenzoyl chloride (29 mg, 0.168 mmol) and 1 M sodium carbonate solution (1 mL). Preparative HPLC purification gave 5 mg (8% yield) of the title compound 25-8. ¹H NMR (CD₃OD): δ 7.64 (1H, s), 7.57 (1H, d, J=7.5 Hz), 7.39 (1H, d, J=9 Hz), 7.30 (1H, t, J=7.8 Hz), 7.23-7.01 (5H, m), 4.33 (1H, m), 3.91 (2H, m), 2.94 (1H, dd, J=13.5, 6.9 Hz), 2.83 (1H, dd, J=13.8, 8.1 Hz); LCMS (ESP): 392 (M+Na⁺); 368 (M−H)⁻.

### Example 25-9

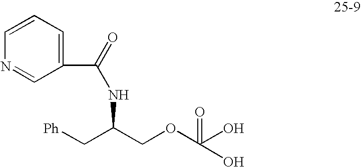

Prepared as described in the synthesis of 25-1 using 21a (46 mg, 0.172 mmol), nicotinoyl chloride hydrochloride (31 mg, 0.172 mmol) and 1 M sodium carbonate solution (1 mL). Preparative HPLC purification gave 3 mg (2% yield) of the title compound 25-9. ¹H NMR (CD₃OD): δ 8.89 (1H, s), 8.68 (1H, d, J=5.1 Hz), 8.36 (1H, dt, J=7.5, 1.8 Hz), 7.68 (1H, dd, J=7.8, 5.4 Hz), 7.22-7.05 (5H, m), 4.42 (1H, m), 3.98 (2H, m), 2.96 (1H, dd, J=13.5, 6.6 Hz), 2.85 (1H, dd, J=13.5, 8.1 Hz); LCMS (ESP): 337 (M+H⁺), 359 (M+Na⁺); 335 (M−H)⁻.

### Example 25-10

### Phosphoric acid mono-{2-[(1-naphthalen-1-yl-methanoyl)-amino]-3-phenyl-propyl}ester

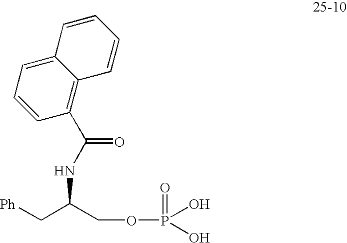

Prepared as described in the synthesis of 25-1 using 21a (62 mg, 0.232 mmol), 1-naphthoyl chloride (0.035 mL, 0.232 mmol) and 1 M sodium carbonate solution (1 mL). Preparative HPLC purification gave 8 mg (9% yield) of the title compound 25-10. ¹H NMR (CD₃OD): δ 7.93 (1H, m), 7.88 (1H, d, J=7.8 Hz), 7.77 (1H, d, J=8.4 Hz), 7.55-7.24 (9H, m), 4.70 (1H, m), 4.16 (2H, m), 3.14 (1H, dd, J=14.1, 5.8 Hz), 2.91 (1H, dd, J=13.9, 9.4 Hz); LCMS (ESP): 408 (M+Na⁺), 430 (M−H+2Na⁺); 384 (M−H)⁻.

### Example 25-11

### Phosphoric acid mono-{3-phenyl-2-[(1-quinoxalin-2-yl-methanoyl)-amino]-propyl}ester

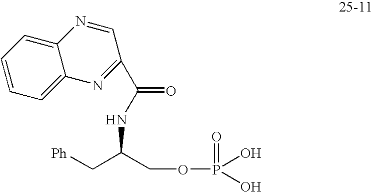

Prepared as described in the synthesis of 25-1 using 21a (68 mg, 0.254 mmol), quinoxaloyl chloride (50 mg, 0.254 mmol) and 1 M sodium carbonate solution (1 mL). Preparative HPLC purification gave 17 mg (17% yield) of the title compound 25-11. ¹H NMR (CD₃OD): δ 9.44 (1H, s), 8.24 (1H, m), 8.17 (1H, m), 8.01-7.90 (2H, m), 7.4-7.14 (5H, m), 4.62 (1H, m), 4.17 (2H, m), 3.10 (2H, m); LCMS (ESP): 388 (M+H⁺), 410 (M+Na⁺); 386 (M−H)⁻.

### Example 25-12

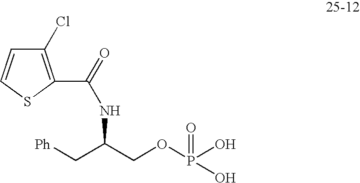

Prepared as described in the synthesis of 25-1 using 21a (63 mg, 0.236 mmol), 3-chloro-thiophene-2-carbonyl chloride (43 mg, 0.236 mmol) and 1 M sodium carbonate solution (1 mL). Preparative HPLC purification gave 26 mg (30% yield) of the title compound 25-12. ¹H NMR (CD₃OD): δ 7.67 (1H, d, J=5.1 Hz), 7.37-7.18 (5H, m), 7.05 (1H, d, J=5.4 Hz), 4.50 (1H, m), 4.09 (2H, m), 3.08 (1H, dd, J=13.9, 6.9 Hz), 2.99 (1H, dd, J=13.9, 8.5 Hz); HRMS (MALDI) calc for C₁₄H₁₆NO₅PSCI (M+H⁺) 376.0175; found 376.0158.

### Example 25-13

### Phosphoric acid mono-(2-{[1-(2-hydroxy-phenyl)-methanoyl]-amino}-3-phenyl-propyl)ester

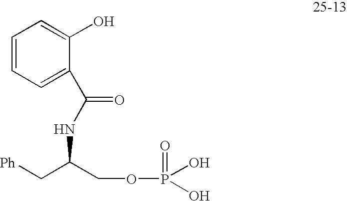

Prepared as described in the synthesis of 25-1 using 21a (187 mg, 0.699 mmol), acetylsalicyloyl chloride (139 mg, 0.699 mmol) and 1 M sodium carbonate solution (2 mL). Preparative HPLC purification gave 25 mg (10% yield) of the title compound 25-13. ¹H NMR (CD₃OD): δ 7.79 (1H, dd, J=8.1, 1.8 Hz), 7.41-7.2 (7H, m), 6.89 (2H, m), 4.53 (1H, m), 4.08 (2H, m), 3.07 (1H, dd, J=13.6, 6.8 Hz), 2.99 (1H, dd, J=13.6, 6.7 Hz); HRMS (MALDI) calc for C₁₆H₁₉NO₆P (M+H⁺) 352.0950; found 352.0960.

### Example 25-14

### Phosphoric acid mono-{2-[(1-furan-2-yl-methanoyl)-amino]-3-phenyl-propyl}ester

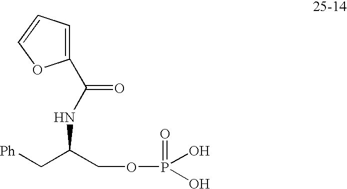

Prepared as described in the synthesis of 25-1 using 21a (85 mg, 0.318 mmol), 2-furoyl chloride (0.032 mL, 0.318 mmol) and 1 M sodium carbonate solution (1 mL). Preparative HPLC purification gave 86 mg (83% yield) of the title compound 25-14. ¹H NMR (CD₃OD): δ 7.65 (1H, br d), 7.33-7.17 (5H, m), 7.08 (1H, d, J=3.6 Hz), 6.57 (1H, dd, J=3.3, 1.5 Hz), 4.48 (1H, m), 4.05 (2H, m), 3.05 (1H, dd, J=13.6, 6.4 Hz), 2.94 (1h, dd, J=13.6, 8.1 Hz); HRMS (MALDI) calc for C₁₄H₁₇NO₆P (M+H⁺) 326.0794; found 326.0801.

### Example 25-15

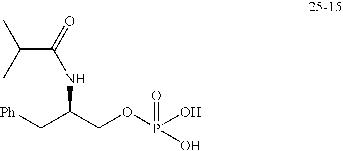

To a sodium carbonate solution (1 M, 1 mL) was added the aminophosphate 21a (106 mg, 0.397 mmol) and 2-methylpropanoic anhydride (0.16 mL, 153 mg, 0.966 mmol). After 15 h, it was acidified to pH˜1 by addition of concentrated HCl solution at 0° C. Preparative HPLC purification afforded 110 mg (92% yield) of the title compound 25-15. ¹H NMR (CD₃OD): δ 7.29-7.08 (5H, m), 6.94 (1H, br d), 4.34 (1H, br s), 4.10 (1H, m), 3.96 (1H, m), 2.82 (2H, m), 2.41 (1H, heat, J=7.2 Hz), 1.00 (3H, d, J=7 Hz), 0.97 (3H, d, J=7 Hz); LCMS (ESP): 302 (M+H⁺); 300 (M−H)⁻.

### Example 25-16

### Phosphoric acid mono-[(R)-2-(2,2-dimethyl-propanoylamino)-3-phenyl-propyl]ester

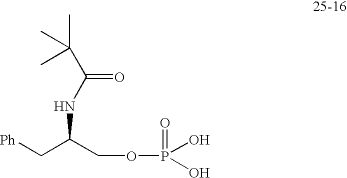

Prepared as described in the synthesis of 25-15 using 21a (110 mg, 0.412 mmol), 2,2-dimethylpropanoic anhydride (0.16 mL, 147 mg, 0.79 mmol) and 1 M sodium carbonate solution (1 mL). Preparative HPLC purification gave 130 mg (100% yield) of the title compound 25-16. ¹H NMR (CD₃OD): δ 7.32-7.11 (5H, m), 6.25 (1H, br d, J=8.7 Hz), 4.37 (1H, m), 4.13 (1H, m), 3.98 (1H, m), 2.93 (1H, dd, J=14.1, 6.6 Hz), 2.80 (1H, dd, J=14.1, 8.5 Hz), 1.06 (9H, s); LCMS (ESP): 316 (M+H⁺); 314 (M−H)⁻.

### Example 25-40

### Phosphoric acid mono-(2-acetylamino-3-phenyl-propyl)ester

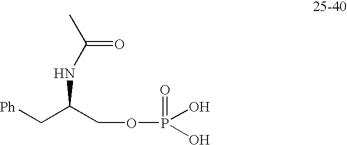

Prepared as described in the synthesis of 25-15 using 21a (120 mg, 0.45 mmol), acetic anhydride (0.1 mL, 108 mg, 1.0 mmol) and 1 M sodium carbonate solution (1 mL). Preparative HPLC purification gave 50 mg (40% yield) of the title compound 2540. ¹H NMR (CD₃OD): δ 7.64-7.30 (5H, m), 4.47 (1H, m), 4.16 (2H, m), 3.16 (1H, dd, J=14.1, 6.6 Hz), 2.99 (1H, dd, J=13.9, 8.7 Hz), 2.10 (3H, s); LCMS (ESP): 274 (M+H⁺), 296 (M+Na⁺); 272 (M−H)⁻.

### Example 25-17

### Phosphoric acid mono-{2-[(1-cyclohexyl-methanoyl)-amino]-3-phenyl-propyl}ester

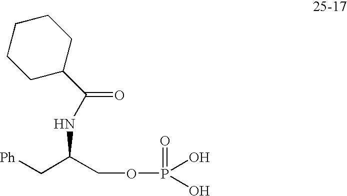

To an ether solution (5 mL) of cyclohexanecarboxylic acid (250 mg, 1.95 mmol) was added pyridine (0.5 mL) and cyclohexanecarbonyl chloride (286 mg, 0.261 mL, 1.95 mmol). After 10 h, the suspension was diluted with ether (20 mL), washed with ice-cold 5% HCl solution (1×50 mL) and concentrated NaHCO₃ solution (1×50 mL) and dried over Na₂SO₄. All solvent was removed in vacuo to give 350 mg of cyclohexanecarboxylic anhydride as a colorless oil. A portion of the cyclohexanecarboxylic anhydride (226 mg, 0.948 mmol) was added to a sodium carbonate solution (1 M, 2 mL) of the aminophosphate 21a (110 mg, 0.412 mmol). After 15 h, it was acidified to pH-1 by

addition of concentrated HCl solution at 0° C. Preparative HPLC purification afforded 50 mg (36% yield) of the title compound 25-17. ¹H NMR (CD₃OD): δ 7.27-7.1 (5H, m), 6.67 (1H, br d), 4.34 (1H, br s), 4.08 (1H, m), 3.96 (1H, m), 2.90 (1H, dd, J=14.4, 6.6 Hz), 2.78 (1H, dd, J=14.9, 9 Hz), 2.10 (1H, br t), 1.79-1.51 (5H, m), 1.33-1.02 (5H, m); LCMS (ESP): 342 (M+H⁺); 340 (M−H)⁻.

### Example 25-18

### Phosphoric acid mono-{2-[(1-1H-indol-2-yl-methanoyl)-amino]-3-phenyl-propyl}ester

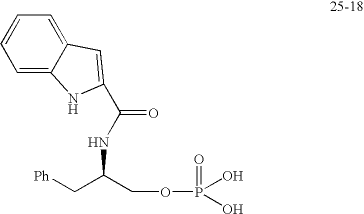

To a DMF solution (1 mL) of the aminophosphate 21a (78 mg, 0.292 mmol) was added imidazole (65 mg, 0.962 mmol) and t-butyldimethylchlorosilane (110 mg, 0.730 mmol). After 3.5 h, indole-2-carboxylic acid (49 mg, 0.307 mmol), EDC (70 mg, 0.365 mmol), DMAP (4-dimethylaminopyridine) (7 mg, 0.058 mmol) were added to the reaction. The mixture was stirred for 15 h and was acidified to pH˜1 by addition of concentrated HCl solution at 0° C. Preparative HPLC purification afforded 1 mg (1% yield) of the title compound 25-18. ¹H NMR (CD₃OD), δ 7.48 (1H, d, J=8.5 Hz), 7.30 (1H, d, J=8.4 Hz), 7.25-6.89 (7H, m), 4.36 (1H, m), 3.93 (2H, m), 2.92 (2H, m); LCMS (ESP): 373 (M−H)⁻.

### Example 25-19

### Phosphoric acid mono-{2-[(1-benzofuran-2-yl-methanoyl)-amino]-3-phenyl-propyl}ester

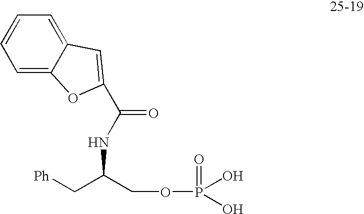

Prepared as described in the synthesis of 25-18 using aminophosphate 21a (94 mg, 0.351 mmol), imidazole (79 mg, 1.16 mmol), t-butyidimethylchlorosilane (132 mg, 0.878 mmol), 1-benzofuran-2-carboxylic acid (60 mg, 0.369 mmol), EDC (84 mg, 0.439 mmol) and DMAP (9 mg, 0.07 mmol). Preparative HPLC purification gave 16 mg (12% yield) of the title compound 25-19. ¹H NMR (CD₃OD): δ 7.59 (1H, d, J=7.8 Hz), 7.47 (1H, d, J=8.1 Hz), 7.39-7.29 (2H, m), 7.25-7.03 (6H, m), 4.43 (1H, m), 3.98 (2H, m), 2.96 (1H, dd, J=13.8, 6.2 Hz), 2.87 (1H, dd, J=13.8, 8.1 Hz); LCMS (ESP): 374 (M−H)⁻.

### Example 25-20

### Phosphoric acid mono-(2-{[1-(6-hydroxy-naphthalen-2-yl)-methanoyl]-amino}-3-phenyl-propyl)ester

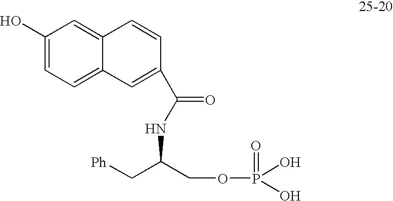

Prepared as described in the synthesis of 25-18 using aminophosphate 21a (152 mg, 0.568 mmol), imidazole (155 mg, 2.27 mmol), t-butyidimethylchlorosilane (214 mg, 1.42 mmol), 6-(acetyloxy)-2-naphthoic acid (131 mg, 0.569 mmol), EDC (136 mg, 0.71 mmol) and DMAP (14 mg, 0.114 mmol). Preparative HPLC purification gave 25 mg (11% yield) of the title compound 25-20. ¹H NMR (CD₃OD): δ 8.21 (1H, s), 7.9-7.6 (4H, m), 7.4-7.05 (6H, m), 4.57 (1H, m), 4.12 (2H, m), 3.00 (2H, m); LCMS: 400 (M−H)⁻.

### Example 25-21

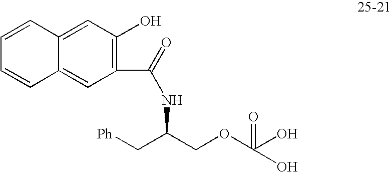

Prepared as described in the synthesis of 25-18 using aminophosphate 21a (94 mg, 0.351 mmol), imidazole (96 mg, 1.4 mmol), t-butyldimethylchlorosilane (132 mg, 0.878 mmol), 1-hydroxy-2-naphthoic acid (66 mg, 0.351 mmol), EDC (84 mg, 0.439 mmol) and DMAP (9 mg, 0.07 mmol). Preparative HPLC purification gave 7 mg (5% yield) of the title compound 25-21. ¹H NMR (CD₃OD): δ 8.24 (1H, d, J=8.1 Hz), 7.70 (1H, d, J=7.8 Hz), 7.64 (1H, d, J=9 Hz), 7.49 (1H, t, J=7.2 Hz), 7.41 (1H, d, J=8.1 Hz), 7.3-7.05 (6H, m), 4.50 (1H, m), 4.04 (2H, m), 2.98 (2H, m); HRMS (MALDI) calc for C₂₀H₂₁NO₆P (M+H⁺) 402.1107; found 402.1099.

### Example 25-22

### Phosphoric acid mono-(2-{[1-(3-hydroxy-naphthalen-2-yl)-methanoyl]-amino}-3-phenyl-propyl)ester

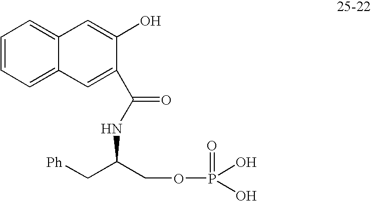

Prepared as described in the synthesis of 25-18 using aminophosphate 21a (98 mg, 0.366 mmol), imidazole (100 mg, 1.46 mmol), t-butyldimethylchlorosilane (138 mg, 0.915 mmol), 3-hydroxy-2-naphthoic acid (69 mg, 0.366 mmol), EDC (88 mg, 0.458 mmol) and DMAP (9 mg, 0.0732 mmol). Preparative HPLC purification gave 2 mg (1% yield) of the title compound 25-22. ¹H NMR (CD₃OD): δ 8.31 (1H, s), 7.72 (1H, d, J=8.4 Hz), 7.55 (1H, d, J=8.7 Hz), 7.36 (1H, t, J=7.8 Hz), 7.28-7.02 (7H, m), 4.47 (1H, m), 4.00 (2H, m), 2.96 (2H, m); LCMS (ESP): 402(M+H⁺), 424 (M+Na⁺).

### Example 25-23

### Phosphoric acid mono-{(R)-2-[(1H-benzoimidazole-5-carbonyl)-amino]-3-phenyl-propyl}ester

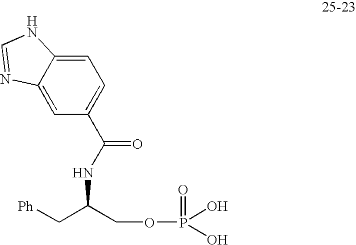

Prepared as described in the synthesis of 25-18 using aminophosphate 21a (100 mg, 0.374 mmol), imidazole (76 mg, 1.12 mmol), t-butyldimethylchlorosilane (141 mg, 0.935 mmol), 6-(acetyloxy)-2-naphthoic acid (72.7 mg, 0.449 mmol), EDC (86 mg, 0.449 mmol) and DMAP (10 mg, 0.081 mmol). Before the acidification the reaction mixture was treated with 10% NaOH solution (1 mL) for 10 h. Preparative HPLC purification gave 30 mg (21% yield) of the title compound 25-23. ¹H NMR (CD₃OD): δ 9.44 (1H, s), 8.23 (1H, s), 7.99 (1H, d, J=8.7 Hz), 7.87 (1H, d, J=8.7 Hz), 7.37-7.13 (5H, m), 4.55 (1H, m), 4.13 (2H, m), 3.09 (1H, dd, J=13.5, 6.9 Hz), 2.99 (1H, dd, J=13.5, 8.1 Hz); LCMS (ESP): 376 (M+H⁺); 374 (M−H)⁻.

### Example 25-24

### Phosphoric acid mono-(2-{[1-(1-bromo-naphthalen-2-yl)-methanoyl]amino}-3-phenyl-propyl)ester

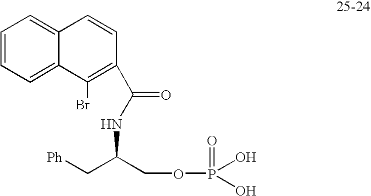

To a methylene chloride solution (2 mL) of 1-bromo-2-naphthoic acid (182 mg, 0.725 mmol) was added oxalyl chloride (0.19 mL, 2.18 mmol) and 2 drops of DMF. The mixture was stirred for 2 h and concentrated in vacuo. To the residue was added sodium carbonate solution (1 M, 2 mL), the aminophosphate 21a (194 mg, 0.725 mmol) and 1 mL of acetonitrile. After 15 h, it was acidified to pH˜1 by addition of concentrated HCl solution at 0° C. Preparative HPLC purification afforded 103 mg (31% yield) of the title compound 25-24. ¹H NMR (CD₃OD): δ 8.32 (1H, d, J=8.7 Hz), 7.92 (2H, m), 7.65 (2H, m), 7.41-7.20 (6H, m), 4.60 (1H, m), 4.14 (2H, m), 3.12 (1H, dd, J=13.9, 6 Hz), 2.94 (1H, dd, 13.8, 8.7 Hz); HRMS (MALDI) calc for C₂₀H₂₀NO₅PBr (M+H⁺) 464.0262; found 464.0271.

### Example 25-25

### Phosphoric acid mono-(2-{[1-(6-methoxy-naphthalen-2-yl)-methanoyl]-amino}-3-phenyl-propyl)ester

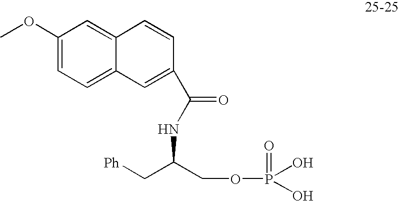

Prepared as described in the synthesis of 25-24 using 6-methoxy-2-naphthoic acid (255 mg, 1.26 mmol), oxalyl chloride (0.33 mL, 3.78 mmol), DMF (2 drops), sodium carbonate solution (1 M, 2 mL) and the aminophosphate 21a (337 mg, 1.26 mmol). Preparative HPLC purification afforded 44 mg (13% yield) of the title compound 25-25. ¹H NMR (CD₃OD): δ 8.24 (1H, s), 7.86 (1H, d, J=9.3 Hz), 7.81 (2H, m), 7.41 (7H, m), 4.56 (1H, m), 4.13 (2H, m), 3.95 (3H, s), 3.11 (1H, dd, J=14.1, 7.2 Hz), 3.01 (1H, dd, J=13.6, 8.5 Hz); LCMS (ESP): 414 (M−H); Elemental Analysis for (C₂₁H₂₂NO₆P 0.25H₂O) calc: C, 60.07; H, 5.40; N, 3.34; found: C, 59.66; H, 5.33; N, 3.74.

### Example 25-26

### Phosphoric acid mono-{2-[(1-benzo[b]thiophen-2-yl-methanoyl)-amino]-3-phenyl-propyl}ester

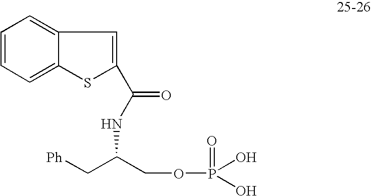

To a sodium carbonate solution (1 M, 1 mL) was added the aminophosphate 21b (68 mg, 0.254 mmol) and benzothiophene-2-carbonyl chloride (50 mg, 0.254 mmol). After 15 h, it was acidified to pH˜1 by addition of concentrated HCl solution at 0° C. Preparative HPLC purification afforded 20 mg (20% yield) of the title compound 25-26. ¹H NMR (CD₃OD): δ 7.99-7.86 (3H, m), 7.53-7.15 (7H, m), 4.49 (1H, m), 4.10 (2H, m), 3.04 (2H, m); LCMS (ESP): 414 (M+Na⁺); 390 (M−H)⁻.

### Example 25-27

### Phosphoric acid mono-{2-[(1-naphthalen-2-yl-methanoyl)-amino]-3-phenyl-propyl}ester

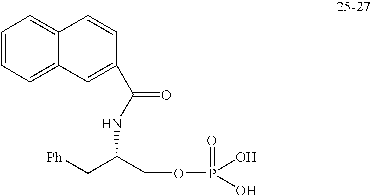

Prepared as described in the synthesis of 25-26 using 21b (70 mg, 0.262 mmol), 2-naphthoyl chloride (50 mg, 0.262 mmol) and 1 M sodium carbonate solution (1 mL). Preparative HPLC purification gave 20 mg (20% yield) of the title compound 25-27. ¹H NMR (CD₃OD): δ 8.32 (1H, s), 8.08-7.78 (4H, m), 7.66-7.5 (2H, m), 7.39-7.17 (5H, m), 4.56 (1H, m), 4.11 (2H, m), 3.12 (1H, dd, J=13.7, 6.8 Hz), 3.02 (1H, dd, J=13.8, 7.7 Hz); LCMS (ESP): 386 (M+H⁺), 408 (M+Na⁺); 384 (M−H)⁻.
Aminoalcohol 18c:

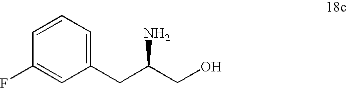

To a THF solution (30 mL) of D-3-fluorophenylalanine (5 g, 27.3 mmol) at 0° C. was added borane in THF (1 M, 68.3 mL, 68.3 mmol). After 5 h at 25° C., to the solution was added sat'd NaHCO₃ solution (10 mL), which was stirred for 15 h. The mixture was concentrated and extracted with methylene chloride (3×50 mL). Combined organic layers were dried (Na₂SO₄) and concentrated. The residue was purified by column chromatography (CH₂Cl₂/MeOH/NH₄OH 95/5/0.5 to 90/10/1) to give 1.94 g (42% yield) of the compound 18c as a white solid. ¹H NMR (CD₃OD): δ 7.32-7.21 (1H, m), 7.00-6.98 (3H, m), 3.63 (1H, dd, J=11.1, 4.2 Hz), 3.38 (1H, dd, J=10.5, 6.9 Hz), 3.12 (1H, m), 2.79 (1H, dd, J=13.5, 5.1 Hz), 2.54 (1H, dd, J=13.5, 8.4 Hz); LCMS (ESP): 170 (M+H⁺).
Alcohol 19c:

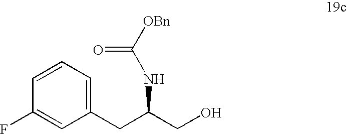

To a methylene chloride solution (30 mL) of aminoalcohol 18c (1.94 g, 11.5 mmol) was added triethylamine (2.8 mL, 20 mmol) and benzyl chloroformate (2.05 mL, 14.3 mmol). The mixture was stirred for 15 h and then diluted with methylene chloride (50 mL). The solution was washed with brine (1×50 mL), dried (Na₂SO₄) and concentrated. After column chromatography purification (20 to 30% EtOAc in hexanes), the compound 19c was obtained in 64% yield (2.22 g). ¹H NMR (CDCl₃): δ 7.4-7.19 (6H, m), 7.02-6.86 (3H, m), 5.08 (2H, s), 4.95 (1H, br s), 3.93 (1H, m), 3.69 (1H, dd, J=10.2, 3 Hz), 3.58 (1H, dd, J=11.4, 4.8 Hz), 2.87 (2H, d, J=7.5 Hz); LCMS (ESP): 326 (M+Na⁺); Elemental Analysis for (C₁₇H₁₈FNO₃) calc: C, 67.31; H, 5.98; N, 4.62; found: C, 67.31; H, 5.98; N, 4.62.
Phosphate Benzyl Ester 20c:

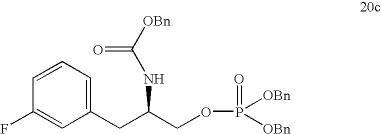

To an acetonitrile solution (40 mL) of the alcohol 19c (2.21 g, 7.29 mmol) and 1H-tetrazole (1.37 g, 19.6 mmol) was added dibenzyl N,N-diisopropylphosphoramidite (4.9 mL, 14.6 mmol) at 25° C. After 5 h, MCPBA (5.72 g, 77% pure, 25.5 mmol) was added to the suspension. The solution was diluted with methylene chloride (100 mL), washed with concentrated NaHSO₃ solution (2×80 mL), dried over MgSO₄ and concentrated in vacuo. The residue was purified by column chromatography (15-30% EtOAc in hexanes) to give 4.39 g of the compound 20c in 100% yield. ¹H NMR (CDCl₃): δ 7.44-7.12 (16H, m), 6.96-6.76 (3H, m), 5.18-4.96 (7H, m), 4.04-3.78 (3H, m), 2.87-2.63 (2H, m).
Aminophosphate 21c:

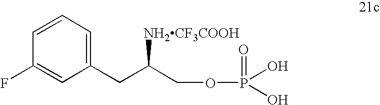

To an ethanol solution of the phosphate benzyl ester (20c, 4.37 g, 7.76 mmol) was added palladium on carbon (10%, 870 mg). The suspension was kept under hydrogen atmosphere (1 atm) for 15 h and was then added 5% HCl solution (10 mL). The mixture was filtered through a pad of celite. The filtrate was concentrated to dryness, affording 2.30 g of a yellowish solid. Preparative HPLC purification gave 1.2 g (62% yield) of the compound 21c as a white solid. ¹H NMR (CD₃OD): δ 7.45-7.35 (1H, m), 7.18-7.01 (3H, m), 4.10 (1H, m), 3.94 (1H, m), 3.70 (1H, m), 3.03 (2H, m); LCMS (ESP): 250 (M+H⁺); 248 (M−H)⁻.

### Example 25-28

### Phosphoric acid mono-{3-(3-fluoro-phenyl)-2-[(1-naphthalen-2-yl-methanoyl)-amino]-propyl}ester

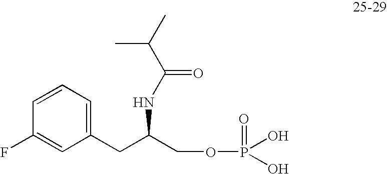

To a sodium carbonate solution (1 M, 1 mL) was added the aminophosphate 21c (110 mg, 0.385 mmol) and 2-naphthoyl chloride (110 mg, 0.578 mmol). After 15 h, it was acidified to pH-1 by addition of concentrated HCl solution at 0° C. Preparative HPLC purification afforded 160 mg (100% yield) of the title compound 25-28. ¹H NMR (CD₃OD): δ 8.31 (1H, s), 8.0-7.88 (3H, m), 7.82 (1H, dd, J=8.7, 1.8 Hz), 7.58 (2H, m), 7.31 (1H, m), 7.14 (2H, m), 6.95 (1H, br td), 4.59 (1H, m), 4.14 (2H, m), 3.13 (1H, dd, J=13.8, 6 Hz), 3.02 (1H, dd, J=13.9, 8.7 Hz); HRMS (MALDI) calc for C₂₀H₂₀NO₅ PF (M+H⁺) 404.1063; found 404.1078.

### Example 25-29

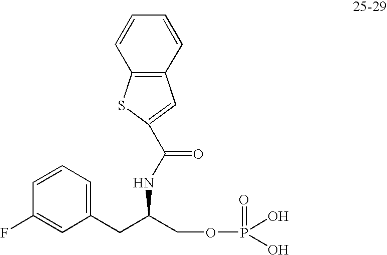

Prepared as described in the synthesis of 25-28 using 21c (110 mg, 0.386 mmol), 1-benzothiophene-2-carbonyl chloride (114 mg, 0.578 mmol) and 1 M sodium carbonate solution (1 mL). Preparative HPLC purification gave 145 mg (92% yield) of the title compound 25-29. ¹H NMR (CD₃OD): δ 7.95 (1H, s), 7.93-7.85 (2H, m), 7.4 (2H, m), 7.30 (1H, m), 7.2-7.04 (2H, m), 6.94 (1H, br td), 4.51 (1H, m), 4.2-4.0 (2H, m), 3.11 (1H, dd, J=13.9, 6.4 Hz), 3.01 (1H, dd, J=13.9, 8.3 Hz); HRMS (MALDI) calc for C₁₈H₁₈NO₅ PFS (M+H⁺) 410.0627; found 410.0639.

### Example 25-30

### Phosphoric acid mono-[(R)-2-(2,2dimethyl-propanoylamino)-3-(3-fluoro-phenyl)-propyl]ester

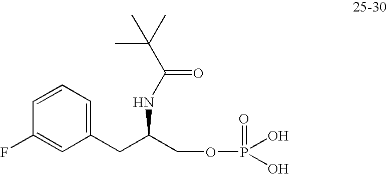

To a sodium carbonate solution (1 M, 1 mL) was added the aminophosphate 21c (65 mg, 0.228 mmol) and 2,2-dimethylpropanoic anhydride (0.08 mL, 0.392 mmol). After 15 h, it was acidified to pH˜1 by addition of concentrated HCl solution at 0° C. Preparative HPLC purification afforded 43 mg (57% yield) of the title compound 25-30. ¹H NMR (CD₃OD): δ 7.19 (1H, m), 7.0-6.77 (3H, m), 4.23 (1H, m), 3.89 (2H, m), 2.91 (1H, dd, J=13.8, 5.5 Hz), 2.74 (1H, dd, J=13.8, 9.2 Hz), 1.00 (9H, s); HRMS (MALDI) calc for C₁₄H₂₂NO₅ PF (M+H⁺) 334.1220; found 334.1223.

### Example 25-31

### Phosphoric acid mono-[(R)-2-{[1-(1-bromo-naphthalen-2-yl)-methanoyl]-amino}-3-(3-fluoro-phenyl)-propyl]ester

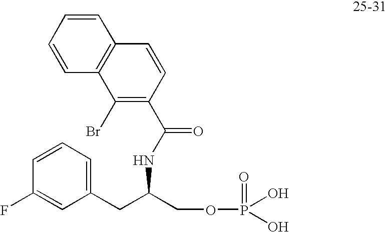

To a methylene chloride solution (3 mL) of 1-bromo-2-naphthoic acid (161 mg, 0.643 mmol) was added oxalyl chloride (0.168 mL, 1.93 mmol) and 2 drops of DMF. The mixture was stirred for 2 h and concentrated in vacuo. To the residue was added sodium carbonate solution (1 M, 2 mL), the aminophosphate 21c (80 mg, 0.28 mmol) and 1 mL of acetonitrile. After 15 h, it was acidified to pH-1 by addition of concentrated HCl solution at 0° C. Preparative HPLC purification afforded 70 mg (52% yield) of the title compound 25-31. ¹H NMR (CD₃OD): δ 8.22 (1H, d, J=8.7 Hz), 7.84 (1H, m), 7.56 (2H, m), 7.27 (1H, m), 7.18 (1H, d, J=8.7 Hz), 7.09 (1H, d, J=7.8 Hz), 7.03 (1H, m), 6.91 (1H, td, J=8.7, 3 Hz), 4.51 (1H, m), 4.04 (2H, m), 3.05 (1H, dd, J=14.1, 5.7 Hz), 2.84 (1H, dd, 14.1, 9.3 Hz); LCMS (ESP): 481 (M−H)⁻; Elemental Analysis for (C₂₀H₁₈BrFNO₅P) calc: C, 49.81; H, 3.76; N, 2.90; found: C, 49.63; H, 3.73; N, 2.92.

### Example 25-32

### Phosphoric acid mono-((R)-3-(3-fluoro-phenyl)-2-{[1-(6-methoxy-naphthalen-2-yl)-methanoyl]-amino}-propyl)ester

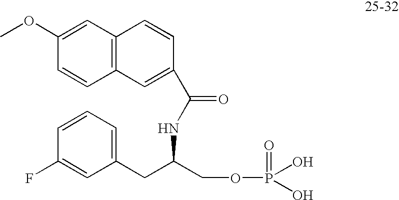

Prepared as described in the synthesis of 25-31 using 6-methoxy-2-naphthoic acid (143 mg, 7.07 mmol), oxalyl chloride (0.185 mL, 2.12 mmol), DMF (2 drops), sodium carbonate solution (1 M, 2 mL) and the aminophosphate 21c (88 mg, 0.353 mmol). Preparative HPLC purification afforded 50 mg (39% yield) of the title compound 25-32. ¹H NMR (CD₃OD): δ 8.25 (1H, s), 7.9-7.73 (3H, m), 7.37-7.06 (5H, m), 6.95 (1H, t, J=8.1 Hz), 4.57 (1H, m), 4.12 (2H, m), 3.95 (3H, s), 3.13 (1H, dd, J=13.8, 6.6 Hz), 3.01 (1H, dd, J=14.3, 7.7 Hz); LCMS (ESP): 432 (M−H)⁻; Elemental Analysis for (C₂₁H₂₁FNO₆P 0.25H₂O) calc: C, 57.60; H, 4.95; N, 3.20; found: C, 57.58; H, 4.97; N, 3.27.
Aminoalcohol 18d:

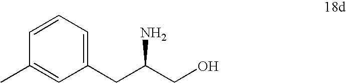

To a THF solution (10 mL) of D-3-methylphenylalanine (1 g, 5.58 mmol) at 0° C. was added borane in THF (1 M, 22.4 mL, 22.4 mmol). After 48 h at 25° C., the solution was added sat'd NaHCO₃ solution (2 mL) and stirred for 3 h. The mixture was concentrated and extracted with methylene chloride (3×20 mL). Combined organic layers were dried (Na₂SO₄) and concentrated. The residue was purified by column chromatography (CH₂Cl₂/MeOH/NH₄OH 95/5/0.5 to 90/10/1) to give 490 mg (53% yield) of the compound 18d as a white solid. ¹H NMR (CD₃OD): δ 7.20 (1H, t, J=7.8 Hz), 7.08-6.94 (3H, m), 3.63 (1H, dd, J=10.5, 3.9 Hz), 3.37 (1H, dd, J=10.5, 7.5 Hz), 3.11 (1H, m), 2.76 (1H, dd, J=13.5, 5.7 Hz), 2.49 (1H, dd, J=13.8, 8.4 Hz), 2.34 (3H, s); LCMS (ESP): 166 (M+H⁺).
Alcohol 19d:

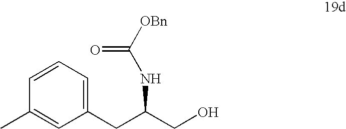

To a methylene chloride solution (5 mL) of aminoalcohol 18d (150 mg, 0.909 mmol) was added triethylamine (0.3 mL) and benzyl chloroformate (0.21 mL, 1.5 mmol). The mixture was stirred for 15 h and then diluted with methylene chloride (20 mL). The solution was washed with brine (1×20 mL), dried (Na₂SO₄) and concentrated. After column chromatography purification (15 to 30% EtOAc in hexanes), the compound 19d was obtained in 63% yield (170 mg). ¹H NMR (CDCl₃): δ 7.40-7.27 (5H, m), 7.18 (1H, t, J=7.8 Hz), 7.08-6.95 (3H, m), 5.09 (2H, s), 4.94 (1H, br s), 3.94 (1H, m), 3.69 (1H, br d), 3.58 (1H, dd, J=14.5, 5.4 Hz), 2.82 (2H, d, J=6.9 Hz), 2.32 (3H, s); LCMS (ESP): 322 (M+Na⁺).

### Phosphate Benzyl Ester 20d

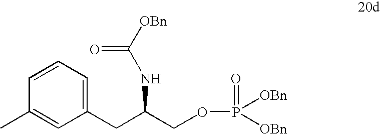

To an acetonitrile solution (8 mL) of the alcohol 19d (160 mg, 0.535 mmol) and 1H-tetrazole (101 mg, 1.44 mmol) was added dibenzyl N,N-diisopropylphosphoramidite (0.36 mL, 1.07 mmol) at 25° C. After 5 h, MCPBA (0.42 g, 77% pure, 1.87 mmol) was added to the suspension. The solution was diluted with methylene chloride (35 mL), washed with concentrated NaHSO₃ solution (2×25 mL), dried over MgSO₄ and concentrated in vacuo. The residue was purified by column chromatography (15-30% EtOAc in hexanes) to give 0.287 g of the compound 20d in 96% yield. ¹H NMR (CDCl₃): δ 7.39-7.2 (15H, m), 7.12 (1H, t, J=7.8 Hz), 7.01 (1H, d, J=7.8 Hz), 6.93 (2H, m), 5.14-4.98 (7H, m), 4.05-3.82 (3H, m), 2.78 (1H, br dd), 2.69 (1H, dd, J=113.8, 7.8 Hz), 2.27 (3H, s).
Aminophosphate 21d:

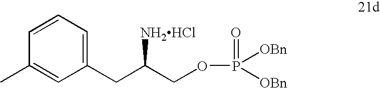

To an ethanol solution of the phosphate benzyl ester 20d (0.287 g, 0.513 mmol) was added palladium on carbon (10%, 58 mg). The suspension was kept under hydrogen atmosphere (1 atm) for 15 h and was then added 5% HCl solution (5 mL). The mixture was filtered through a pad of celite. The filtrate was concentrated to dryness, affording 134 mg (92% yield) of 21d a yellowish solid. ¹H NMR (CD₃OD): δ 7.26 (1H, t, J=7.5 Hz), 7.18-7.06 (3H, m), 4.12 (1H, br d), 3.99 (1H, m), 3.68 (1H, m), 2.98 (2H, d, J=7.8 Hz), 2.36 (3H, s).

### Example 25-33

### Phosphoric acid mono-[(R)-2-{[1-(diethylamino-oxo-2H-chromen-3-yl)-methanoyl]-amino}-3-(3-fluoro-phenyl)-propyl]ester

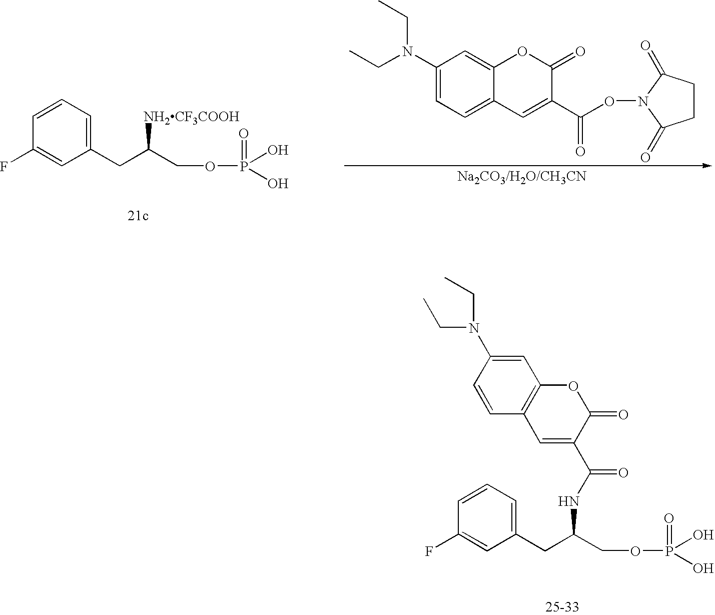

To a sodium carbonate solution (1 M, 2 mL) was added the aminophosphate 21c (50 mg, 0.176 mmol) and 7-diethylaminocoumarin-3-carboxylic acid, succinimidyl ester (50 mg, 0.140 mmol). After 15 h, it was acidified to pH˜1 by addition of 1 M HCl solution at 0° C. Preparative HPLC purification afforded 43 mg (57% yield) of the title compound 25-33. ¹H NMR (CD₃OD): δ 8.69 (1H, s), 7.54 (1H, d, J=9.04 Hz), 7.31 (1H, dd, J=14.12, 7.91 Hz), 7.12 (2H, dd, J=17.33, 9.80 Hz), 6.94 (1H, t, J=8.86 Hz), 6.83 (1H, dd, J=9.04, 2.44 Hz), 6.95 (1H, d, J=2.26 Hz), 4.50 (1H, m), 4.07 (2H, t, J=4.71 Hz), 3.54 (4H, m), 3.14-2.92 (2H, m), 1.25 (6H, t, J=7.16 Hz); HRMS (MALDI) calc for C₂₃H₂₆FN₂O₇PH (M+H⁺) 493.1549; found 493.1540.

### Example 25-34

### Phosphoric acid mono-((R)-2-{[1-(1-bromo-naphthalen-2-yl)-methanoyl]-amino}-3-m-tolyl-propyl)ester

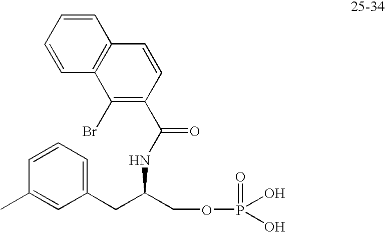

To a methylene chloride solution (3 mL) of 1-bromo-2-naphthoic acid (123 mg, 0.49 mmol) was added oxalyl chloride (0.128 mL, 1.47 mmol) and 2 drops of DMF. The mixture was stirred for 3.5 h and concentrated in vacuo. To the residue was added sodium carbonate solution (1 M, 2 mL), the aminophosphate 21d (69 mg, 0.245 mmol) and 2 mL of acetonitrile. After 15 h, it was acidified to pH˜1 by addition of concentrated HCl solution at 0° C. Preparative HPLC purification afforded 32 mg (27% yield) of the title compound 25-34. ¹H NMR (CD₃OD): δ 8.22 (1H, d, J=8.4 Hz), 7.82 (2H, m), 7.7.6-7.46 (2H, m), 7.2-6.9 (5H, m), 4.48 (1H, m), 4.03 (2H, m), 2.98 (1H, dd, J=13.9, 6.2 Hz), 2.80 (1H, dd, J=13.9, 8.7 Hz), 2.26 (3H, s); HRMS (MALDI) calc for C₂₁H₂₂NO₅PBr (M+H⁺) 478.0419; found 478.0438.

### Example 25-35

### Phosphoric acid mono-((R)-2-{[1-(6-methoxy-naphthalen-2-yl)-methanoyl]-amino}-3-m-tolyl-propyl)ester

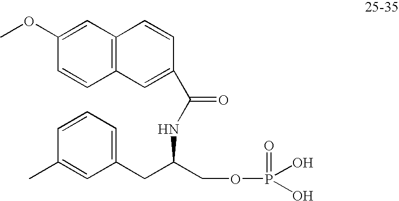

Prepared as described in the synthesis of 25-32 using 6-methoxy-2-naphthoic acid (99 mg, 0.49 mmol), oxalyl chloride (0.128 mL, 1.47 mmol), DMF (2 drops), sodium carbonate solution (1 M, 2 mL) and the aminophosphate 21d (69 mg, 0.245 mmol). Preparative HPLC purification afforded 76 mg (51% yield) of the title compound 25-35. ¹H NMR (CD₃OD): δ 8.24 (1H, s), 7.86 (1H, d, J=8.7 Hz), 7.82 (2H, m), 7.30 (1H, d, J=2.4 Hz), 7.24-7.09 (4H, m), 7.03 (1H, d, J=7.5 Hz), 4.53 (1H, m), 4.10 (2H, m), 3.95 (3H, s), 3.06 (1H, dd, J=13.9, 6.8 Hz), 2.96 (1H, dd, J=14, 8.3 Hz), 2.31 (3H, s); HRMS (MALDI) calc for C₂₂H₂₅NO₆P (M+H⁺) 430.1420; found 430.1436.
Alcohol 26a:

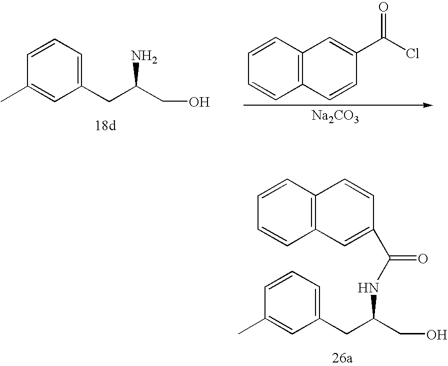

To a sodium carbonate solution (1 M, 2 mL) was added acetonitrile (1 mL), the aminoalcohol 18d (180 mg, 1.09 mmol) and 2-naphthoyl chloride (250 mg, 1.31 mmol). After 10 h, the mixture was extracted with methylene chloride (2×20 mL). Combined organic layers were dried (MgSO₄) and concentrated. The residue was purified by column chromatography (40% EtOAc in hexanes) to afford 350 mg (100% yield) of the compound 26a. ¹H NMR (CD₃OD): δ 8.07 (1H, s), 7.82-7.73 (3H, m), 7.65 (1H, dd, J=9, 1.5 Hz), 7.54-7.41 (2H, m), 7.16 (1H, t, J=7.2 Hz), 7.08-6.97 (3H, m), 6.46 (1H, br d, J=7.2 Hz), 4.33 (1H, m), 3.77 (1H, dd, J=11.1, 3.3 Hz), 3.68 (1H, dd, J=11.1, 5.1 Hz), 2.93 (2H, d, J=7.2 Hz), 2.27 (3H, s).
Alcohol 26b:

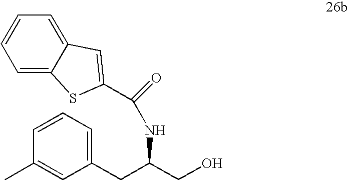

Prepared as described in the synthesis of the alcohol 26a using the aminoalcohol 18d (131 mg, 0.794 mmol), 1-benzothiophene-2-carbonyl chloride (195 mg, 0.992 mmol) in a co-solvent of sodium carbonate solution (1 M, 1.5 mL) and acetonitrile (1.5 mL). Purification by column chromatography (50% EtOAc in hexanes) afforded 230 mg (89% yield) of the compound 26b. ¹H NMR (CD₃OD): δ 7.96-7.81 (3H, m), 7.50-7.36 (2H, m), 7.21-7.04 (3H, m), 7.01 (1H, d, J=7.5 Hz), 4.32 (1H, m), 3.67 (1H, d, J=5.4 Hz), 3.00 (1H, dd, J=13.1, 6.6 Hz), 2.87 (1H, dd, J=13.1, 8.7 Hz), 2.29 (3H, s); LCMS (ESP): 326 (M+H⁺), 348 (M+Na⁺); 324 (M−H)⁻.
Phosphate Benzyl Ester 27a:

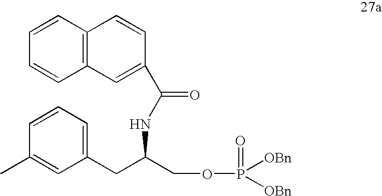

To an acetonitrile solution (10 mL) of the alcohol 26a (348 mg, 1.09 mmol) and 1H-tetrazole (191 mg, 2.728 mmol) was added dibenzyl N,N-diisopropylphosphoramidite (0.92 mL, 941 mg, 2.728 mmol) at 25° C. After 5 h, MCPBA (0.938 g, 60% pure, 3.27 mmol) was added to the suspension. The solution was diluted with methylene chloride (35 mL), washed with concentrated NaHSO₃ solution (2×25 mL), dried over MgSO₄ and concentrated in vacuo. The residue was purified by column chromatography (30% EtOAc in hexanes) to give 0.400 g of the compound 27a in 63% yield. ¹H NMR (CDCl₃): δ 8.34 (1H, s), 7.95-7.82 (3H, m), 7.55 (2H, m), 7.41-7.28 (11H, m), 7.17 (1H, t, J=7.5 Hz), 7.10-6.99 (3H, m), 5.15-4.97 (5H, m), 4.50 (1H, m), 4.17-3.96 (2H, m), 3.04 (1H, dd, J=13.5, 5.4 Hz), 2.81 (1H, dd, J=13.5, 9 Hz), 2.31 (3H, s).
Phosphate Benzyl Ester 27b:

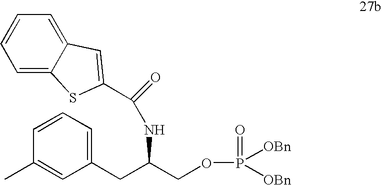

Prepared as described in the synthesis of compound 27a using the alcohol 26b (221 mg, 0.68 mmol), 1H-tetrazole (129 mg, 1.84 mmol), dibenzyl N,N-diisopropylphosphoramidite (0.46 mL, 1.36 mmol) and MCPBA (0.533 g, 77% pure, 2.38 mmol). Purification by column chromatography (30% EtOAc in hexanes) gave 0.50 g of the compound 27b in 100% yield. ¹H NMR (CDCl₃): δ 7.89-7.78 (3H, m), 7.5-7.29 (13H, m), 7.16 (1H, t, J=7.8 Hz), 7.08-6.95 (3H, m), 5.16-4.97 (4H, m), 4.41 (1H, m), 4.04 (2H, m), 3.05 (1H, dd, J=13.5, 5.4 Hz), 2.77 (1H, dd, J=13.5, 9.6 Hz), 2.31 (3H, s).

### Example 25-36

### Phosphoric acid mono-{2-[(1-naphthalen-2-yl-methanoyl)-amino]-3-m-tolyl-propyl}ester

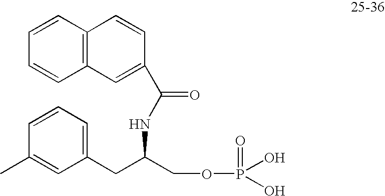

To an ethanol solution of the phosphate benzyl ester 27a (0.35 g, 0.604 mmol) was added palladium on carbon (10%, 35 mg). The suspension was kept under hydrogen atmosphere (1 atm) for 15 h and was then filtered through a pad of celite. All solvent was removed in vacuo. The residue was purified by preparative HPLC to give 45 mg (19% yield) of the title compound 25-36. ¹H NMR (CD₃OD): δ 8.21 (1H, s), 7.9-7.76 (3H, m), 7.72 (1H, dd, J=8.4, 1.8 Hz), 7.48 (2H, m), 7.13-7.0 (3H, m), 6.93 (1H, d, J=6.9 Hz), 4.45 (1H, m), 4.03 (2H, m), 2.97 (1H, dd, J=13.7, 6.8 Hz), 2.87 (1H, dd, J=13.5, 8.1 Hz); HRMS (MALDI) calc for C₂₁H₂₃NO₅P (M+H⁺) 400.1314; found 400.1314.

### Example 25-37

### Phosphoric acid mono-{2-[(1-benzo[b]thiophen-2-yl-methanoyl)-amino]-3-m-tolyl-propyl}ester

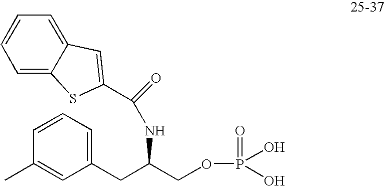

Prepared as described in the synthesis of 25-36 using the phosphate benzyl ester (27b, 0.398 g, 0.68 mmol) was added palladium on carbon (10%, 100 mg). The suspension was kept under hydrogen atmosphere (1 atm) for 72 h and was then filtered through a pad of celite. All solvent was removed in vacuo. The residue was purified by preparative HPLC to give 120 mg (44% yield) of the title compound 25-37. ¹H NMR (CD₃OD): δ 8.0-7.8 (3H, m), 7.44 (2H, m), 7.23-7.0 (4H, m), 4.47 (1H, m), 4.09 (2H, m), 3.04 (1H, dd, J=13.8, 6.8 Hz), 2.95 (1H, dd, J=14.1, 7.7 Hz), 2.30 (3H, s); LCMS (ESP): 404 (M−H)⁻; Elemental Analysis for (C₁₉H₂₀NO₅PS) calc: C, 56.29; H, 4.97; N, 3.46; found: C, 56.03; H, 4.94; N, 3.39.
Alcohol 28:

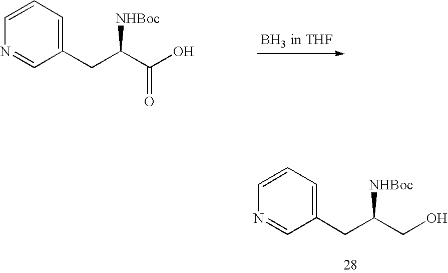

To a THF solution (10 mL) of Boc-D-Ala (3-pyridyl)-OH (4.39 g, 16.5 mmol) at 0° C. was added borane in THF (1 M, 40 mL, 40 mmol). After 20 h at 25° C., saturated NaHCO₃ solution (5 mL) was added to the solution and stirred for 3 h. The mixture was concentrated and extracted with methylene chloride (3×50 mL). Combined organic layers were dried (Na₂SO₄) and concentrated. The residue was purified by column chromatography (50% to 100% EtOAc in hexanes) to give 1.51 g (36% yield) of the compound 28 as a white solid. ¹H NMR (CDCl₃): δ 8.47 (2H, s), 7.84 (1H, d, J=7.8 Hz), 7.44 (1H, dd, J=7.8, 5.7 Hz), 4.82 (1H, m), 3.87 (1H, m), 3.72 (1H, dd, J=10.5, 3.6 Hz), 3.62 (1H, dd, J=10.5, 4.2 Hz), 2.95 (2H, m), 1.39 (9H, m); LCMS (ESP): 253 (M+H⁺), 275 (M+Na⁺); HRMS (MALDI) calc for C₁₃H₂₁N₂O₃ (M+H⁺) 253.1552; found 253.1564.
Phosphate Benzyl Ester 29:

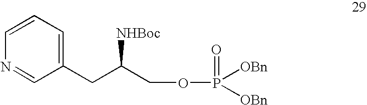

To an acetonitrile solution (15 mL) of the alcohol 28 (997 mg, 3.96 mmol) and 1H-tetrazole (1.11 g, 15.8 mmol) was added dibenzyl N,N-diisopropylphosphoramidite (2.66 mL, 7.91 mmol) at 25° C. After 3 h, MCPBA (2.67 g, 77% pure, 11.9 mmol) was added to the suspension. The solution was diluted with methylene chloride (60 mL), washed with concentrated NaHSO₃ solution (2×25 mL), dried over MgSO₄ and concentrated in vacuo. The residue was purified by column chromatography (50% to 100% EtOAc in hexanes) to give 0.481 g of the compound 29 in 24% yield. ¹H NMR (CDCl₃): δ 8.46 (1H, br d), 8.39 (1H, br s), 7.48 (1H, d, J=6.6 Hz), 7.36 (11H, m), 5.06 (4H, m), 4.81 (1H, m), 4.0-3.80 (2H, m), 2.72 (2H, d, J=6.3 Hz), 1.37 (9H, s); LCMS (ESP): 513 (M+H⁺); 535 (M+Na⁺).

### Example 30

### (1-Phosphonooxymethyl-2-pyridin-3-yl-ethyl)-carbamic acid tert-butyl ester

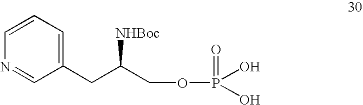

To an ethanol solution (3 mL) of the benzyl ester 29 (48 mg, 0.094 mmol) was added palladium on carbon (10%, 15 mg). The suspension was kept under hydrogen atmosphere (1 atm) for 15 h. After filtration, the filtrate was concentrated to dryness affording 36 mg (100% yield) of the title compound 30. ¹H NMR (CD₃OD): δ8.58 (1H, s), 8.49 (1H, brd), 8.19 (1H, d, J=7.5 Hz), 7.69 (1H, br t), 3.87 (1H, m), 3.79 (2H, m), 3.06 (1H, dd, J=13.9, 5.3 Hz), 2.82 (1H, dd, J=113.8, 8.5 Hz), 1.23 (9H, s); HRMS (MALDI) calc for C₁₃H₂₂N₂O₆P (M+H⁺) 333.1216; found 333.1218.
Aminophosphate 21e:

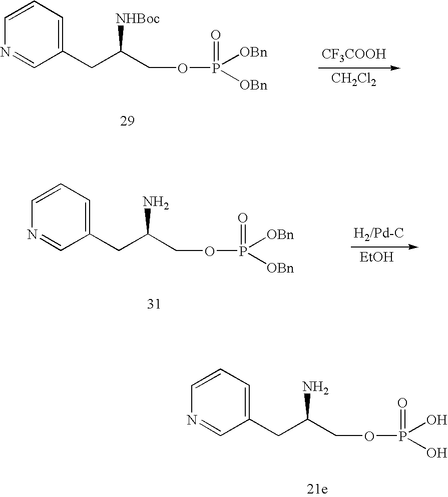

To a methylene chloride solution (2.5 mL) of the benzyl ester 29 (278 mg, 0.543 mmol) at 0° C. was added trifluoroacetic acid (0.75 mL). After 45 min, the solution was concentrated in vacuo to give 385 mg of amine 31 as a colorless oil. The crude amine 31 was dissolved in ethanol (5 mL) and added palladium on carbon (10%, 75 mg). The suspension was kept under hydrogen atmosphere (1 atm) for 48 h. After filtering, the filtrate was concentrated to dryness affording the compound 21e as a solid (225 mg, 100%). ¹H NMR (CD₃OD): δ 8.68 (1H, br s), 8.27 (1H, br s), 7.48 (1H, m), 7.25 (1H, m), 4.83 (1H, d), 4.0-3.6 (2H, m), 3.26-3.01 (2H, m); LCMS (ESP): 233 (M+H⁺), 465 (2M+H⁺); 231 (M−H)⁻.

### Example 25-38

### Phosphoric acid mono-{2-[(1-benzo[b]thiophen-2-yl-methanoyl)-amino]-3-pyridin-3-yl-propyl}ester

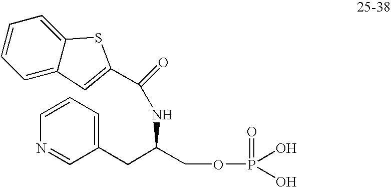

To a sodium carbonate solution (1 M, 1 mL) was added the aminophosphate 21e (110 mg, 0.474 mmol), 1-benzothiophene-2-carbonyl chloride (93 mg, 0.474 mmol). After 15 h, it was acidified to pH˜1 by addition of concentrated HCl solution at 0° C. Preparative HPLC purification afforded 25 mg (14% yield) of the title compound 25-38. ¹H NMR (CD₃OD): δ 8.83 (1H, s), 8.71-8.63 (1H, m), 8.58-8.46 (10H, m), 8.02-7.83 (4H, m), 7.44 (2H, m), 4.60 (1H, m), 4.17 (2H, m), 3.42-3.13 (2H, m); HRMS (MALDI) calc for C₁₇H₁₈N₂O₅PS (M+H⁺) 393.0674; found 393.0681.

### Example 25-39

### Phosphoric acid mono-{2-[(1-naphthalen-2-yl-methanoyl)-amino]-3-pyridin-3-yl-propyl}ester

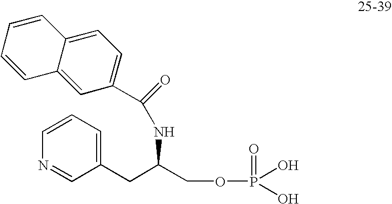

Prepared as described in the synthesis of 25-34 using 21e (97 mg, 0.418 mmol), 2-naphthoyl chloride (80 mg, 0.418 mmol) and 1 M sodium carbonate solution (2 mL). Preparative HPLC purification gave 25 mg (16% yield) of the title compound 25-39. ¹H NMR (CD₃OD): δ 8.85 (1H, s), 8.70 (1H, m), 8.58 (1H, d, J=8.4 Hz), 8.323 (1H, s), 8.0-7.85 (4H, m), 7.79 (1H, dd, J=8.4, 1.8 Hz), 7.58 (2H, m), 4.71 (1H, m), 4.22 (2H, m), 3.40 (1H, dd, J=14.0, 5.3 Hz), 3.22 (1H, dd, J=14.3, 9.6 Hz); HRMS (MALDI) calc for C₁₉H₂₀N₂O₅P (M+H⁺) 387.1110; found 387.1117.

### Example 33

### Phosphoric acid mono-{(R)-3-cyclohexyl-2-[(1-naphthalen-2-yl-methanoyl)-amino]-propyl}ester

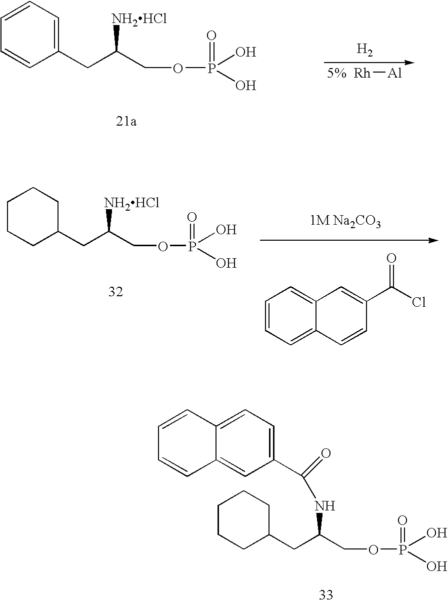

To an aminophosphate 21a (540 mg, 2.02 mmol) solution in acetic acid (70% aqueous solution, 7 mL) was added 5% rhodium on alumina (306 mg). The suspension was kept under hydrogen atmosphere (60 psi) for 15 h. After filtration, the filtrate was concentrated to dryness to give 500 mg of compound 32. A portion of 32 (96 mg, 0.416 mmol) was dissolved in 1 M sodium carbonate solution (2 mL). 2-Naphthoyl chloride (79 mg, 0.416 mmol) was added to the solution. After 15 h, it was acidified to pH˜1 by addition of concentrated HCl solution at 0° C. Preparative HPLC purification afforded 4 mg (2% yield) of the title compound 33. ¹H NMR (CD₃OD): δ 8.40 (1H, s), 8.09-7.87 (4H, m), 7.60 (2H, m), 4.48 (1H, m), 4.06 (2H, m), 1.93 (1H, m); LCMS (ESP): 390 (M−H)⁻.
Aminoalcohol 34:

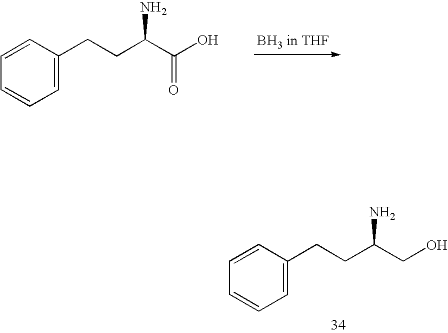

To a THF solution (30 mL) of D-homophenylalanine (5 g, 27.9 mmol) at 0° C. was added borane in THF (1 M, 55.8 mL, 55.8 mmol). After 48 h at 25° C., saturated NaHCO₃ solution (5 mL) was added to the solution and stirred for 4 h. The mixture was concentrated and extracted with methylene chloride (3×50 mL). Combined organic layers were dried (Na₂SO₄) and concentrated. The residue was purified by column chromatography (CH₂Cl₂/MeOH/NH₄OH 951510.5) to give 1.2 g (26% yield) of the compound 34 as a yellowish oil. ¹H NMR (CDCl₃): δ 7.39-7.13 (5H, m), 4.01 (1H, dd, J=11.4, 3.6 Hz), 3.69 (1H, dd, J=11.7, 6.3 Hz), 2.94 (1H, m), 2.71 (2H, t, J=7.8 Hz), 2.09 (1H, m), 1.89 (1H, m); LCMS (ESP): 166 (M+H⁺).
Alcohol 35:

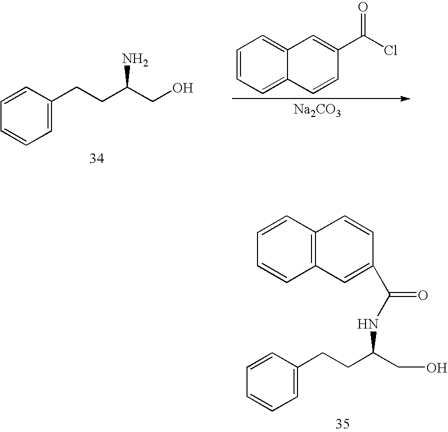

To a sodium carbonate solution (1 M, 2 mL) was added acetonitrile (2 mL), the aminoalcohol 34 (300 mg, 1.82 mmol) and 2-naphthoyl chloride (347 mg, 1.82 mmol). After 10 h, the mixture was extracted with methylene chloride (3×20 mL). The combined organic layers were dried (MgSO₄) and concentrated. The residue was purified by column chromatography (50% EtOAc in hexanes) to afford 220 mg (38% yield) of the compound 35. ¹H NMR (CDCl₃): δ 8.16 (1H, s), 7.93-7.82 (3H, m), 7.74 (1H, dd, J=8.1, 1.8 Hz), 7.55 (2H, m), 7.34-7.15 (5H, m), 6.40 (1H, d, J=7.2 Hz), 4.29 (1H, m), 3.87 (1H, dd, J=11.1, 3.9 Hz), 3.78 (1H, dd, J=10.8, 5.1 Hz), 2.81 (2H, t, J=7.5 Hz), 2.11-2.0 (2H, m); LCMS (ESP): 342 (M+Na⁺); 318 (M−H)⁻; Elemental Analysis for (C₂₁H₂₁NO₂) calc: C, 78.97; H, 6.63; N, 4.39; found: C, 79.07; H, 6.62; N, 4.20.
Phosphate Benzyl Ester 36:

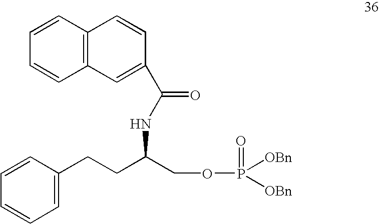

To an acetonitrile solution (6 mL) of the alcohol 35 (206 mg, 0.646 mmol) and 1H-tetrazole (122 mg, 1.74 mmol) was added dibenzyl N,N-diisopropylphosphoramidite (0.433 mL, 1.29 mmol) at 25° C. After 5 h, MCPBA (0.3 g, 77% pure, 2.26 mmol) was added to the suspension. The solution was diluted with methylene chloride (35 mL), washed with concentrated NaHSO₃ solution (2×25 mL), dried over MgSO₄ and concentrated in vacuo. The residue was purified by column chromatography (30% EtOAc in hexanes) to give 0.311 g of the compound 36 in 83% yield. ¹H NMR (CDCl₃): δ 8.28 (1H, s), 7.95-7.78 (4H, m), 7.55 (2H, m), 7.37-7.13 (16H, m), 6.92 (1H, d, J=8.1 Hz), 5.11-4.93 (4H, m), 4.41 (1H, m), 4.21-4.07 (1H, m), 2.72 (2H, t, J=7.8 Hz), 1.97 (2H, m).

### Example 37

### Phosphoric acid mono-{2-[(1-naphthalen-2-yl-methanoyl)-amino]-4-phenyl-butyl}ester

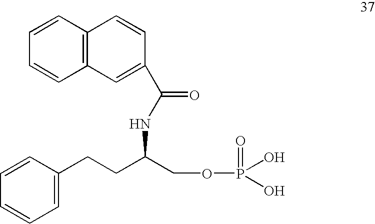

To an ethanol solution of the phosphate benzyl ester 36 (0.305 g, 0.527 mmol) was added 10% palladium on carbon (60 mg). The suspension was kept under hydrogen atmosphere (1 atm) for 15 h and was then filtered through a pad of celite. All solvent was removed in vacuo to give 210 mg (100%) of the title compound 37. ¹H NMR (CD₃OD): δ 8.39 (1H, s), 8.1-7.9 (4H, m), 7.59 (2H, m), 7.3-7.18 (5H, m), 4.37 (1H, m), 4.12 (2H, m), 2.79 (2H, m), 2.06 (2H, m); HRMS (MALDI) calc for C₂₁H₂₃NO₅P (M+H⁺) 400.1314; found 400.1312.
Carboxylic Acid 38:

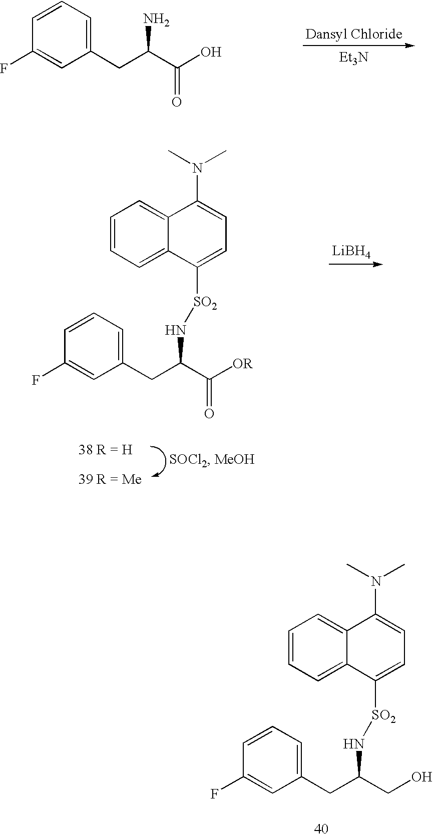

To a methylene chloride solution (8.5 mL) of D-3-fluorophenylalanine (0.5 g, 2.73 mmol) was added triethylamine (1 mL) and dansyl chloride (0.737 g, 2.73 mmol). After 12 h, the mixture was concentrated in vacuo. The residue was dissolved in EtOAc (10 mL) and added to a 5% HCl solution (10 mL). The resulting precipitate 38 was collected by filtration. The solid 38 was dissolved in MeOH (15 mL). After cooling the solution to −20° C., thionyl chloride (1.5 mL) was added. After 15 h at 25° C., the solution was concentrated in vacuo and the residue was dissolved in EtOAc (50 mL). The EtOAc solution was washed with ice-cold sat'd sodium carbonate solution (1×50 mL), dried over MgSO₄ and concentrated to dryness affording 506 mg of the methyl ester 39. The solid 39 was dissolved in THF (10 mL) and added LiBH₄ (76 mg, 3.5 mmol). After 15 h, the reaction was quenched by slow addition of sat'd NH₄Cl solution (1.5 mL). The mixture was extracted by EtOAc (3×25 mL), washed with brine (1×20 mL), dried (MgSO₄) and concentrated. The residue was purified by column chromatography (50% EtOAc in hexanes) to give 308 mg (28% yield for 3 steps) of the desired compound 40. ¹H NMR (CDCl₃): δ 8.43 (1H, d, J=8.4 Hz), 8.14 (1H, dd, J=7.8, 0.9 Hz), 8.04 (1H, d, J=8.7 Hz), 7.40 (2H, m), 7.07 (1H, d, J=7.5 Hz), 6.78 (1H, m), 6.55-6.46 (2H, m), 6.38 (1H, dt, J=9, 1.2 Hz), 5.25 (1H, d, J=10.5 Hz), 3.56 (1H, dd, J=10.8, 3.9 Hz), 3.47-3.31 (3H, m), 2.81 (6H, s), 2.61 (1H, dd, J=13.8, 6.6 Hz), 2.49 (1H, dd, J=13.8, 7.8 Hz).
Phosphate Benzyl Ester 41

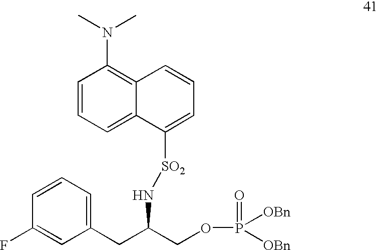

To an acetonitrile solution (1 mL) of the alcohol 35 (53 mg, 0.132 mmol) and 1H-tetrazole (28 mg, 0.394 mmol) was added dibenzyl N,N-diisopropylphosphoramidite (91.2 mg, 0.264 mmol) at 25° C. After 4 h, 50% hydrogen peroxide (1 mL) was added to the suspension at 0° C. The solution was diluted with methylene chloride (25 mL), washed with concentrated NaHSO₃ solution (2×25 mL), dried over MgSO₄ and concentrated in vacuo. The residue was purified by column chromatography (35% EtOAc in hexanes) to give 0.06 g of the compound 41 in 69% yield. ¹H NMR (CDCl₃): δ 8.48 (1H, d, J=8.4 Hz), 8.15 (1H, dd, J=7.5, 0.9 Hz), 8.11 (1H, d, J=8.7 Hz), 7.45 (2H, m), 7.38-7.30 (10H, m), 7.12 (1H, d, J=7.5 Hz), 6.83 (1H, m), 6.59 (1H, td, J=8.7, 2.7 Hz), 6.51 (1H, d, J=7.5 Hz), 6.42 (1H, dt, J=9.6, 1.8 Hz), 5.42 (1H, d, J=8.1 Hz), 5.08-4.94 (4H, m), 3.95-3.84 (2H, m), 3.50 (1H, m), 2.86 (6H, s), 2.59 (1H, dd, J=13.8, 7.2 Hz), 2.46 (1H, dd, J=13.8, 7.2 Hz).

### Example 42

### Phosphoric acid mono-[(R)-2-(5-dimethylamino-naphthalene-1-sulfonylamino)-3-(3-fluoro-phenyl)-propyl]ester

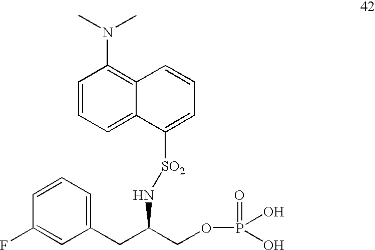

To an ethanol solution of the phosphate benzyl ester 41 (0.06 g, 0.0906 mmol) was added 10% palladium on carbon (10 mg). The suspension was kept under hydrogen atmosphere (1 atm) for 15 h and was then filtered through a pad of celite. The filtrate was concentrated in vacuo. The residue was purified by HPLC to give 20 mg (46% yield) of the title compound 42. ¹H NMR (CD₃OD): δ 8.46 (1H, d, J=8.7 Hz), 8.39 (1H, d, J=9 Hz), 8.20 (1H, d, J=7.5 Hz), 7.77 (1H, d, J=7.5 Hz), 7.72-7.59 (2H, m), 6.69 (1H, m), 6.59 (1H, d, J=8.1 Hz), 6.48-6.34 (2H, m), 4.14 (1H, m), 3.97 (1H, m), 3.62 (1H, m), 3.32 (6H, s), 2.90 (1H, dd, J=14.4, 3.9 Hz), 2.49 (1H, dd, J=10.2, 14.1 Hz); LCMS (ESP): 483 (M+H⁺).
Alcohol 43:

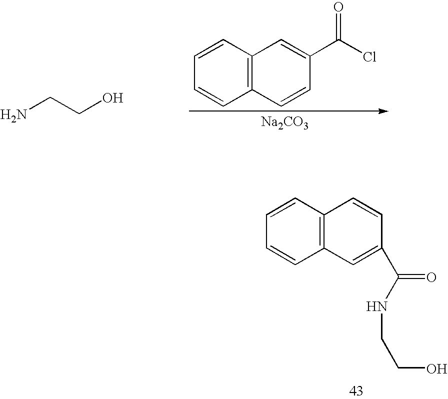

To a sodium carbonate solution (1 M, 5 mL) was added acetonitrile (5 mL), ethanolamine (0.173 mL, 3.12 mmol) and 2-naphthoyl chloride (594 mg, 3.12 mmol). After 15 h, the mixture was extracted with methylene chloride (3×50 mL). The combined organic layers were dried (MgSO₄) and concentrated. The residue was purified by column chromatography (EtOAc) to afford 386 mg (58% yield) of the compound 43. ¹H NMR (CDCl₃): δ 8.31 (1H, s), 7.96-7.78 (4H, m), 7.55 (2H, m), 6.76 (1H, br s), 3.89 (2H, t, J=5.7 Hz), 3.70 (2H, q, J=5.4 Hz); LCMS (ESP): 216 (M+H⁺), 238 (M+Na⁺); Elemental Analysis for (C₁₃H₁₃NO₂) calc: C, 72.54; H, 6.09; N, 6.51; found: C, 72.68; H, 6.09; N, 6.51.
Phosphate Benzyl Ester 44:

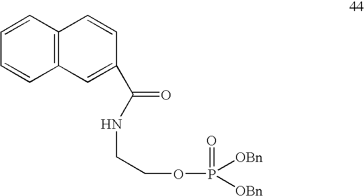

To an acetonitrile solution (10 mL) of the alcohol 43 (321 mg, 1.49 mmol) and 1H-tetrazole (282 mg, 4.02 mmol) was added dibenzyl N,N-diisopropylphosphoramidite (1 mL, 2.99 mmol) at 25° C. After 3 h, MCPBA (77% pure, 900 mg, 5.22 mmol) was added to the suspension at 0° C. The solution was diluted with methylene chloride (75 mL), washed with concentrated NaHSO₃ solution (2×50 mL), dried over MgSO₄ and concentrated in vacuo. The residue was purified by column chromatography (50% to 75% EtOAc in hexanes) to give 0.699 g of the compound 44 in 99% yield. ¹H NMR (CDCl₃): δ 8.34 (1H, s), 7.96-7.81 (4H, m), 7/55 (2H, m), 7.37-7.15 (11H, m), 5.04 (4H, m), 4.19 (2H, m), 3.73 (2H, q, J=5.1 Hz); LCMS (ESP): 498 (M+Na⁺).
Phosphoric Acid 45

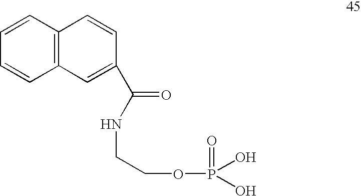

To an ethanol solution of the phosphate benzyl ester 44 (0.672 g, 1.41 mmol) was added 10% palladium on carbon (134 mg). The suspension was kept under hydrogen atmosphere (1 atm) for 2.5 h and was then filtered through a pad of celite. The filtrate was concentrated to dryness in vacuo to give 415 mg (100% yield) of the compound 45 as a white solid. ¹H NMR (CD₃OD): δ 8.42 (1H, s), 8.03-7.87 (4H, m), 7.59 (2H, m), 4.20 (2H, q, J=5.7 Hz), 3.73 (2H, t, J=5.4 Hz); LCMS (ESP): 296 (M+H⁺), 318 (M+Na⁺); 294 (M−H)³¹ ; 589 (2M−H)⁻; Elemental Analysis for (C₁₃H₁₄NO₅P 0.2H₂O) calc: C, 52.25; H, 4.86; N, 4.69; found: C, 52.21; H, 4.95; N, 4.60.
Alcohol 47:

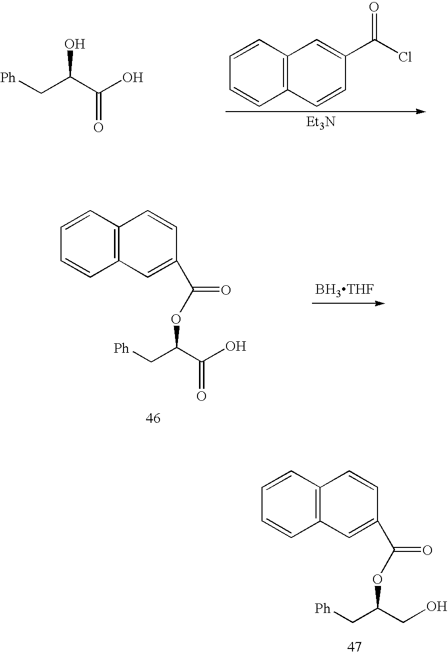

To a methylene chloride solution (5 mL) of D-phenyllactic acid (0.5 g, 3 mmol) at 0° C. was added triethylamine (1 mL) and 2-naphthoyl chloride (0.629 g, 3.3 mmol). After 12 h, the mixture was diluted with EtOAc (80 mL), washed with 5% ice-cold HCl solution (1×SO mL), dried and concentrated. The residue 46 (0.9 g) was dissolved in THF (10 mL). The solution was cooled to −20° C. and 1 M borane in THF solution (3 mL, 3 mmol) was added. After stirring the mixture at 25° C. for 6.5 h, MeOH (15 mL) was added. After 15 min, all of the solvent was removed in vacuo. The residue was dissolved in MeOH (15 mL) and stirred for 1 h. The solution was diluted with EtOAc (100 mL), washed with ice-cold 1M Na₂CO₃ solution (2×50 mL) and brine (1×50 mL), dried and concentrated. The residue was purified by column chromatography (35% EtOAc in hexanes) to give 300 mg (33% yield) of the compound 47. ¹H NMR (CDCl₃): δ 8.57 (1H, s), 8.02 (1H, dd, J=8.7, 1.8 Hz), 7.94 (1H, br d, J=7.8 Hz), 7.90-7.79 (2H, m), 7.57 (2H, m), 7.35-7.18 (5H, m), 5.41 (1H, m), 3.90 (1H, dd, J=12.3, 3.3 Hz), 3.80 (1H, dd, J=12.3, 3.6 Hz), 3.12 (2H, m); LCMS (ESP): 329 (M+Na⁺); 305 (M−H)⁻.
Phosphate Benzyl Ester 48:

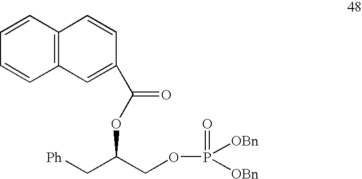

To an acetonitrile solution (10 mL) of the alcohol 43 (60 mg, 0.196 mmol) and 1H-tetrazole (70 mg, 1 mmol) was added dibenzyl N,N-diisopropylphosphoramidite (260 mg, 0.75 mmol) at 25° C. After 3 h, MCPBA (77% pure, 570 mg, 3 mmol) was added to the suspension at 0° C. The solution was diluted with methylene chloride (30 mL), washed with concentrated NaHSO₃ solution (2×30 mL), dried over MgSO₄ and concentrated in vacuo. The residue was purified by column chromatography (1% THF in methylene chloride) to give 80 mg of the compound 48 in 73% yield. ¹H NMR (CDCl₃): δ 8.57 (1H, s), 8.02 (1H, dd, J=8.4, 1.2 Hz), 7.93-7.77 (3H, m), 7.63-7.46 (2H, m), 7.40-7.17 (15H, m), 5.48 (1H, m), 5.10-4.93 (4H, m), 4.19 (2H, m), 3.11 (1H, dd, J=13.5, 6.3 Hz), 3.01 (1H, dd, J=13.5, 7.2 Hz).

### Example 49

### Naphthalene-2-carboxylic acid (R)-2-phenyl-1-phosphonooxymethyl-ethyl ester

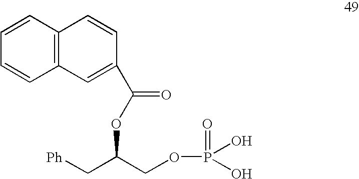

To an ethanol solution of the phosphate benzyl ester 48 (80 mg, 0.141 mmol) was added 10% palladium on carbon (25 mg). The suspension was kept under hydrogen atmosphere (1 atm) for 15 h and was then filtered through a pad of celite. The filtrate was concentrated in vacuo. The residue was purified by HPLC to give 30 mg (55% yield) of the title compound 49. ¹H NMR (CD₃OD): δ 8.53 (1H, s), 7.99-7.80 (4H, m), 7.53 (2H, p, J=6.9 Hz), 7.31-7.08 (5H, m), 5.44 (1H, m), 4.24-4.05 (2H, m), 3.08 (2H, d, J=6.9 Hz); LCMS (ESP): 385 (M−H)⁻; HRMS (MALDI) calc for C₂₀H₂₀O₆P (M+H⁺) 387.0998; found 387.1016.
α-Hydroxycarboxylic Acid:

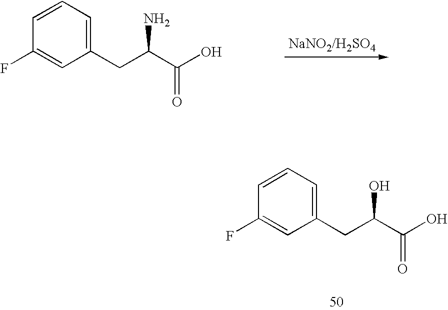

D-3-Fluorophenylalanine (7.98 g, 43.8 mmol) was dissolved in 1 M sulfuric acid solution (140 mL). To the solution at 0° C. was slowly added 6 M NaNO₂ solution (36 mL, 216 mmol) and 3.2 M sulfuric acid (36 mL). The mixture was stirred at 0° C. for 3 h and then at 25° C. for 0.5 h. The solution was extracted with EtOAc (7×75 mL). The combined organic layers were dried and concentrated. Recrystalization from EtOAc/hexanes afforded 5.36 g (67% yield) of the compound α-hydroxycarboxylic acid. ¹H NMR (CDCl₃): δ 7.29 (1H, m), 7.13-7.0 (2H, m), 6.95 (1H, td, J=8.4, 2.4 Hz), 4.36 (1H, dd, J=7.8, 4.2 Hz), 3.12 (1H, dd, J=14.1, 4.5 Hz), 2.93 (1H, dd, J=13.8, 7.8 Hz); LCMS (ESP): 183 (M−H)⁻.
Diol 51:

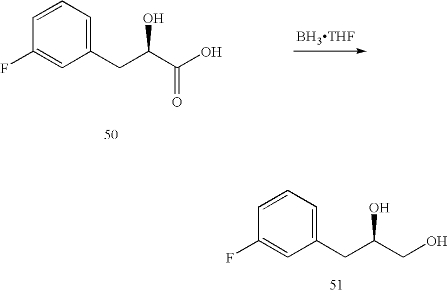

To a THF solution (15 mL) of α-hydroxycarboxylic acid 50 (2.04 g, 11.1 mmol) was added 1 M borane in THF solution (16.6 mL, 16.6 mmol). The mixture was stirred at 25° C. for 16 h. The reaction was quenched by addition of MeOH (15 mL). After 1 h, all of the solvent was removed in vacuo. The residue was dissolved in methylene chloride (15 mL). Saturated NaHCO₃ solution (15 mL) was added to the solution, which was stirred vigorously for 3 h. The mixture was extracted with methylene chloride (2×50 mL). The combined organic layers were dried over Na₂SO₄ and concentrated. The residue was purified by column chromatography (35% to 50% EtOAc in hexanes) to give 969 mg (51% yield) of the compound 51. ¹H NMR (CDCl₃): δ 7.34-7.22 (1H, m), 7.05-6.89 (3H, m), 3.96 (1H, m), 3.71 (1H, dd, J=11.1, 3.3 Hz), 3.53 (1H, dd, J=11.7, 6.9 Hz), 2.78 (2H, m).
Phosphate Benzyl Ester 52:

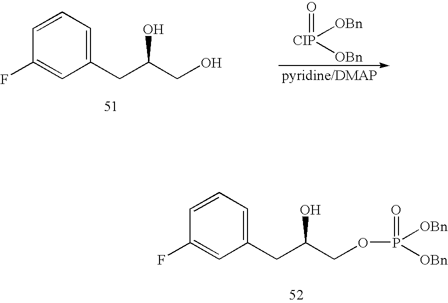

To a methylene chloride solution (4 mL) of the diol 51 (215 mg, 1.26 mmol) was added pyridine (1 mL), 4-dimethylaminopyridine (DMAP) (10 mg) and 10% dibenzyl phosphoryl chloride in benzene (8.8 mL, 2.78 mmol). After 17 h, the solution was diluted with methylene chloride (25 mL), washed with 5% HCl solution (1×30 mL), dried and concentrated. The residue was purified by column chromatography (35% EtOAc in hexanes) to give 68 mg (13% yield) of the compound 52. ¹H NMR (CDCl₃): δ 7.43-7.29 (10H, m), 7.28 (1H, m), 7.00-6.84 (2H, m), 5.15-4.98 (4H, m), 4.03-3.78 (3H, m), 2.71 (2H, m); LCMS (ESP): 431 (M+H⁺), 453 (M+Na⁺).
Ester 53:

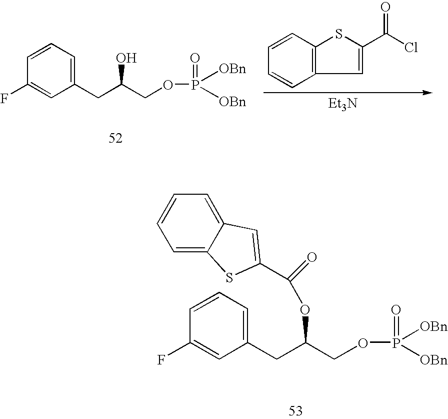

To a methylene chloride solution (2 mL) of the alcohol 52 (67 mg, 0.158 mmol) was added triethylamine (1 mL) and 1-benzothiophene-2-carbonyl chloride (62 mg, 0.316 mmol). After 3 h, the mixture was concentrated in vacuo. The residue was dissolved in methylene chloride (20 mL). The solution was washed with brine (1×20 mL), dried over Na₂SO₄ and concentrated. The residue was purified by column chromatography (5 to 15% EtOAc in hexanes) affording 23 mg (24% yield) of the compound 53. ¹H NMR (CDCl₃): δ 8.01 (1H, s), 7.84 (2H, d, J=8.4 Hz), 5.43 (2H, m), 7.33-7.17 (12H, m), 7.03-6.85 (3H, m), 5.37 (1H, m), 5.02 (4H, m), 4.12 (2H, m), 3.04 (1H, dd, J=13.8, 6.9 Hz), 2.96 (1H, dd, J=14.1, 6.6 Hz).

### Example 54

### Phosphoric acid mono-[(R)-2-[(1-benzo[b]thiophen-2-yl-methanoyl)-amino]-3-(3-fluoro-phenyl)-propyl]ester

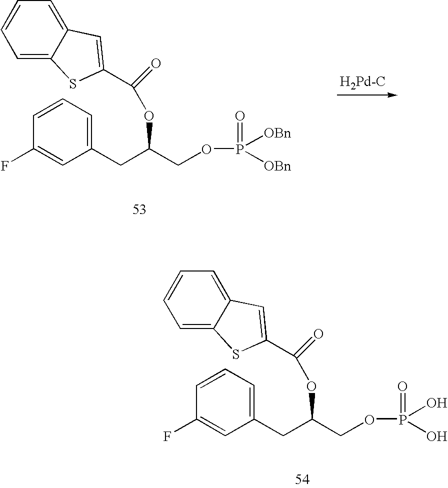

To an ethanol solution of the phosphate benzyl ester 53 (23 mg, 0.038 mmol) was added 10% palladium on carbon (5 mg). The suspension was kept under hydrogen atmosphere (1 atm) for 15 h and was then filtered through a pad of celite. The filtrate was concentrated in vacuo. The residue was purified by HPLC to give 10 mg (64% yield) of the compound 54. ¹H NMR (CD₃OD): δ 8.17 (1H, s), 8.00 (2H, m), 7.53 (2H, m), 7.35 (1H, m), 7.24-7.11 (2H, m), 7.01 (1H, td, J=8.7, 2.7 Hz), 5.53 (1H, m), 4.24 (2H, m), 3.20 (2H, m); HRMS (MALDI) calc for C₁₈H₇₀₆PS (M+H⁺) 411.0468; found 411.0480.
Aminoalcohol 55:

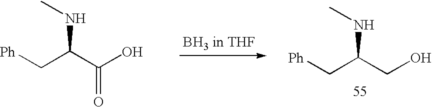

To a THF solution (20 mL) of N-methyl-D-phenylalanine (2.5 g, 14 mmol) was added 1 M borane solution in THF (20.9 mL, 20.9 mmol). After 15 h, sat'd NaHCO₃ solution (5 mL) was added to the solution. The mixture was stirred vigorously for 48 h and then extracted with methylene chloride (3×25 mL). The combined organic layers was dried over MgSO₄ and concentrated. The residue was purified by column chromatography (95/5/0.5 to 90/10/1 CH₂Cl₂/CH₃OH/NH₄OH) to give 710 mg (31% yield) of the compound 55. ¹H NMR (CDCl₃): δ 7.41-7.09 (5H, m), 3.64 (1H, dd, J=10.8, 3.6 Hz), 3.34 (1H, dd, J=10.8, 4.5 Hz), 2.86-2.68 (3H, m), 2.41 (3H, s).
Alcohol 56:

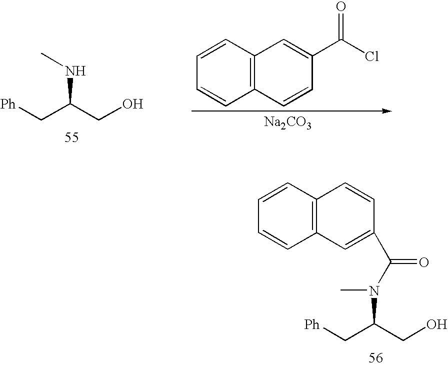

To a sodium carbonate solution (1 M, 4 mL) was added acetonitrile (4 mL), the aminoalcohol 55 (377 mg, 2.28 mmol), and 2-naphthoyl chloride (435 mg, 2.28 mmol). After 15 h, the mixture was extracted with methylene chloride (3×25 mL). The combined organic layers were dried (MgSO₄) and concentrated. The residue was purified by column chromatography (50-100% EtOAc in hexanes) to afford 574 mg (79% yield) of the compound 56. ¹H NMR (CD₃OD): (mixture of two rotamers) δ 3.16 and 2.81 (3H, s); LCMS (ESP): 320 (M+H⁺), 342 (M+Na⁺); Elemental Analysis for (C₂₁H₂₁NO₂) calc: C, 78.97; H, 6.63; N, 4.39; found: C, 79.00; H, 6.76; N, 4.47.
Phosphate Benzyl Ester 57:

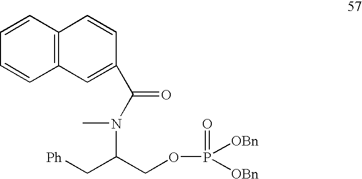

To an acetonitrile solution (8 mL) of the alcohol 56 (322 mg, 1.01 mmol) and 1H-tetrazole (191 mg, 2.72 mmol) was added dibenzyl N,N-diisopropylphosphoramidite (0.678 mL, 2.02 mmol) at 25° C. After 3 h, MCPBA (0.523 g, 77% pure, 3.03 mmol) was added to the suspension. The solution was diluted with methylene chloride (35 mL), washed with concentrated NaHSO₃ solution (2×25 mL), dried over MgSO₄ and concentrated in vacuo. The residue was purified by column chromatography (30 to 50% EtOAc in hexanes) to give 0.23 g of the compound 57 in 40% yield. ¹H NMR (CDCl₃): (mixture of two rotamers) δ 5.06 (4H, m), 3.06 and 2.72 (3H, s); LCMS(ESP): 602 (M+Na⁺).

### Example 58

### Phosphoric acid mono-{[methyl-(1-naphthalen-2-yl-methanoyl)-amino]-phenyl-propyl}ester

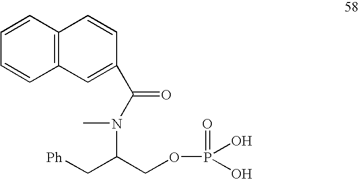

To an ethanol solution of the phosphate benzyl ester 57 (224 mg, 0.387 mmol) was added 10% palladium on carbon (45 mg). The suspension was kept under hydrogen atmosphere (1 atm) for 3 h and was then filtered through a pad of celite. The filtrate was concentrated to dryness in vacuo, affording 155 mg (100% yield) of the title compound 58. ¹H NMR (CD₃OD): (mixture of two rotamers) δ 3.18 and 2.87 (3H, s); LCMS (ESP): 398 (M−H)⁻.

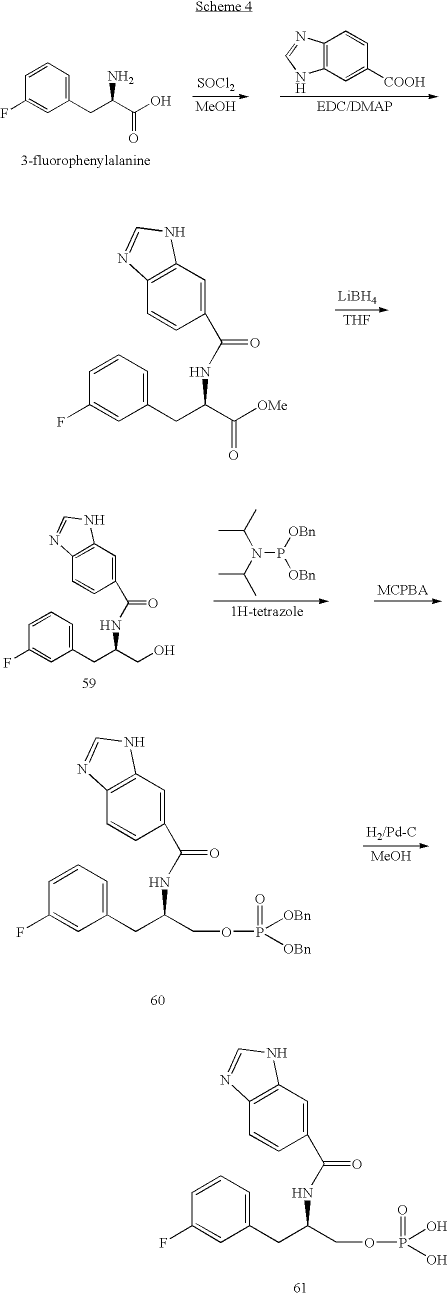

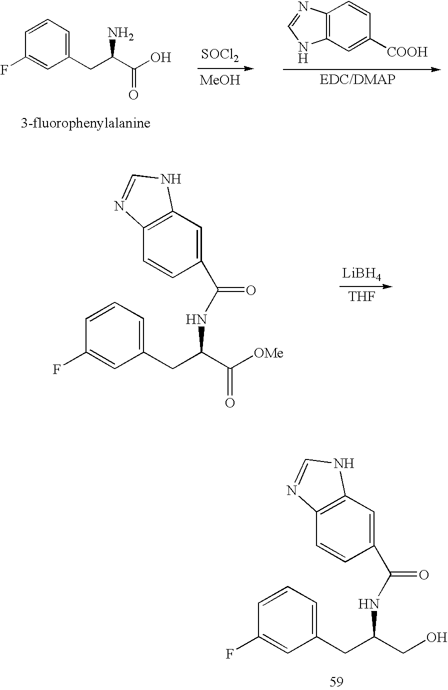

To a 3-fluorophenylalanine (2.2 g, 12 mmol) solution in methanol (12 mL) at −30° C. was added thionyl chloride (1 mL). After stirring at 25° C. for 15 h, the reaction mixture was concentrated in vacuo to give 2.2 g of a solid, which was then dissolved in DMF (25 mL). To the solution was added benzimidazole-6-carboxylic acid (2.18 g, 13.44 mmol), EDC (3.22 g, 16.8 mmol), and DMAP (0.237 g, 2.24 mmol). After 15 h, the mixture was diluted with EtOAc (100 mL), washed with ice-cold 5% NaOH solution (1×80 mL) and brine (3×80 mL), dried (MgSO₄) and concentrated. The resulting crude oil (2 g) was dissolved in THF (10 mL) and the solution was slowly added to LiBH₄ (0.52 g, 24 mmol). After 15 h at 25° C., a solution of NH₄Cl (1 mL) was added slowly to quench the reaction. The suspension was extracted with EtOAc (3×20 mL). The combined organic layers were washed with brine (2×40 mL), dried (MgSO₄) and concentrated. The residue was purified by column chromatography (5% MeOH in CH₂Cl₂), providing 400 mg (11% yield) of the compound 59. LCMS: 314 (M+H⁺, ESP Positive); 312 (M−H⁻, ESP negative).
Phosphate Benzyl Ester 60:

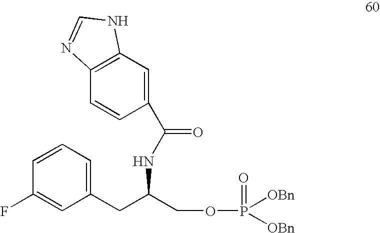

To an acetonitrile solution (6 mL) of the alcohol 59 (188 mg, 0.601 mmol) and 1H-tetrazole (84 mg, 1.20 mmol) was added dibenzyl N,N-diisopropylphosphoramidite (0.302 mL, 0.901 mmol) at 25° C. After 3 h, MCPBA (404 mg, 77% pure, 1.80 mmol) was added to the suspension. The solution was diluted with EtOAc (20 mL), washed with 5% NaHSO₃ solution (1×20mL), dried over Na₂SO₄ and concentrated in vacuo. The residue was purified by column chromatography (98/2/0.2 CH₂Cl₂/CH₃OH/NH₄OH) and further purified by preparative HPLC to give 132 mg of the compound 60 in 38% yield. ¹H NMR (CDCl₃): δ 8.82 (1H, s), 7.89 (1H, d, J=8.10 Hz), 7.75 (1H, d, J=7.54 Hz), 7.47 (1H, d, J=8.67 Hz), 7.37-7.19 (1H, m), 7.00 (1H, d, J=7.35 Hz), 6.91 (2H, d, J=8.85 Hz), 5.14-5.00 (4H, m), 4.53 (1H, m), 4.23-4.01 (2H, m), 3.08-2.96 (1H, dd, J=13.76, 6.97 Hz), 2.91-2.80 (1H, dd, J=13.94, 8.86 Hz); LCMS: 574 (M+H⁺); 596 (M+Na⁺).

### Example 61

### Phosphoric acid mono-[(R)-2-[(1-3H-benzoimidazol-5-yl-methanoyl)-amino]-3-(3-fluoro-phenyl)-propyl]ester

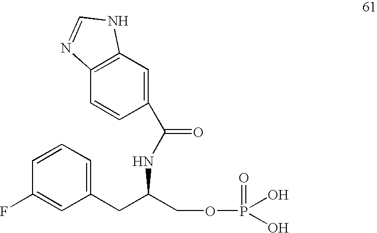

To a methanol solution of the phosphate benzyl ester 60 (132 mg, 0.230 mmol) was added palladium on carbon (10%, 26 mg). The suspension was kept under hydrogen atmosphere (1 atm) overnight, and was then filtered through a pad of celite. Preparative HPLC purification afforded 11 mg (12% yield) of the title compound 61. ¹H NMR (CD₃OD): δ 9.46 (1H, s), 8.26 (1H, s), 8.06-7.86 (1H, dd, J=38.24, 8.85 Hz), 7.30 (1H, m), 7.20-7.04 (2H, m), 6.95 (1H, t, J=10.36 Hz), 4.57 (1H, m), 4.13 (2H, m), 3.18-2.94 (2H, m); LCMS: 394 (M+H⁺); 392 (M−H)⁻; HRMS (MALDI) calc for C₁₇H₁₇FN₃O₅PH (M+H⁺) 394.0962; found 394.0968. 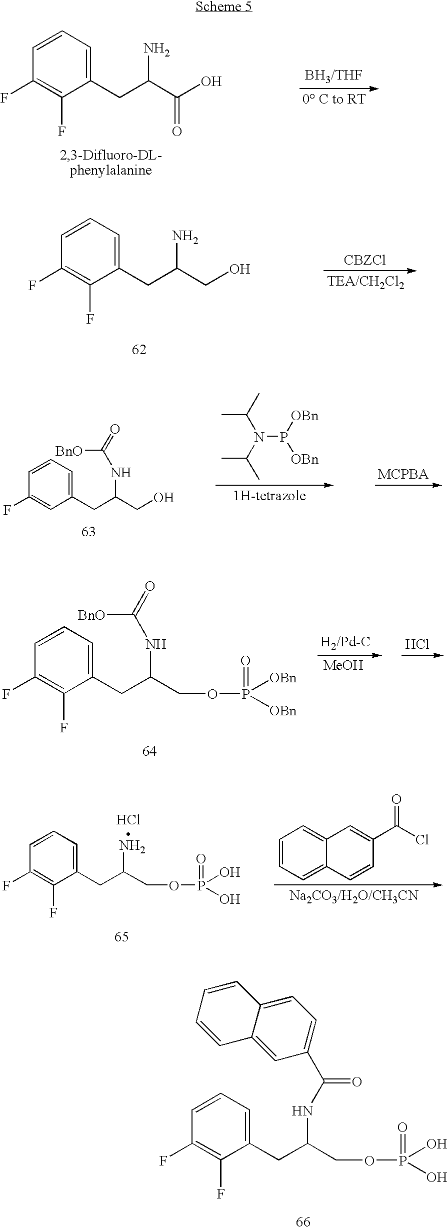 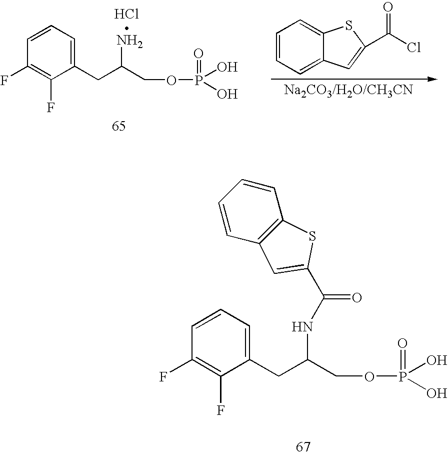
Alcohol 62:

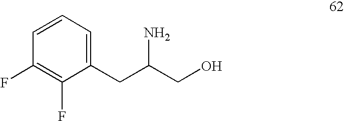

To a tetrahydrofuran solution (25 mL) of 2,3-difluoro-DL-phenylalanine (2.93 g, 14.6 mmol) was slowly added 1 M borane in tetrahydrafuran (36.5 mL, 36.5 mmol) at 0° C. The mixture was warmed to room temperature and stirred overnight. Methanol (20 mL) was added and the solution was stirred vigorously for 1 h. The solvent was evaporated and the procedure was repeated. The 10 residue was dissolved in methylene chloride (50 mL) and stirred vigorously with 1M NaHCO₃ (30 ml) overnight. The mixture was extracted with methylene chloride (3×50 mL). The combined methylene chloride extract was washed with brine (50 mL), dried with Na₂SO₄, and concentrated. After column chromatography purification (95/5/0.5 CH₂Cl₂/CH₃OH/NH₄OH), the compound 62 was obtained in 47% yield (1.28 g). ¹H NMR (CH₃OD): δ 7.20-7.02 (3H, m), 3.54 (1H, dd, J=10.93, 4.52 Hz), 3.44-3.35 (1H, m), 3.14-3.05 (1H, m), 2.93-2.84 (1H, m), 2.75-2.65 (1H, m); LCMS: 18 8.0 (M+H⁺).
Alcohol 63:

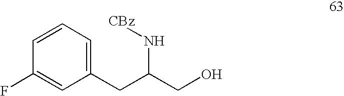

To a methylene chloride solution (30 mL) of 62 (1.28 g, 6.84 mmol) was added triethylamine (1.9 mL, 13.7 mmol) and benzyl chloroformate (1.47 mL, 10.3 mmol). The mixture was stirred overnight and then concentrated. The residue was dissolved in methylene chloride (30 mL) and washed with brine (1×30 mL). The solution was dried (Na₂SO₄) and concentrated. After column chromatography purification (10 to 30% EtOAc in hexane), the compound 63 was obtained in 59% yield (1.30 g). ¹H NMR (CDCl₃): δ 7.27 (5H, m), 7.03-6.86 (3H, m), 5.00 (2H, s), 3.90 (1H, m), 3.70-3.47 (2H, m), 2.88 (1H, d, J=6.80 Hz); MS (ESP): 322.1 (M+H⁺); 344.1 (M+Na⁺).
Phosphate Benzyl Ester 64:

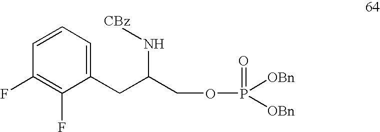

To an acetonitrile solution (20 mL) of the alcohol 63 (1.30 g, 4.05 mmol) and 1H-tetrazole (765 mg, 10.9 mmol) was added dibenzyl N,N-diisopropylphosphoramidite (2.72 mL, 8.10 mmol) at 25° C. After 3 h, MCPBA (3.18 g, 77% pure, 14.2 mmol) was added to the suspension. The solution was diluted with EtOAc (80 mL), washed with 5% NaHSO₃ solution (2×80 mL), dried over MgSO₄ and concentrated in vacuo. The residue was purified by column chromatography (10-30% EtOAc in hexane) to give 2.16 g of the compound 64 in 92% yield. ¹H NMR (CDCl₃): δ 7.51-7.38 (15H, m), 7.18-6.93 (3H, m), 5.18-5.08 (6H, m), 4.21-3.93 (3H, m), 2.95 (2H, d, J=6.42 Hz); LCMS: 604.2 (M+Na⁺).
Amino Phosphate 65:

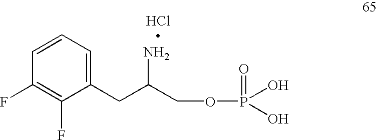

To a methanol solution of the phosphate benzyl ester 64 (2.16 g, 3.72 mmol) was added palladium on carbon (10%, 300 mg). The suspension was kept under hydrogen atmosphere (1 atm) for 4 h, and was then filtered through a pad of celite. The collected solid was washed with methylene chloride. The mixture of the solid and celite was suspended in 5% HCl solution and stirred for 20 min. After filtration, the filtrate was concentrated to dryness, affording 910 mg of the compound 65 in 92% yield. ¹H NMR (DMSO): δ 6.48-6.32 (3H, m), 3.38-3.28 (1H, m), 3.24-3.13 (1H, m), 3.00-2.88 (1H, m), 2.32 (2H, d, J=7.18 Hz); LCMS: 268.0 (M+H⁺); 266.1 (M−H)⁻.

### Example 66

### Phosphoric acid mono-{3-(2,3-difluoro-phenyl)-2-[(1-naphthalen-2-yl-methanoyl)-amino]-propyl}ester

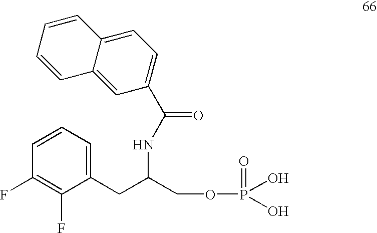

To a sodium carbonate solution (1 M, 5 mL) was added the aminophosphate 65 (226 mg, 0.745 mmol) and 2-naphthyl chloride (142 mg, 0.745 mmol). After 15 h, it was acidified to pH˜1 by the addition of 1 M HCl solution at 0° C. Preparative HPLC purification afforded 135 mg (43% yield) of the title compound 66. ¹H NMR (CD₃OD): δ 8.31 (1H, s), 7.98-7.91 (3H, m), 7.82 (1H, d, J=1.72 Hz), 7.60 (2H, m), 7.20-7.06 (3H, m), 4.82 (1H, m), 4.18 (2H, m), 3.24 (1H, dd, J=13.93, 5.49 Hz), 3.07 (1H, dd, J=13.69, 8.84 Hz); LCMS: 420.2 (M−H)⁻; Elemental Analysis for (C₂₀H₁₈F₂NO₅P) calc: C, 57.01; H, 4.31; N, 3.32; found: C, 56.80; H, 4.55; N, 3.27.

### Example 67

### Phosphoric acid mono-[2-[(1-benzo[b]thiophen-2-yl-methanoyl)-amino]-3-(2,3-difluoro-phenyl)-propyl]ester

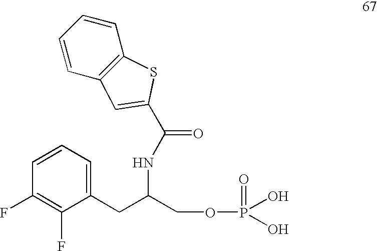

To a sodium carbonate solution (1 M, 5 mL) was added the aminophosphate 65 (199 mg, 0.656 mmol) and benzo[b]thiophene-2-carbonyl chloride (129 mg, 0.656 mmol). After 15 h, it was acidified to pH˜1 by the addition of 1 M HCl solution at 0° C. Preparative HPLC purification afforded 75 mg (27% yield) of the title compound 67. ¹H NMR (CD₃OD): δ 7.73-7.62 (3H, m), 7.23-7.15 (2H, m), 6.95-6.78 (3H, m), 4.42-4.29 (1H, m), 3.97-3.82 (2H, m), 2.98 (1H, dd, J=13.97, 4.91 Hz), 2.80 (1H, dd, J=13.98, 10.20 Hz); LCMS: 426.0 (M−H⁻); Elemental Analysis for (C₁₈H₁₆F₂NO₅PS. 0.20H₂O) calc: C, 50.16; H, 3.84; N, 3.25; found: C, 50.04; H, 3.85; N, 3.32. 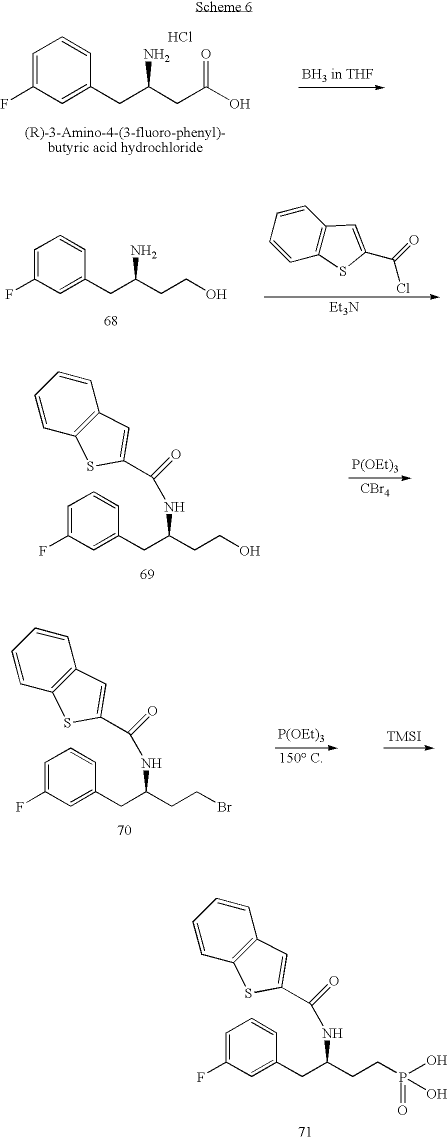
Amine 68:

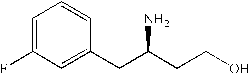

To a stirred solution of lithium borohydride (3 eq) in THF (45 mL) was added chlorotrimethylsilane (10.09 mL, 0.080 mol, 6 eq). The solution was stirred five minutes at room temperature. (R)-3-Amino-4-(3-fluoro-phenyl)-butyric acid hydrochloride (3.10 g, 13.24 mmol) was added portion wise, and the reaction was stirred overnight at room temperature. The reaction was quenched with methanol, and the solvents were removed in vacuo. The residue was diluted with H₂O, and the pH was brought to 12 with aqueous NaOH. The product was extracted with chloroform, and the organic phase was separated, washed sequentially with water and brine, dried (MgSO₄), and the solvent evaporated to give 2.21 g (91%) of a clear oil 68. ¹H NMR (benzene-d₆): δ 1.10-1.30 (m, 2H), 1.20-1.60 (br, 3H), 1.91 (dd, 1H, J=8.3, 13.3 Hz), 2.18 (dd, 1H, J=5.2, 13.3 Hz), 2.54-2.62 (m, 1H), 3.62-3.70 (m, 1H), 3.73-3.79 (m, 1H), 6.48 (d, 1H, J=7.5 Hz), 6.55-6.59 (m, 1H), 6.69-6.76 (m, 1H), 6.83-6.90 (m, 1H); IR (neat) 1588, 1487, 1449, 1251, 1141, 1065, 783 cm⁻¹; R_{f}=0.18 (5% methanolic ammonia/CHCl₃); LCMS 184 (M+H).
Alcohol 69:

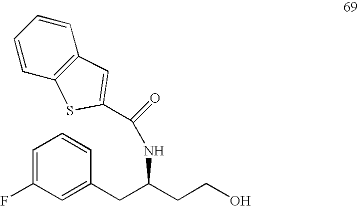

Amine 68 (1.05 g, 5.72 mmol) was dissolved in CH₂Cl₂ (60 mL). Triethylamine (0.88 mL, 6.30 mmol) was added, followed by benzo[b]thiophene-2-carbonyl chloride (1.12 g, 5.72 mmol). The reaction was stirred at room temperature for one hour, and the solvent was removed in vacuo. The product was purified by flash column chromatography eluting with a gradient of 30-60% EtOAc/hexanes (R_{f}=0.24 (50% EtOAc/hexanes) to give 1.87 g (91%) of a white solid 69. ¹H NMR (DMSO-d₆): δ 1.68-1.75 (m, 2H), 2.87 (d, 2H, J=7.0 Hz), 3.43-3.52 (m, 2H), 4.20-4.27 (m, 1H), 4.42 (t, 1H, J=5.1 Hz), 6.94-7.00 (m, 1H), 7.03-7.08 (m, 2H), 7.25-7.32 (m, 1H), 7.39-7.46 (m, 2H), 7.90-8.01 (m, 2H), 8.05 (s, 1H), 8.50 (d, 1H, J=8.5 Hz); LCMS 344 (M+H).
Bromide 70:

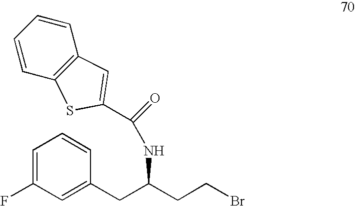

Alcohol 69 (1.77 g, 5.14 mmol) was partially dissolved in CH₂Cl₂ (50 mL) and cooled to 0° C. Triethyl phosphite (1.77 mL, 10.29 mmol) was added, followed by CBr₄ (3.41 g, 10.29 mmol). The ice bath was removed, and the reaction was allowed to warm to room temperature over 5 hours. The reaction was poured into CH₂Cl₂/H₂O. The organic phase was separated, washed with brine, dried (MgSO₄) and evaporated. The crude product was purified by flash column chromatography eluting with a gradient of 3-50% EtOAc/hexanes (R_{f}=0.18 (10% EtOAc/hexanes) to give 0.45 g (21%) of a white solid 70. ¹H NMR (DMSO-d₆): δ 2.05-2.16 (m, 2H), 2.88 (d, 2H, J=6.9 Hz), 3.50-3.59 (m, 2H), 4.25-4.29 (m, 1H), 6.96-7.08 (m, 3H), 7.26-7.33 (m, 1H), 7.40-7.47 (m, 2H), 7.92-8.01 (m, 2H), 8.06 (s, 1H), 8.57 (d, 1H, J=8.5 Hz); LCMS 406, 408 (M+H).

### Example 71

### ([(R)-3-[(Benzo[b]thiophene-2-carbonyl)-amino]4-(3-fluoro-phenyl)-butyl]-phosphonic acid)

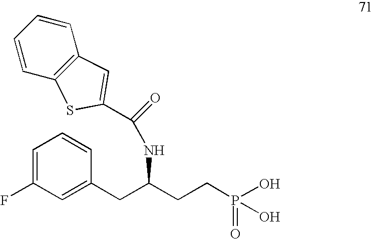

Bromide 70 (0.38 g, 0.93 mmol) was suspended in triethyl phosphite (5 mL) and placed in a microwave apparatus for 15 minutes at 150° C. upon which the reaction goes clear. The solvent was removed in vacuo. The crude product was chromatographed by flash silica gel chromatography eluting with a gradient of 0-2% MeOH/CHCl₃ giving 0.11 g of the diethyl phosphonate as a clear oil (R_{f}=0.24, 3% MeOH/CHCl₃). The oil was dissolved in CH₂Cl₂ (3 mL) and treated with bromotrimethylsilane (0.15 mL, 1.2 mmol). The reaction was stirred overnight at room temperature. The solvent was removed, and the residue triturated with water. The resulting white precipitate was filtered, washed with water, and dried to give 0.07 g (17%) of the title compound as a white solid 71. ¹H NMR (DMSO-d₆): δ 1.49-1.80 (m, 4H), 2.80-2.93 (m, 2H), 4.14-4.19 (m, 1H), 6.94-7.00 (m, 1H), 7.07-7.09 (m, 2H), 7.24-7.32 (m, 1H), 7.40-7.46 (m, 2H), 7.91-8.00 (m, 2H), 8.08 (s, 1H), 8.60 (d, 1H, J=8.5 Hz); HRMS calculated for C₁₉H₂₀NO₄PSF 408.0835 (M+H), found 408.0830; Anal. (C₁₉H₁₉NO₄PSF) C, H, N. 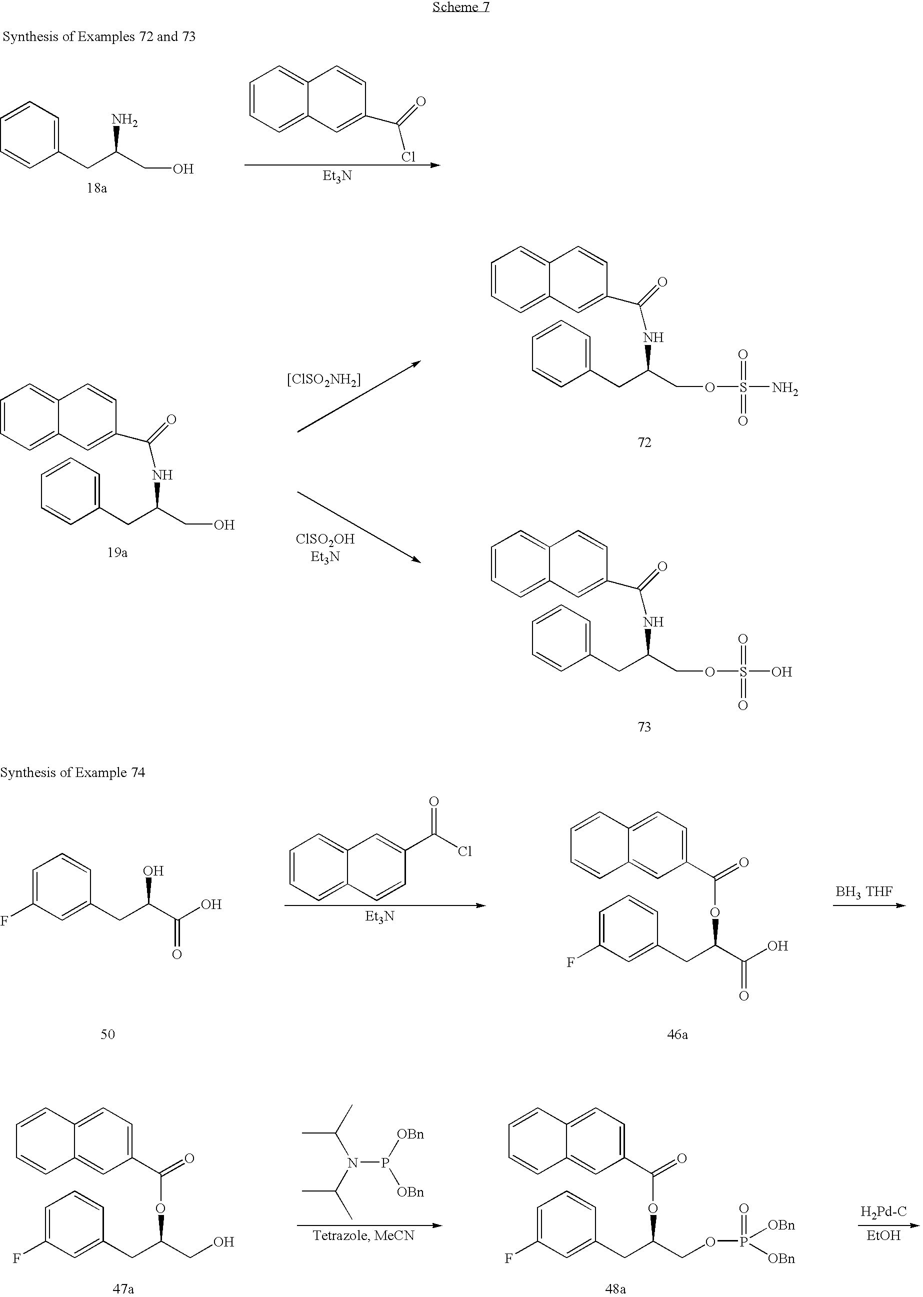 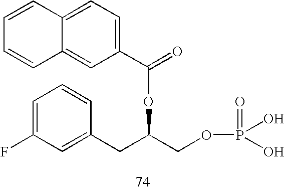
Alcohol 19a:

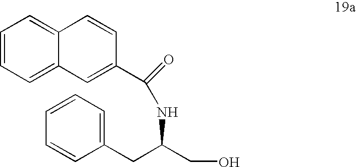

To a CH₂Cl₂ solution (40 mL) of D-phenylalaninol (2.26 g, 14.9 mmol) at 0° C. was added triethylamine (3.11 mL, 22.4 mmol) and 2-naphthoyl chloride (3.13 g, 16.4 mmol). After 15 h at 25° C., the mixture was diluted with CH₂Cl₂ (50 mL), washed with brine (3×50 mL), dried and 10 concentrated. The residue was purified by column chromatography (2% MeOH in CH₂Cl₂) to give 1.33 g (30% yield) of the compound 19a as a white solid. ¹H NMR (CDCl₃): δ 8.16 (1H, s), 7.93-7.83 (3H, m), 7.73 (1H, dd, J=8.5, 1.7 Hz), 7.55 (2H, m), 7.39-7.23 (5H, m), 6.48 (1H, d, J=9 Hz), 4.43 (1H, m), 3.86 (1H, dd, J=11.1, 3.6 Hz), 3.77 (1H, dd, J=11, 4.9 Hz); Elemental Analysis for (C₂₀H₁₉NO₂) calc: C, 78.66; H, 6.27; N, 4.59; found: C, 78.41; H, 6.37; N, 4.52.

### Example 72

### Sulfamic acid 2-[(1-naphthalen-2-yl-methanoyl)-amino]-3-phenyl-propyl ester

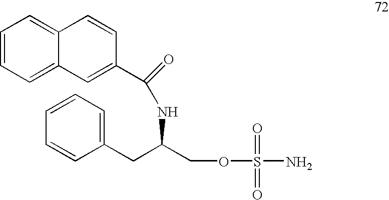

To an acetonitrile (1 mL) solution of chlorosulfonyl isocyanate (136 μL, 1.57 mmol) at 0° C. was added water (28 μL, 1.57 mmol). After stirring for 1.5 h at 0° C., acetonitrile (1 mL), pyridine (149 μL, 1.57 mmol), and the alcohol (255 mg, 0.836 mmol) were added to the solution. The mixture was stirred at 25° C. for 15 h, diluted with EtOAc (20 mL), and washed with ice-cold 2% HCl solution (1×20 mL). Column chromatography (30-50% EtOAc in hexanes) purification gave 19 mg (6% yield) of the title compound 72. ¹H NMR (CD₃OD): δ 8.29 (1H, S), 8.0-7.89 (3H, M), 7.80 (1H, dd, J=8.7, 1.7 Hz), 7.58 (2H, m), 7.4-7.18 (5H, m), 4.66 (1H, m), 4.29 (2H, m), 3.08 (2H, m); LCMS (ESP): 385 (M+H⁺), 407 (M+Na⁺).

### Example 73

### Sulfuric acid mono-{2-[(1-naphthalen-2-yl-methanoyl)-amino]-3-phenyl-propyl}ester

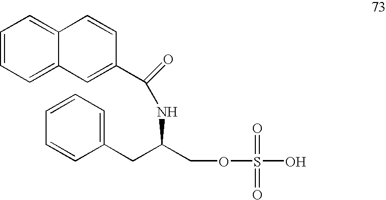

To a CH₂Cl₂ (5 mL) solution of the alcohol (105 mg, 0.344 mmol) at −30° C. was added triethylamine (0.5 mL) and chlorosulfonic acid (116 mg, 90 μl, 1 mmol). After stirring at 25° C. for 15 h, the mixture was diluted with EtOAc (10 mL), washed with ice-cold 2% HCl solution (1×15 mL), dried and concentrated. The residue was purified by column chromatography to afford 131 mg (87% yield) of the title compound 73 as a white solid. ¹H NMR (CD₃OD): δ 8.34 (1H, s), 7.91 (4H, m), 7.58 (2H, m), 7.4-7.15 (5H, m), 4.59 (1H, m), 4.23 (1H, dd, J=10.5, 4.3 Hz), 4.13 (1H, dd, J=10.6, 5.5 Hz), 3.07 (2H, m); HRMS (MALDI) calc for C₂₀H₁₈NO₅SNa₂ (M−H⁺+2Na⁺) 430.0695; found 430.0676.
Alcohol 47a:

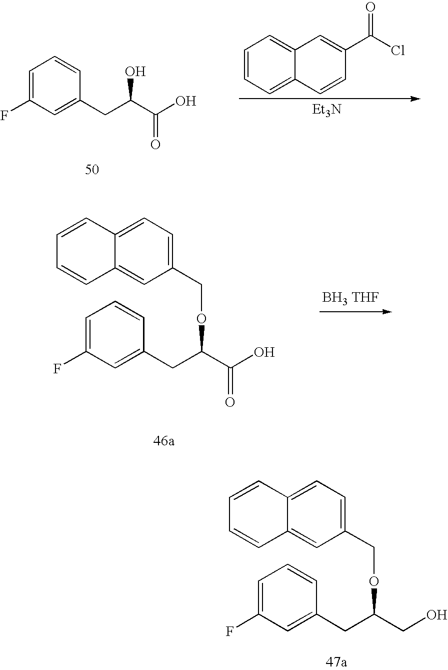

Alcohol 47a was prepared as described in the synthesis of compound 47. In the first step (preparation of 46a), hydroxyl carboxylic acid (760 mg, 4.13 mmol), 2-naphthoyl chloride (866 mg, 4.54 mmol), and triethylamine (2.9 mL) were used. In the second step, 1 M borane in THF (4.73 mL) was used. After column chromatography (40% EtOAc in hexanes) purification, the compound 47a was obtained as a crude oil (137 mg). LCMS: 325 (M+H⁺).
Benzyl Ester 48a:

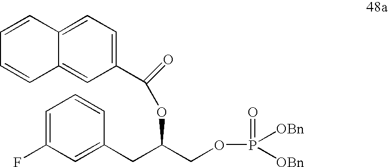

Benzyl Ester 48a was prepared as described in the synthesis of 48 using the alcohol 47a (137 mg, 0.423 mmol), 1H-tetrazole (68 mg, 0.973 mmol), dibenzyl N,N-diisopropylphosphoramidite (0.213 mL, 0.634 mmol), and MCPBA (380 mg, 77% pure, 1.69 mmol). After column chromatography purification (10% to 20% EtOAc in hexanes), the compound 48a was obtained as crude oil (330 mg), which was carried forward to the next step.

### Example 74

### Naphthalene-2-carboxylic acid (R)-1-(3-fluoro-benzyl)-2-phosphonooxy-ethyl ester

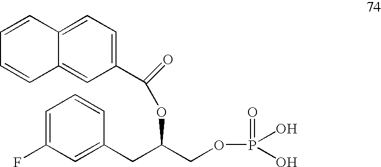

Example 74 was prepared as described in the synthesis of 49 using the crude benzyl ester (330 mg) and 10% palladium on carbon (70 mg). Preparative HPLC purification gave 70 mg of the title compound 74 (31% yield from the alcohol). ¹H NMR (CD₃OD): δ 8.56 (1H, s), 8.02-7.8 (4H, m), 7.53 (2H, m), 7.23 (1H, m, 7.06 (2H, m), 6.89 (1H, m), 4.40 (1H, dd, J=12, 3 Hz), 4.26 (1H, dd, J=11.8, 6 Hz), 3.12 (2H, m); LCMS (ESP Negative): 403 (M−H). HRMS (MALDI): calc for C₂₀H₉O₆FP (M+H⁺) 405.0903; found 405.0902. 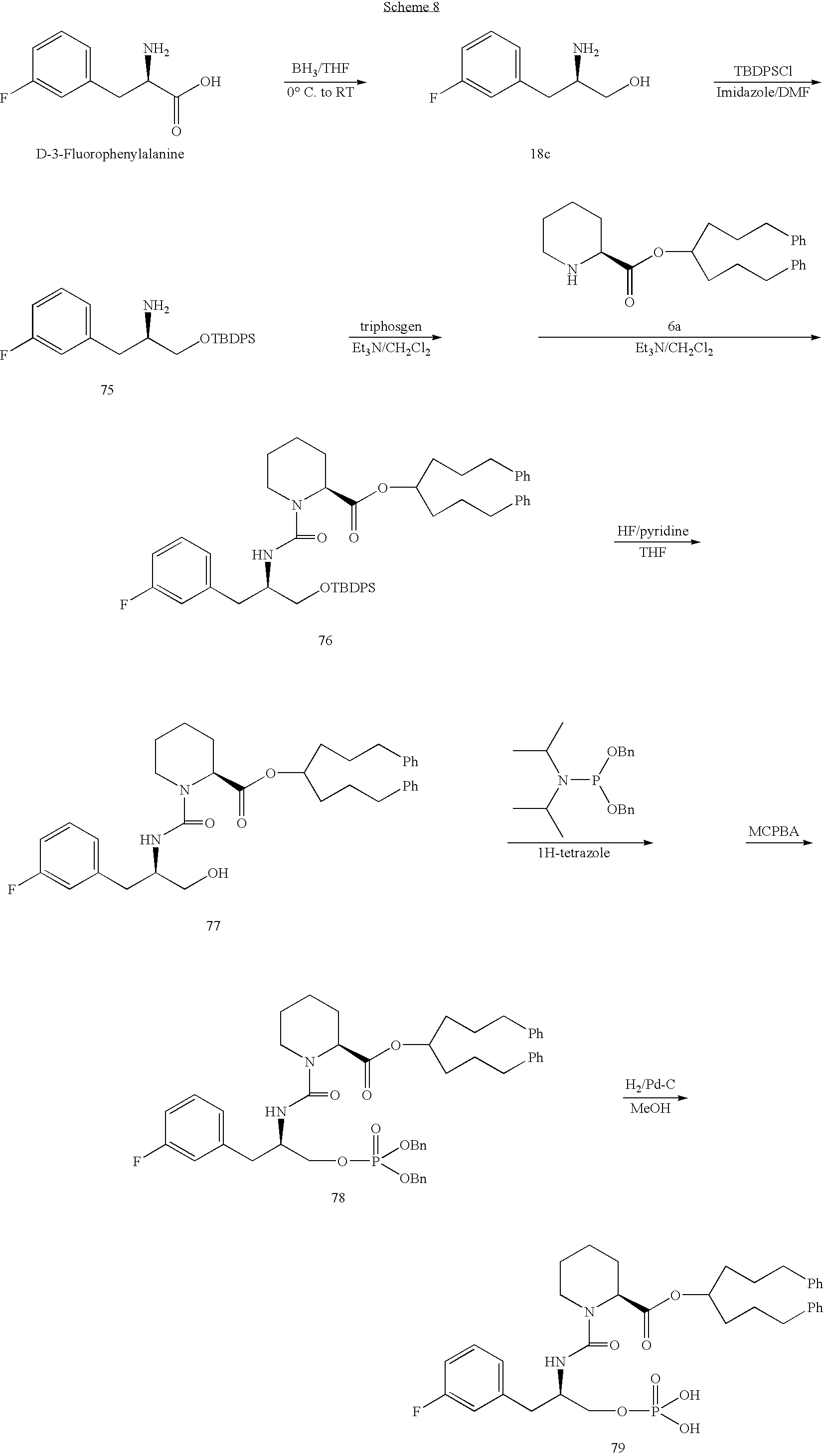
D-Phenylalaninol 18c:

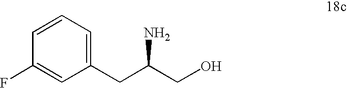

To a tetrahydrofuran solution (30 mL) of D-3-Fluorophenylalanine (5.00 g, 27.3 mmol) was slowly added 1 M borane in tetrahydrofuran (68.3 mL, 68.3 mmol) at 0° C. The mixture was warmed to room temperature and stirred overnight. Methanol (40 mL) was added and stirred vigorously for 1 h. The solvent was evaporated and the procedure was repeated. The residue was dissolved in methylene chloride (100 mL) and stirred vigorously with 1 M NaHCO₃ (50 ml) overnight. The mixture was extracted with methylene chloride (3×50 mL). The combined methylene chloride extract was washed with brine (50 mL), dried with Na₂SO₄, and concentrated. After column chromatography purification (95/5/0.5 CH₂Cl₂/CH₃OH/NH₄OH), the compound 18c was obtained in 28% yield (1.29 g). ¹H NMR (CD₃OD): δ 7.34 (1H, m), 7.09-6.92 (3H, m), 3.54 (1H, dd, J=10.74, 4.33 Hz), 3.39 (1H, dd, J=10.74, 6.60 Hz), 3.13-3.02 (1H, m), 2.83 (1H, dd, J=13.37, 6.21 Hz), 2.62 (1H, dd, J=13.38, 7.73 Hz); LCMS: 170.1 (M+H⁺).
Amine 75:

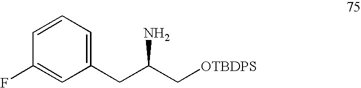

To a DMF solution (10 mL) of D-phenylalaninol 18c (1.30 g, 7.70 mmol) was added imidazole (1.05 g, 15.4 mmol) and t-butyldiphenylchlorosilane (TBDPSCI) (2.40 mL, 9.24 mmol). After stirring overnight, the mixture was diluted with ether (80 mL), washed with saturated ammonium chloride (1×50), brine (1×50 mL) and dried (Na₂SO₄). The solvent was removed in vacuo. The residue was purified by column chromatography (95/5 CH₂Cl₂/CH₃OH) to afford 2.35 g (74% yield) of the compound 75. ¹H NMR (CDCl₃): δ 7.87 (4H, d, J=6.22 Hz), 7.47-7.34 (6H, m), 7.21 (1H, t, J=7.35 Hz), 6.91 (3H, dd, J=16.58, 9.42 Hz), 3.61 (2H, dd, J=9.98, 4.71 Hz), 3.52 (1H, dd, J=9.98, 6.22 Hz), 3.17-3.08 (1H, m), 2.80 (1H, dd, J=13.56, 5.27 Hz), 2.52 (1H, dd, J=13.38, 8.29 Hz), 2.52 (1H, dd, J=13.38, 8.29 Hz), 1.08 (9H, s); LCMS: 408.2 (M+H⁺), 430.2 (M+Na⁺).
Silyl Ether 76:

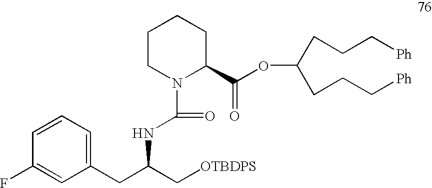

To a methylene chloride solution (15 mL) of the amine 75 (2.35 g, 5.77 mmol) and triethylamine (1.53 mL, 11.0 mmol) was added a methylene chloride solution (2 mL) of triphosgene (1.03 g, 3.46 mmol). After 2 h, the solution was heated at reflux for 1.5 h, and was then cooled to 25° C. A methylene chloride solution (25 mL) of the amine 6a (2.19 g, 5.77 mmol) was added. After 15 h, the reaction solution was diluted with CH₂Cl₂ (80 mL), washed with brine (2×80 mL), dried (Na₂SO₄) and concentrated in vacuo. The residue was purified by flash column chromatography (5-20% EtOAc in hexane) to afford 3.92 g (79% yield) of the compound 76. ¹H NMR (CDCl₃): δ 7.65-7.58 (4H, m), 7.47-7.33 (6H, m), 7.25-7.18 (4H, m), 7.17-7.07 (7H, m), 6.88-6.82 (3H, m), 5.06 (1H, d, J=8.29 Hz), 5.01-4.94 (1H, m), 4.93-4.88 (1H, m), 4.11-3.97 (1H, m), 3.55 (2H, d, J=3.02 Hz), 3.42-3.30 (1H, m), 3.15-2.81 (3H, m), 2.63-2.49 (4H, m), 2.23-2.13 (1H, m), 1.72-1.46 (10H, m), 1.34-1.20 (4H, m), 1.10 (9H, s).
Alcohol 77:

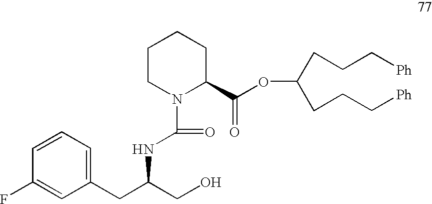

To a THF solution (30 mL) of the silyl ether 76 (3.92 g, 4.80 mmol) was added 1 M HF/pyridine (7 mL) at 0° C. The reaction mixture was warmed to 25° C. after 30 minutes and stirred overnight. The THF was removed by vacuum. The residue was dissolved in methylene chloride (50 mL) and washed with cold 1 M HCl (2×50). The solution was concentrated. The resulting residue was purified by column chromatography (30% EtOAc in hexane) to give 2.26 g (82% yield) of the compound 77. ¹H NMR (CDCl₃): δ 7.33-7.23 (4H, m), 7.22-7.12 (6H, m), 7.06-6.89 (3H, m), 4.99 (1H, m), 4.90 (1H, m), 4.05-3.94 (1H, m), 3.72 (1H, d, J=3.20 Hz), 3.68 (1H, d, J=3.02 Hz), 3.60-3.50 (1H, m), 3.39 (1H, d, J=111.68 Hz), 3.18-3.05 (1H, m), 2.99-2.78 (3H, m), 2.60 (5H, m), 2.21 (1H, d, J=12.81 Hz), 1.81-1.51 (9H, m), 1.50-1.36 (1H, m), 1.33-1.20 (1H, m).
Phosphate Benzyl Ester 78:

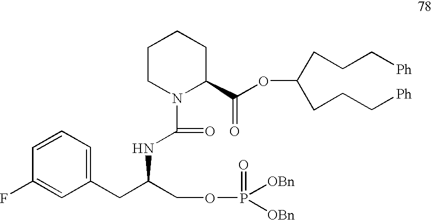

To an acetonitrile solution (35 mL) of the alcohol 77 (2.26 g, 3.94 mmol) and 1H-tetrazole (552 mg, 7.88 mmol) was added dibenzyl N,N-diisopropylphosphoramidite (1.98 mL, 5.91 mmol) at 25° C. After 3 h, MCPBA (2.65 g, 77% pure, 11.8 mmol) was added to the suspension. The solution was diluted with EtOAc (100 mL), washed with concentrated NaHSO₃ solution (2×80 mL), dried over MgSO₄ and concentrated in vacuo. The residue was purified by column chromatography (10-30% EtOAc in hexane) to give 2.23 g of the compound 78 in 68% yield. ¹H NMR (CDCl₃): δ 7.18 (12H, m), 7.09-7.03 (3H, m), 7.03-6.91 (6H, m), 6.81-6.67 (3H, m), 5.57-5.45 (1H, m), 4.95-4.84 (5H, m), 4.84-4.73 (2H, m), 4.00-3.91 (2H, m), 3.81-3.58 (2H, m), 3.43-3.30 (1H, m), 3.00-2.87 (1H, m), 2.83-2.73 (1H, m), 2.54-2.35 (5H, m), 2.08-1.97 (1H, m), 1.55-1.32 (5H, m), 1.13-1.06 (6H, m).

### Example 79

### (S)-1-[(R)-2-(3-Fluoro-phenyl)-1-phosphonooxymethyl-ethylcarbamoy]-piperidine-2-carboxylic acid 4-phenyl-1-(3-phenyl-propyl)-butyl ester

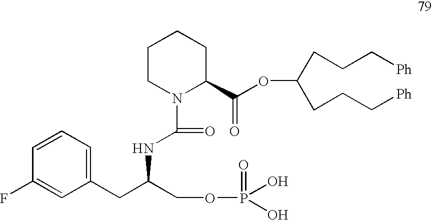

To a methanol solution of the phosphate benzyl ester 78 (400 mg, 0.479 mmol) was added palladium on carbon (10%, 80 mg). The suspension was kept under hydrogen atmosphere (1 atm) overnight, and was then filtered through a pad of celite. The filtrate was purified by preparative HPLC, affording 160 mg of the compound 79 in 51% yield. ¹H NMR (CD₃OD): δ 7.34-7.19 (5H, m), 7.19-7.09 (6H, m), 7.09-6.88 (3H, m), 5.05-4.95 (1H, m), 4.87-4.81 (1H, m), 4.17-4.06 (1H, m), 3.93 (2H, t, J=5.09 Hz), 3.75-3.65 (1H, m), 3.07-2.90 (2H, m), 2.88-2.76 (1H, m), 2.69-2.51 (4H, m), 2.18 (1H, d, J=13.56 Hz), 1.72-1.51 (11H, m), 1.45-1.14 (2H, m); HRMS (MALDI) calc for C₉H₁₅NO₄P (M+H⁺) 655.2954; found 655.2958.

Scheme 9: Synthetic Routes to Prodrugs

1. Synthesis of Acetoxymethyl Phospate Ester:

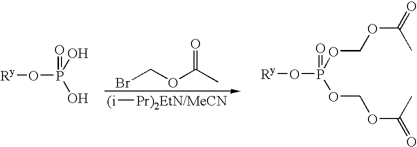

2. Synthesis of Phenyl Phospate Ester:

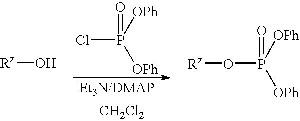

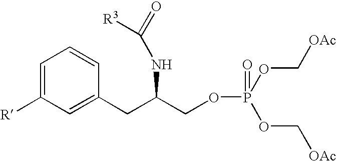

| | | | |
|---|---|---|---|
| 80 | R¹¹ = | H; R³ = | 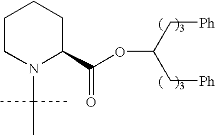 |
| 81 | R¹¹ = | H; R³ = | 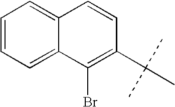 |
| 82 | R¹¹ = | H; R³ = | 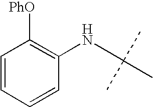 |
| 83 | R¹¹ = | H; R³ = | 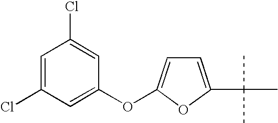 |
| 84 | R¹¹ = | F; R³ = | 2-naphthyl |
| 85 | R¹¹ = | F; R³ = | 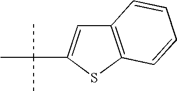 |
| 91 | R¹¹ = | H; R³ = | 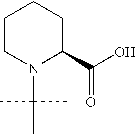 |
| 92 | R¹¹ = | F; R³ = | 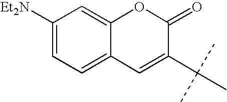 |
| 89 | | | 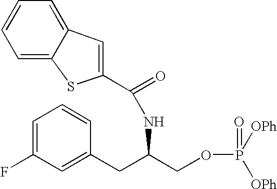 |
| | | | 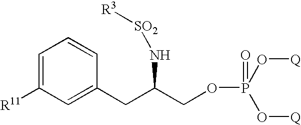 |
| 86 | R¹¹ = | F; Q = Ph; R³ = | 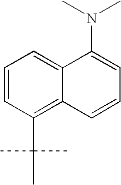 |
| 90 | R¹¹ = | H; Q = —CH₂OAc; R³ = | 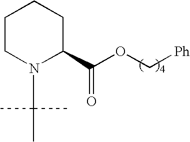 |
| 93 | | | 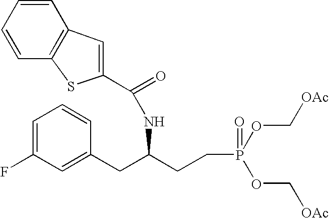 |

### Example 80

### 1-[1-(Bis-acetoxymethoxy-phosphoryloxymethyl)-2-phenyl-ethylcarbamoyl]-piperidine-2S-carboxylic acid 4-phenyl-1-(3-phenyl-propyl)-butyl ester

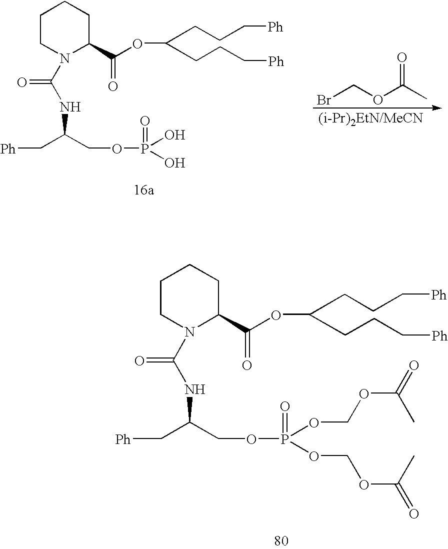

To an acetonitrile solution of the phosphate 16a (10 mg, 0.0158 mmol) at 0° C. was added bromomethyl acetate (15.4 μL, 24.2 mg, 0.158 mmol) and diisopropylethylamine (0.1 mL). After 2 h at 20° C., the solution was concentrated in vacuo. The residue was purified with column chromatography (deactivated by 1% Et₃N in hexanes, eluted with 30% EtOAc in hexanes) to give 5 mg (41% yield) of the title compound 80. ¹H NMR (CDCl₃): δ 7.36-7.06 (15H, m), 5.64 (4H, m), 5.45 (1H, d, J=8.4 Hz), 4.96 (2H, m), 4.22 (1H, m), 4.03 (2H, m), 3.58 (1H, brd), 3.12 (1H, td, J=12.8, 3.3 Hz), 2.99 (1H, dd, J=13.8, 5.7 Hz), 2.79 (1H, dd, J=13.5, 9 Hz), 2.59 (4H, m), 2.19 (1H, br d), 2.13 (3H, s), 2.11 (3H, s); HRMS (MALDI) calc for C₄₁H₅₃N₂O₁₁P (M+H⁺) 803.3279; found 803.3258.

### Example 81

### Acetic acid acetoxymethoxy-(2-{[1-(1-bromo-naphthalen-2-yl)-methanoyl]-amino}-3-phenyl-propoxy)-phosphoryloxymethyl ester

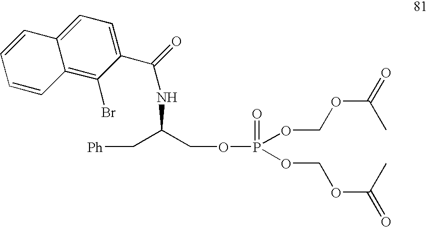

Example 81 was prepared as described in the synthesis of Example 80 using 25-24 (23 mg, 0.05 mmol), bromomethyl acetate (24.5 μL, 38.2 mg, 0.25 mmol) and diisopropylethylamine (0.1 mL). Purification by Et₃N deactivated column chromatography (30% EtOAc in hexanes) gave 3 mg (10% yield) of the title compound 81. ¹H NMR (CDCl₃): δ 8.33 (1H, d, J=8.4 Hz), 7.83 (2H, m), 7.61 (2H, m), 7.41 (1H, dd, J=8.7 Hz), 7.38-7.21 (5H, m), 6.52 (1H, brd, J=9 Hz), 5.64 (4H, m), 4.68 (1H, m), 4.33 (1H, m), 4.18 (1H, m), 3.13 (1H, dd, J=13.8, 6.9 Hz), 3.02 (1H, dd, J=13.5, 8.4 Hz), 2.06 (3H, s), 2.05 (3H, s).

### Example 82

### Acetic acid acetoxymethoxy-{(R)-2-[3-(2-phenoxy-phenyl)-ureido]-3-phenyl-propoxy}-phosphoryloxymethyl ester

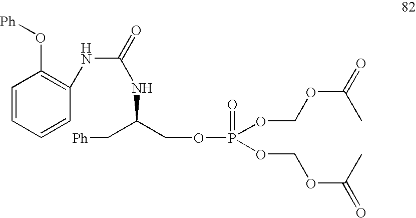

Example 82 was prepared as described in the synthesis of Example 80 using 23b (22 mg, 0.0498 mmol), bromomethyl acetate (24 μL, 0.249 mmol) and diisopropylethylamine (0.05 mL). Purification by Et₃N deactivated column chromatography (50% EtOAc in hexanes) gave 9 mg (31% yield) of the title compound 82. ¹H NMR (CDCl₃): δ 58.22 (1H, dd, J=8.1, 1.5 Hz), 7.36-7.17 (5H, m), 7.14-7.06 (3H, m), 7.01-6.88 (4H, m), 6.82 (1H, dd, J=8.4, 1.8 Hz), 5.65-5.49 (5H, m), 4.28 (1H, m), 4.17 (1H, m), 4.02 (1H, m), 3.01 (1H, dd, J=13.2, 5.7 Hz), 2.81 (1H, dd, J=13.5, 9 Hz), 2.05 (3H, s), 2.04 (3H, s); LCMS (ESP): 609 (M+Na⁺).

### Example 83

### Acetic acid acetoxymethoxy-[2-({1-[5-(3,5dichloro-phenoxy)-furan-2-yl]methanoyl}amino)-3-phenyl-propoxy]-phosphoryloxymethyl ester

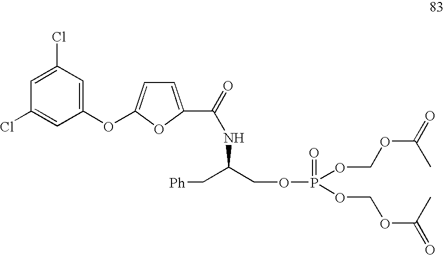

Example 83 was prepared as described in the synthesis of Example 80 using 253 (23 mg, 0.0473 mmol), bromomethyl acetate (23 μL, 0.237 mmol) and diisopropylethylamine (0.049 mL). Purification by Et₃N deactivated column chromatography (50% EtOAc in hexanes) gave 6 mg (20% yield) of the title compound 83. ¹H NMR (CDCl₃): 7.37-7.17 (6H, m), 7.11 (1H, d, J=3.3 Hz), 7.00 (2H, d, 7J=1.5 Hz), 6.67 (1H, br d. J=8.7 Hz), 5.76 (1H, d, J=3.3 Hz), 5.625 (4H, m), 4.5 (1H, m), 4.19 (1H, m), 4.09 (1H, m), 3.02 (1H, dd, J=13.5, 6.3 Hz), 2.93 (1H, dd, J=13.8, 8.1 Hz), 2.11 (3H, s), 2.09 (3H, s).

### Example 84

### Acetic acid acetoxymethoxy-{(R)-3-(3-fluoro-phenyl)-2-[(1-naphthalen-2-yl-methanoyl)-amino]-propoxy}-phosphoryloxymethyl ester

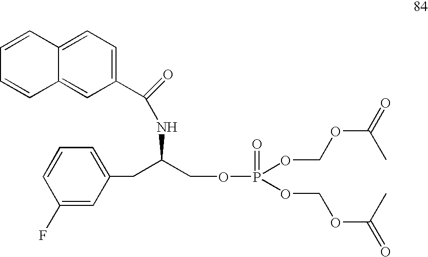

Example 84 was prepared as described in the synthesis of Example 80 using 25-28 (50 mg, 0.124 mmol), bromomethyl acetate (61 μL, 0.620 mmol) and diisopropylethylamine, (0.13 mL). Purification by Et₃N deactivated column chromatography (30-50% EtOAc in hexanes) gave 17 mg (25% yield) of the title compound 84. ¹H NMR (CDCl₃): δ 8.39 (1H, s), 8.00-7.81 (4H, m), 7.56 (1H, m), 7.37-7.23 (2H, m), 7.11 (1H, d, J=7.8 Hz), 7.05 (1H, d, J=9.9 Hz), 6.95 (1H, td, J-8.7, 2.7 Hz), 5.75-5.57 (4H, m), 4.63 (1H, m), 4.30 (1H, m), 4.16 (1H, td, J=10.8, 3.9 Hz), 3.15 (1H, dd, J=13.5, 6 Hz), 2.99 (13.5, 8.7 Hz), 2.12 (3H, s), 2.06 (3H, s); LCMS (ESP): 548 (M+H⁺), 570 (M+Na⁺); HRMS (MALDI) calc for C₂₆H₂₈NO₉FP (M+H⁺) 548.1486; found 548.1489.

### Example 85

### Acetic acid acetoxymethoxy-[(R)-2-[(1-benzo[b]thiophen-2-yl-methanoyl)-amino]-3-(3-fluoro-phenyl)-propoxy]-phosphoryloxymethyl ester

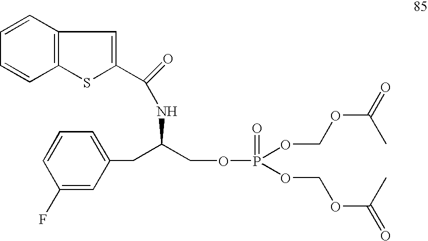

Example 85 was prepared as described in the synthesis of Example 80 using 25-29 (45 mg, 0.11 mmol), bromomethyl acetate (55 μL, 0.55 mmol) and diisopropylethylamine (0.115 mL). Purification by Et₃N deactivated column chromatography (30-50% EtOAc in hexanes) gave 39 mg (64% yield) of the title compound 85. ¹H NMR (CDCl₃): δ 7.92-7.81 (3H, m), 7.48-7.27 (3H, m), 7.09 (1H, br d, J=7.5 Hz), 7.03 (1H, br d, J=9.6 Hz), 6.95 (1H, dd, J=8.7, 2.4 Hz), 5.76-5.59 (4H, m), 4.54 (1H, m), 4.27 (1H, m), 4.13 (1H, td, J=11.1, 3.9 Hz), 3.14 (1H, dd, J=13.8, 6 Hz), 2.96 (1H, dd, J=13.5, 9.3 Hz), 2.15 (3H, s), 2.09 (3H, s); LCMS (ESP): 576 (M+Na⁺); HRMS (MALDI) calc for C₂₄H₂₆NO₉FPS (M+H⁺) 554.1050; found 554.1044.

### Example 86

### Phosphoric acid (R)-2-(5-dimethylamino-naphthalene-1-sulfonylamino)-3-(3-fluoro-phenyl)-propyl ester diphenyl ester

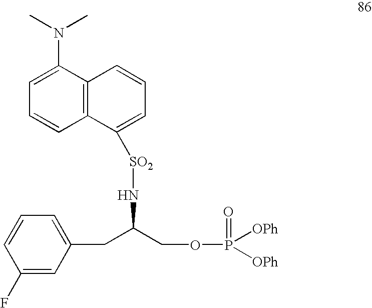

To a THF solution (10 mL) of the alcohol 40 (300 mg, 0.746 mmol) was added Et₃N (0.5 mL), DMAP (30 mg) and diphenyl chlorophosphate (0.23 mL, 301 mg, 1.12 mmol). After 15 h, the solution was diluted by EtOAc (50 mL), washed with brine (2×50 mL), dried and concentrated. The residue was purified by column chromatography to give 310 mg (66% yield) of the title compound 86. ¹H NMR (CDCl₃): δ 8.49 (1H, d, J=8.7 Hz), 8.18 (1H, dd, J=7.5, 1.2 Hz), 8.06 (1H, d, J=8.7 Hz), 7.45 (2H, m), 7.40-7.30 (4H, m), 7.25-7.16 (6H, m), 7.12 (1H, d, J=7.5 Hz), 6.85 (1H, m), 6.60 (1H, td, J=8.4, 2.4 Hz), 6.50 (1H, d, J=7.5 Hz), 6.40 (1H, dt, J=12.3, 1.5 Hz), 5.21 (1H, d, J=8.4 Hz), 4.19 (2H, m), 3.59 (1H, m), 2.87 (6H, s), 2.69 (1H, dd, J=13.5, 7.2 Hz), 2.51 (1H, dd, J=13.8, 7.2 Hz).
Alcohol 88:

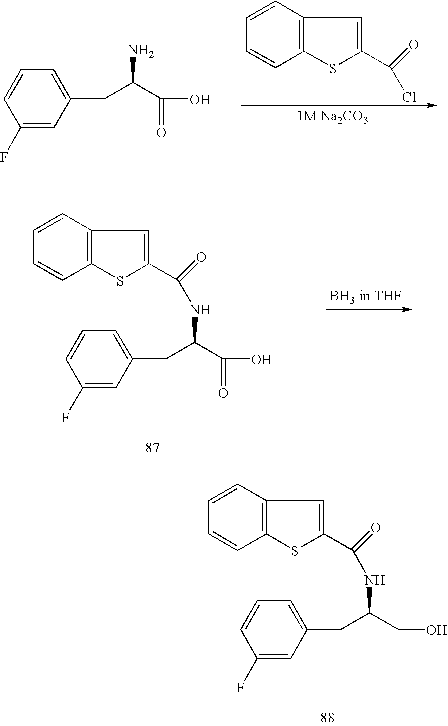

To a 1 M Na₂CO₃ solution (5 mL) at 0° C. was added D-3-fluorophenylalanine (0.5 g, 2.73 mmol) and 1-benzothiophene-2-carbonyl chloride (62 mg, 0.316 mmol). After 15 h at 20° C., the mixture was acidified by addition of ice-cold 5% HCl solution (10 mL). The suspension was extracted with methylene chloride (3×25 mL). The combined organic layers were dried over MgSO₄ and concentrated to yield 0.6 g of 87 as a white solid. The carboxylic acid 87 was dissolved in THF (5 mL). To the THF solution at 0° C. was added 1 M borane in THF (1.31 mL). After 15 h at 25° C., a sat'd NaHCO₃ solution (15 mL) was introduced. The suspension was stirred for 3 h and then extracted with methylene chloride (3×25 mL). The combined organic layers were washed with brine (2×25 mL), dried over Na₂SO₄, and concentrated. Purification by column chromatography (35% EtOAc in hexanes) gave 200 mg (22.5% yield) of the compound 88. ¹H NMR (CDCl₃): δ 7.84 (2H, m), 7.72 (1H, s), 7.41 (2H, m), 7.34-7.25 (1H, m), 7.11-6.89 (3H, m), 6.41 (1H, brd, J=7.5 Hz), 4.37 (1H, m), 3.77 (2H, m), 3.03 (2H, AB), 2.33 (1H, br s).

### Example 89

### Phosphoric acid 2-[(1-benzo[b]thiophen-2-yl-methanoyl)-amino]-3-(3-fluoro-phenyl)-propyl ester diphenyl ester

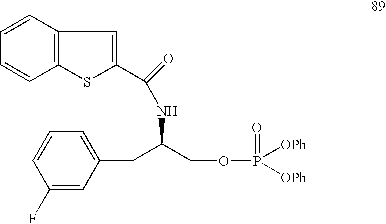

Example 89 was prepared as described in the synthesis of Example 86 using the alcohol 88 (40 mg, 0.122 mmol), Et₃N (0.1 mL), DMAP (4 mg) and diphenyl chlorophosphate (0.29 μL, 37.6 mg, 0.14 mmol). Column chromatography purification (40% EtOAc in hexanes) gave 62 mg (97% yield) of the title compound 89. ¹H NMR (CDCl₃): δ 7.84 (1H, d, J=7.5 Hz), 7.76 (1H, m), 7.66 (1H, s), 7.45-7.32 (5H, m), 7.30-7.13 (8H, m), 7.11-6.98 (2H, m), 6.93 (2H, m), 4.52 (1H, m), 4.40 (1H, m), 4.25 (1H, td, J=11.4, 4.8 Hz), 3.11 (1H, dd, J=13.5, 5.7 Hz), 2.85 (1H, dd, J=13.5, 9.3 Hz); HRMS (MALDI) calc for C₃₀H₂₆NO₅FPS (M+H⁺) 562.1253; found 562.1279.

### Example 90

### 1-[1-Bis-acetoxymethoxy-phospgoryloxymethyl)-2-phenyl-ethylsulfamoyl)]-piperidine-2S-carboxylic acid 4-phenyl-butyl ester

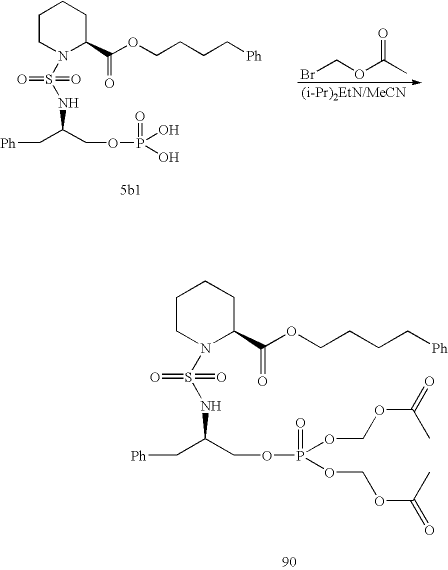

Example 90 was prepared as described in the synthesis of Example 80 using 5b1 (20 mg, 0.036 mmol), bromomethyl acetate (36 μL, 0.36 mmol) and diisopropylethylamine (0.1 mL). Purification by Et₃N deactivated column chromatography (40% EtOAc in hexanes) gave 25 mg (100% yield) of the title compound 90. ¹H NMR (CDCl₃): δ 7.35-7.12 (10H, m), 5.68 (2H, d, J=3 Hz), 5.64 (2H, d, J=2.7 Hz), 5.23 (1H, d, J=9 Hz), 4.61 (1H, d, J=3.9 Hz), 4.26-4.01 (4H, m), 3.81 (1H, m), 3.31 (1H, br d), 2.92 (2H, AB), 2.80 (1H, td, J=12.9, 3.6 Hz), 2.65 (2H, m), 2.20 (1H, br d), 2.13 (6H, s); MS (ESP): 721 (M+Na⁺); 733 (M+Cl)⁻.

### Example 91

### (S)-1-[(R)-1-(Bis-acetoxymethoxy-phosphoryloxymethyl)-2-phenyl-ethylcarbamoyl]-piperidine-2-carboxylic acid

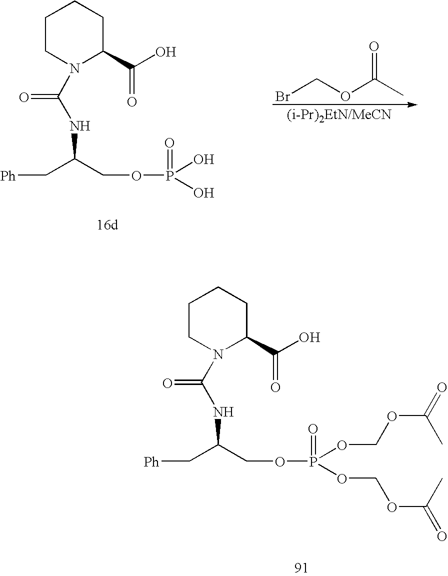

Example 91 was prepared as described in the synthesis of Example 80 using 16d (60 mg, 0.155 mmol), bromomethyl acetate (0.15 μL, 1.55 mmol) and diisopropylethylamine (0.4 mL, 2.33 mmol). Purification by Et₃N deactivated column chromatography (40% EtOAc in hexanes) gave 45 mg (55% yield) of the title compound 91. ¹H NMR (CDCl₃): δ 7.32-7.07 (5H, m), 5.58 (2H, d, J=1.8 Hz), 5.54 (2H, d, J=1.8 Hz), 4.68-4.50 (2H, m), 4.22 (1H, m), 3.98 (1H, dd, J=13.8, 5.1 Hz), 3.55 (1H, dd, J=12, 4.2 Hz), 3.16 (1H, dd, J=13.8, 9.3 Hz), 3.02 (1H, dd, J=13.8, 6 Hz), 2.69 (1H, td, J=13.2, 3.3 Hz), 2.10 (3H, s), 2.10 (3H, s).

### Example 92

### Acetic acid acetoxymethoxy-[(R)-2-[(7-diethylamino-2-oxo-2H-chromene-3-carbonyl)-amino]-3-(3-fluoro-phenyl)-propoxy]-phosphoryloxymethyl ester

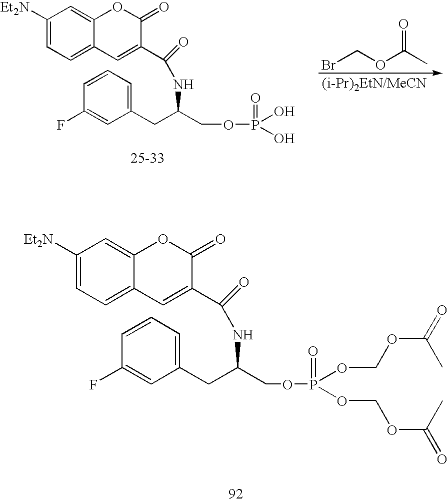

Example 92 was prepared as described in the synthesis of Example 80 using Example 25-33 (18 mg, 0.0366 mmol), bromomethyl acetate (0.03 mL, 0.3 mmol) and diisopropylethylamine (0.1 mL, 0.6 mmol). Purification by Et₃N deactivated column chromatography (100% EtOAc in hexanes) gave 5 mg (25% yield) of the title compound 92. ¹H NMR (CDCl₃): δ9.05 (1H, d, J=8.3 Hz), 8.64 (1H, s), 7.41 (1H, t, J=9.1 Hz), 7.26 (1H, m), 7.08 (1H, d, J=7.5 Hz), 7.02 (1H, br d, J=9.8 Hz), 6.92 (1H, td, J=8.3, 2.1 Hz), 6.64 (1H, dd, J=9.3, 2.7 Hz), 6.49 (1H, d, J=2.2 Hz), 5.67 (1H, dd, J=13.6, 0.9 Hz), 4.55 (1H, br s), 4.17 (2H, m), 3.46 (4H, q, J=7 Hz), 3.01 (1H, d, J=7.3 Hz), 2.13 (6H, s), 1.24 (6H, t, J=7.2 Hz).

### Example 93

### Acetic acid acetoxymethoxy-[3-[(benzo[b]thiophene-2-carbonyl)-amino]4-(3-fluoro-phenyl)-butyl]-phosphinoyloxymethyl ester

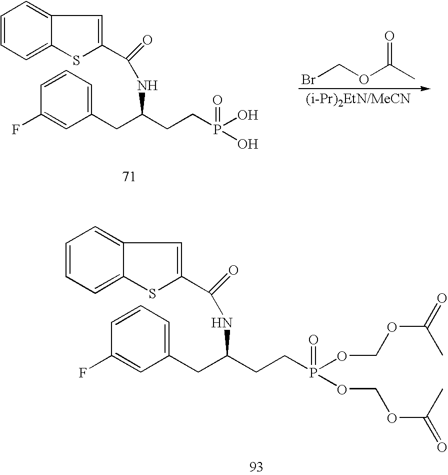

Example 93 was prepared as described in the synthesis of Example 80 using Example 71 (22 mg, 0.0541 mmol), bromomethyl acetate (0.05 mL, 0.52 mmol) and diisopropylethylamine (0.13 mL, 0.77 mmol). Purification by flash column chromatography (100% EtOAc in hexanes) gave 23 mg (77% yield) of the title compound 93. ¹H NMR (CDCl₃): δ 7.88-7.79 (3H, m), 7.41 (2H, m), 7.27 (1H, m), 7.02 (1H, d, J=7.6 Hz), 6.94 (2H, m), 6.80 (1H, d, J=8.6 Hz), 5.71-5.55 (4H, m). 4.38 (1H, br 10 t, J=7.3 Hz), 3.05 (1H, dd, J=13.7, 6.1 Hz), 2.87 (1H, dd, J=13.7, 7.1 Hz), 2.12 (3H, s), 2.05 (3H, s). 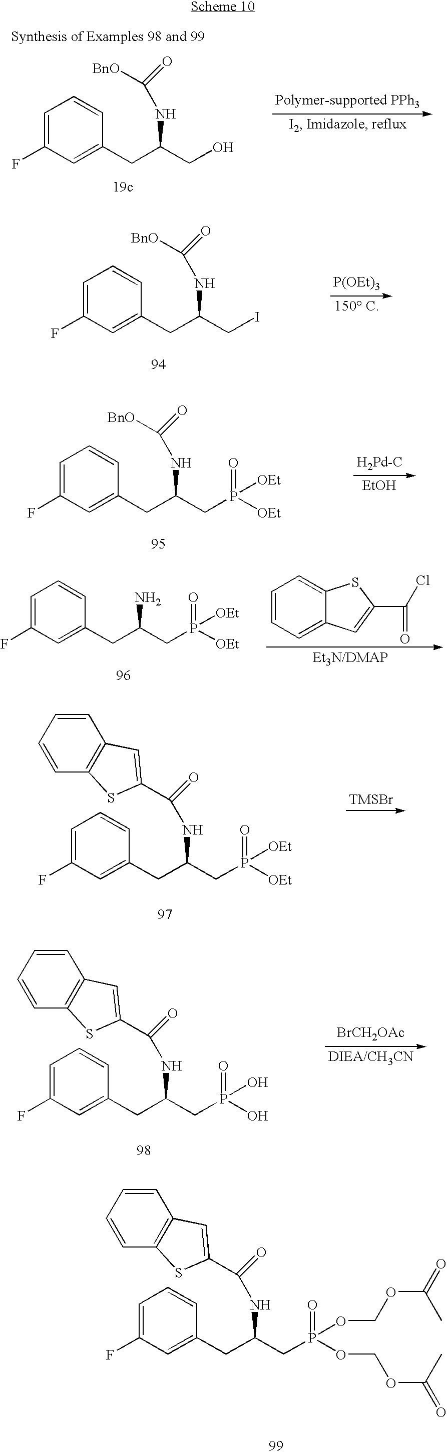
Synthesis of Example 100 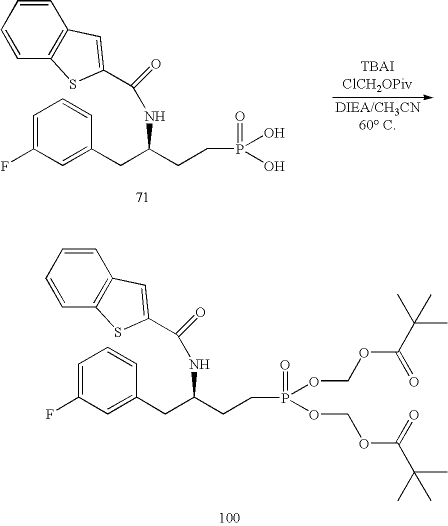
Phosphonate Ester 95:

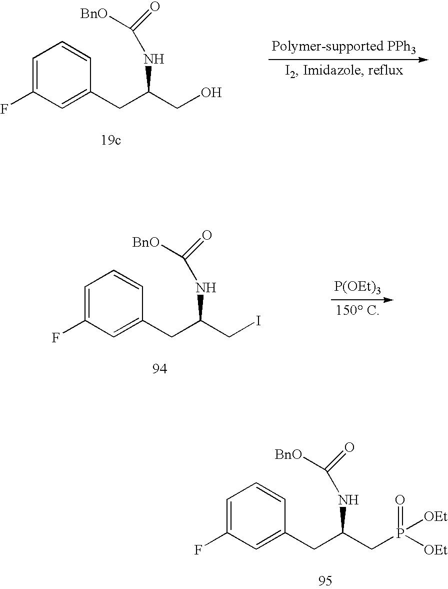

To a dry CH₂Cl₂ (20 mL) suspension of polymer-supported triphenylphosphine (1.69 g, 4.53 mmol) was added iodine (1.15 g, 4.53 mmol). After 15 min, imidazole (0.33 g, 5.15 mmol) was added. The suspension was stirred for another 15 min. A CH₂Cl₂ (8 mL) solution of 19c (600 mg, 2.06 mmol) was added. The mixture was heated at reflux for 1 h. After cooling the mixture to 25° C., the polymeric solid was filtered off. The filtrate was washed with Na₂S₂O₃ solution (concentrated, 2×30 mL), water (1×25 mL) and brine (1×25 mL). All the solvent was removed in vacuo, affording 914 mg (100%) of the iodide 94 as a yellow solid. A portion of the iodide 94 (420 mg) was mixed with triethyl phosphite (2.5 mL) in a sealed tube. The suspension was heated at 150° C. for 30 min by microwave radiation. The triethyl phosphite was removed in vacuo. The residue was purified by column chromatography (30-50% EtOAc in hexanes) to give 80 mg (19% yield) of the phosphonate 95 as a colorless oil. ¹H NMR (CDCl₃): δ 7.32-7.10 (5H, m), 6.95-6.82 (4H, m), 5.43 (1H, d, J=6 Hz), 5.01 (2H, s), 4.12-3.92 (4H, m), 3.01-2.75 (2H, m), 1.92 (2H, m), 1.28-1.13 (6H, m); LCMS (positive APCI): 424 (M+H⁺), 446 (M+Na⁺).
Amine 96:

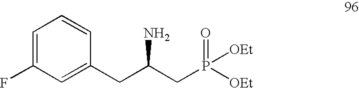

To an ethanol solution (5 mL) of compound 95 (235 mg, 0.556 mmol) was added palladium on carbon (10%, 40 mg). The suspension was kept under hydrogen (1 atm) for 15 h. After filtration, the filtrate was concentrated. The residue was purified by column chromatography (MeOH/CH₂Cl₂ 5/95) to afford 148 mg (92% yield) of the compound 96 as an oil. ¹H NMR (DMSO-d₆): δ 7.4-7.0 (4H, m), 4.05-3.90 (4H, m), 3.25 (1H, m), 2.78 (1H, dd, J=13.4, 6.1 Hz), 2.69 (1H, dd, J=12.2, 6.1 Hz), 2.06 (2H, br s), 1.9-1.70 (2H, m). LCMS (positive APCI): 290 (M+H⁺), 312 (M+Na⁺).
Amide 97:

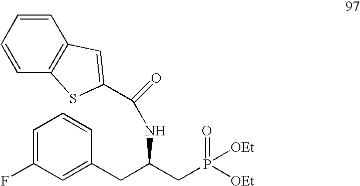

To a methylene chloride solution (5 mL) of the amine 96 (144 mg, 0.498 mmol) was added triethylamine (0.139 mL, 0.996 mmol), 4-(dimethylamino)pyridine (6 mg, 0.0498 mmol), and 1-benzothiophene-2-carbonyl chloride (123 mg, 0.623 mmol) at 0° C. After 15 h at 25° C., the mixture was diluted with methylene chloride (20 mL), washed with ice-cold HCl solution (1 M, 1×20 mL), sodium carbonate solution (1 M, 1×20 mL), and brine (1×20 mL). The solution was then dried (Na₂SO₄) and concentrated. The residue was purified by column chromatography (30-50% EtOAc in hexanes) to give 150 mg (67% yield) of the compound 97 as a white solid. ¹H NMR (CDCl₃): δ 7.85-7.65 (3H, m), 7.33 (2H, m), 7.21-7.15 (1H, m), 7.03 (1H, d, J=7.7 Hz), 6.96 (1H, br d, J=9.9 Hz), 6.88 (1H, td, J=8.1, 1.9 Hz), 4.51 (1H, d, J=2.1 Hz), 4.16-3.97 (4H, m), 3.17 (1H, dd, J=12, 5.1 Hz), 2.91 (1H, dd, J=13.4, 8.9 Hz), 2.02 (1H, d, J=4.7 Hz), 1.98 (1H, d, J=5.3 Hz), 1.33 (3H, t, J=6.9 Hz), 1.73 (3H, t, J=7.1 Hz); LCMS (ESP): 450 (M+H⁺), 472 (M+Na⁺); 448 (M−H).

### Example 98

### [2-[(Benzo[b]thiophene-2-carbonyl)-amino]-3-(3-fluoro-phenyl)-propyl]-phosphonic acid

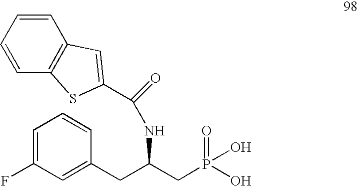

To a methylene chloride solution (2 mL) of phosphonate ester 97 (140 mg, 0.31 mmol) was added bromotrimethylsilane (1 mL). After 15 h, the solution was concentrated in vacuo. The oily residue was triturated with water (3×2 mL). In the process, a white solid 98 (110 mg, 89% yield) was obtained by filtration. ¹H NMR (DMSO-d₆): δ 8.66 (1H, d, J=8.4 Hz), 8.1-7.92 (3H, m), 7.45 (2H, m), 7.32 (1H, q, J=8 Hz), 7.12-7.0 (3H, m), 4.42 (1H, m), 3.14 (1H, dd, J=13.7, 2.1 Hz), 2.94 (1H, dd, J=13.4, 8.1 Hz), 1.92 (2H, m); LCMS (positive APCI): 394 (M+H⁺), 416 (M+Na⁺); Elemental Analysis for (C₁₀H₁₇NO₄ PFS 0.3H₂O) calc: C, 54.21; H, 4.45; N, 3.51; found: C, 54.15; H, 4.47; N, 3.47.

### Example 99

### Acetic acid acetoxymethoxy-[2-[benzo[b]thiophene-2-carbonyl)-amino]-3-(3-fluoro-phenyl)-propyl]-phosphinoyloxymethyl ester

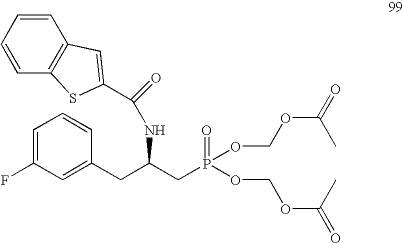

To an acetonitile solution (1 mL) of the phosphonate acid 98 (31.8 mg, 0.0809 mmol) was added diisopropylethylamine (0.127 mL, 0.728 mmol) and bromomethyl acetate (60 μL, 0.607 mmol) at 0° C. After 15 h at 25° C., the solution was concentrated and the resulting residue was purified by column chromatography (50-70% EtOAc in hexanes), affording 26 mg (59% yield) of the title compound 99 as a white solid. ¹H NMR (CD₃OD): δ 7.85-7.75 (3H, m), 7.33 (2H, m), 7.19 (1H, q, J=8 Hz), 6.99 (1H, d, J=7.5 Hz), 6.94 (1H, dt, J=7.6, 2.2 Hz), 6.84 (1H, td, J=8.3, 2.5 Hz), 5.6-5.45 (4H, m), 4.50 (1H, m), 2.93 (2H, m), 2.35-2.16 (2H, m), 1.94 (3H, s), 1.94 (2H, s); HRMS (MALDI) calc for C₂₄H₂₆NO₈FPS (M+H⁺) 538.1129; found 538.1101.

### Example 100

### 2,2-Dimethyl-propionic acid[3-[(benzo[b]thiophene-2-carbonyl)-amino]-4-(3-fluoro-phenyl)-butyl]-(2,2-dimethyl-propionyloxymethoxy)-phosphinoyloxymethyl ester

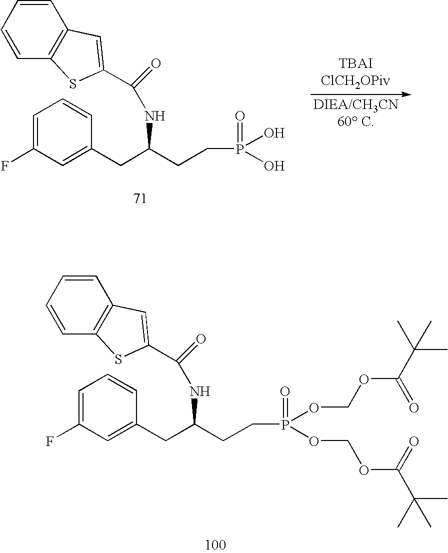

To an acetonitrile solution (5 mL) of Example 71 (50 mg, 0.123 mmol) was added tetrabutylamonium iodide (5 mg), diisopropylethylamine (0.2 mL), and chloromethyl pivalate (132 μL, 0.92 mmol) at 0° C. The solution was heated at 60° C. for 4 h and concentrated in vacuo. The residue was purified by column chromatography (35% EtOAc in hexanes), affording 20 mg (26% yield) of the title compound 100. ¹H NMR (CD₃OD): δ7.81-7.75 (3H, m), 7.32 (2H, m), 7.16 (1H, q, J=8 Hz), 6.97 (1H, d, J=7.8 Hz), 6.92 (1H, dt, J=10.1, 2.3 Hz), 6.81 (1H, td, J=8.3, 2 Hz), 5.6-5.48 (4H, m), 4.24 (1H, m), 2.84 (2H, m), 1.95-1.7 (4H, m), 1.09 (9H, s), 1.04 (9H, s); LCMS (positive APCI): 636 (M+H⁺); HRMS (MALDI) calc for C₃₁H₄₀NO₈FPS (M+H⁺) 636.2196; found 636.2182.

### Biological Testing: Pin1 Peptidyl-Prolyl Isomerase Assay

PIN1 is a phosphorylation dependent peptidyl-prolyl isomerase. The PIN1 assay is a spectrophotometric assay based on the coupled chymotrypsin or subtilisin catalyzed, cis-trans conformation dependent cleavage of a para-nitroanaline containing peptide substrate. This improved general rotamase assay was first described by Kofron, et al. (Biochemistry, 30, 6217-6134 (1991)) and applied to PIN1 isomerase activity by Yaffe, et al. (Science, 278, 1957-1960 (1997)). Cleavage of the isomerized peptide releases para-nitroanaline, which can be monitored by an increase in absorbance at 390 nm. The PIN1 peptide substrate, succinyl-leucine-proline-phenylalanine-paranitroaniline (Suc-AEPF-pNA) (Bachem), is kept in a predominantly cis conformation with an anhydrous TFE/LiCl solvent mixture.

Upon dilution into an aqueous assay mixture containing PIN1, the peptide substrate undergoes PIN1 catalyzed isomerization to the trans conformation. Chymotrypsin or subtilisin (subtilisin Carlsberg protease, available from Sigma, catalog number P-5380) cleaves the trans product to form free para-nitroanaline. To minimize the spontaneous isomerization of the peptide substrate, reactions are performed at 15° C. A typical reaction contains 25 mM MOPS pH 7.5, 0.5 mM TCEP, 2% DMSO, 5 μl of a 25 mg/ml solution of subtilisin Carlsberg, 50 nM PIN1-PPiase, and 100 μM Suc-AEPF-pNA peptide substrate. Reactions are cooled to 15° C. and initiated with the addition of Suc-AEPF-pNA. The absorbance at 390 nm is monitored continuously until all substrate has been converted to the cleaved product. This data, the progress curve, is then fitted to an exponential equation to determine a rate constant k for the reaction. The rate constant k is linearly proportional to the concentration of active enzyme present in the assay mixture once the rate constant for the spontaneous isomerization is subtracted. The Kₘ for this substrate is much higher than 100 μM ([S]<<Kₘ). Therefore, during inhibition experiments, the IC₅₀, for non-tight binding inhibitors, is essentially the inhibition constant Kᵢ.

In Table 1, the Kᵢ data reported under the PIN1—CD heading corresponds to testing with PIN1 peptide containing the catalytic peptidyl-prolyl isomerase domain but devoid of the PIN1 WW domain. Similarly, the dissociation constant (K_{d}) data under PIN1-CD refers to testing with a peptide containing the catalytic PIN1 domain but devoid of the PIN1 WW domain.

TABLE 1

| | PIN1-CD | PIN1-CD |
|---|---|---|
| Example No. | K_{d} (μM) | Kᵢ (μM) |
| 3a | | |
| 3b1 | | |
| 3b2 | | |
| 3b3 | | |
| 3b4 | | |
| 5b1 | <10 | <10 |
| 5b2 | <10 | <10 |
| 5b5 | <10 | <10 |
| 14a | <100 | |
| 14b | | |
| 16a | <10 | <1 |
| 16c | <10 | |
| 16d | <10 | <10 |
| 22e | | |
| 23a | <10 | |
| 23b | <1 | |
| 23c | <1 | |
| 23d | <1 | |
| 25-1 | <1 | |
| 25-2 | <1 | |
| 25-2′ | <1 | |
| 25-3 | <1 | |
| 25-4 | <1 | |
| 25-5 | <1 | |
| 25-6 | <1 | |
| 25-7 | <1 | |
| 25-8 | | <1 |
| 25-9 | | |
| 25-10 | <1 | |
| 25-11 | <1 | |
| 25-12 | <1 | |
| 25-13 | <10 | |
| 25-14 | <10 | |
| 25-15 | | <10 |
| 25-16 | | <1 |
| 25-40 | | <10 |
| 25-17 | | |
| 25-18 | <1 | |
| 25-19 | <1 | |
| 25-20 | <1 | |
| 25-21 | <1 | |
| 25-22 | <1 | |
| 25-23 | <1 | <1 |
| 25-24 | <1 | |
| 25-25 | <1 | |
| 25-26 | <10 | |
| 25-27 | <10 | |
| 25-28 | <1 | <1 |
| 25-29 | | <1 |
| 25-30 | <1 | <1 |
| 25-31 | <1 | <1 |
| 25-32 | <1 | <1 |
| 25-33 | | <1 |
| 25-34 | <1 | <1 |
| 25-35 | <1 | <1 |
| 25-36 | <1 | <1 |
| 25-37 | <1 | <1 |
| 25-38 | | |
| 30 | <10 | <100 |
| 25-39 | | |
| 33 | | <10 |
| 37 | <2 | <10 |
| 42 | | <10 |
| 49 | <1 | <1 |
| 54 | <1 | <1 |
| 58 | <10 | |
| 61 | | <1 |
| 66 | | <1 |
| 67 | | <1 |
| 71 | | <1 |
| 72 | <100 | |
| 73 | | |
| 74 | <1 | <1 |
| 79 | | <1 |
| 82 | | |
| 83 | | |
| 85 | | <100 |
| 86 | | <100 |
| 89 | | 20% |
| | | @ 20 mM |
| 91 | | 0% |
| | | @ 100 mM |
| 98 | | <1 |

The exemplary compounds described above may be formulated into pharmaceutical compositions according to the following general examples.

### Example 1

### Parenteral Composition

To prepare a parenteral pharmaceutical composition suitable for administration by injection, 100 mg of a water-soluble salt of a compound of Formula I is dissolved in DMSO and then mixed with 10 mL of 0.9% sterile saline. The mixture is incorporated into a dosage unit form suitable for administration by injection.

### Example 2

### Oral Composition

To prepare a pharmaceutical composition for oral delivery, 100 mg of a compound of Formula I is mixed with 750 mg of lactose. The mixture is incorporated into an oral dosage unit for, such as a hard gelatin capsule, which is suitable for oral administration.

1. A compound of the Formula I: 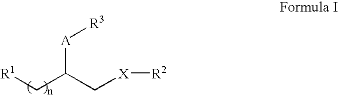
wherein
n is 1 or 2;
A is a divalent —CH═CH—, —(C₁-C₇-alkyl)-Y—, —NR^{d}(CH₂)ₜ Y—, —Y—(C₁-C₇-alkyl)-, —Y—(C₁-C₇ alkyl)-, —Y—NH—, —Y—NR^{d}(C—C₆-alkyl)-, —S—, —S(O)₂—, —O—Y—, —Y—O—, —Y—S—, or —S—Y—, wherein R^{d} is H or C₁-C₆ alkyl, t is an integer from 0 to 5, Y is C(O), C(S), S(O), S(O)₂, or a bond;
X is a direct bond, CH₂, CF₂, O, S, NH, C(O), or C(S);
R¹ is a C₃-C₁₀ cycloalkyl, 4-10 membered heterocycloalkyl, C₆-C₁₀ aryl, or 4-10 membered heteroaryl group, wherein R¹ is unsubstituted or substituted with 1 to 4 R¹⁰ groups;
R² is —S(O)₂OH, —S(O)₂NR^{d}R^{e}, or —P(O)(OR⁴)₂, wherein R⁴ is an H, C₁-C₁₀-alkyl, C₆-C₁₀ aryl, or —CH₂—O—C(O)R^{e}CH₃ group, R^{d} and R^{e} are each independently an H or C₁-C₆ alkyl group, and R⁴ is unsubstituted or substituted with 1 to 4 R¹⁰ groups; and
R³ is OH, C₁-C₇-alkyl, C₁-C₇-alkoxyl, C₆-C₁₀ aryl, 4-10 membered heteroaryl, C₃-C₁₀ cycloalkyl, 3-10 membered heterocycloalkyl, —NH(R⁵), or —N(R⁵)₂ group, wherein R⁵ is independently selected from H, C₁-C₇ alkyl, C₆-C₁₀ aryl, or 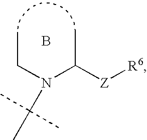
wherein ring B is a 5- or 6-membered heterocycloalkyl group, Z is a divalent C(O)Z′, heteroaryl or heterocycloalkyl group wherein Z′ is a divalent O, S, NH, N(CH₃), CO₂, or CH₂, and R⁶ is H, C₁-C₁₀ alkyl, aryl, C₁-C₆ alkyl-aryl, or arylalkyl group, wherein R³, R⁵, B and R⁶ are unsubstituted or substituted with 1 to 4 R¹⁰ groups;
wherein each R¹⁰ is independently selected from halo, amino, ═O, ═S, ═NH, cyano, nitro, hydroxyl, —SH, haloalkyl, 2-10 membered heteroalkyl, C₁-C₆ alkoxy, C, C₁₀ alkyl, C₂-C₆ alkenyl, C₂-C₆ alkynyl, —C(O)ⱼR⁸, C(O)ⱼR^{d}, OC(O)OC(O)R^{d}, —OOH, —C(NR^{d})NR^{b}R^{c}, —NR^{d}C(NR^{e})NR^{b}R^{c}, —NR^{d}C(O)ⱼR^{b}, —C(O)NR^{b}R^{c}, —C(O)NR^{d}COR^{b}, —OC(O)NR^{b}R^{c}, —NR^{b}R^{c}, —NR^{d}OR^{c}, —C(S)NR^{b}R^{c}, —NR^{d}C(S)NR^{b}R^{c}, —NR^{d}C(O)NR^{b}R^{c}, —OSH, —S(O)ⱼR^{b}, —OS(O)ⱼR^{b}, —SC(O)R^{b}, —S(O)ⱼC(O)OR^{b}, —SCOR^{d}, —NR^{d}SR^{c}, —SR^{b}, —NHS(O)ⱼR^{b}, —COSR^{b}, —C(O)S(O)ⱼR^{b}, —CSR^{b}, —CS(O)ⱼR^{b}, —C(SO)OH, —C(SO)₂OH, —NR^{d}C(S)R^{c}, —OC(S)R^{b}, —OC(S)OH, —OC(SO)₂R^{b}, —S(O)ⱼNR^{b}R^{c}, —SNR^{b}R^{c}, —S(O)NR^{b}R^{c}, —NR^{d}CS(O)ⱼR^{c}, —C(O)ⱼ(CH₂)ₜNR^{d}(4-10 membered heteroaryl), —C(O)ⱼ(CH₂)ₜNR^{d}(4-10 membered heterocycloalkyl), —(CR^{d}R^{e})ₜCN, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})_{q}C(O)(CR^{d}R^{e})ₜ(C₃-C₁₀ cycloalkyl), —(CR^{d}R^{e})_{q}C(O)(CR^{d}R^{e})ₜ(C₆-C₁₀ aryl), (CR^{d}R^{e})_{q}C(O)(CR^{d}R^{e})ₜ(4-10 membered heterocycloalkyl), —(CR^{d}R^{e})_{q}C(O)(CR^{d}R^{e})ₜ(4-10 membered heteroaryl), —(CR^{d}R^{e})ₜ(CR^{d}R^{e})_{q}(C₃-C₁₀ cycloalkyl), —(CR^{d}R^{e})ₜ(CR^{d}R^{e})_{q}(C₆-C₁₀ aryl), —(CR^{d}R^{e})ₜO(CR^{d}R^{e})_{q}(4-10 membered heterocycloalkyl), —(CR^{d}R^{e})ₜO(CR^{d}R^{e})_{q}(4-10 membered heteroaryl), —(CR^{d}R^{e})_{q}SO₂(CR^{d}R^{e})ₜ(C₃-C₁₀ cycloalkyl), —(CR^{d}R^{e})_{q}SO₂(CR^{d}R^{e})ₜ(C₆-C₁₀ aryl), —(CR^{d}R^{e})_{q}SO₂(CR^{d}R^{e})ₜ(4-10 membered heterocycloalkyl), and —(CR^{d}R^{e})_{q}SO₂(CR^{d}R^{e})ₜ(4-10 membered heteroaryl), wherein R^{e} is selected from the group consisting of halo, hydroxyl, —NR^{d}R^{e}, C₁-C₁₀ alkyl, haloalkyl, C₁-₆ alkoxyl, R^{b} and R^{c} are independently selected from H, C₁-C₁₀ alkyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), and —(CR^{d}R^{e})ₜ(4-10 membered heteroaryl), R^{d} and R^{e} are independently H or C₁-C₆ alkyl, j is an integer from 0 to 2, q and t are each independently an integer from 0 to 5, and 1 or 2 ring carbon atoms of the cyclic moieties of the foregoing R¹⁰ groups are unsubstituted or substituted with ═O, and the alkyl, alkenyl, alkynyl, aryl and cyclic moieties of the foregoing R¹⁰ groups are unsubstituted or substituted with 1 to 3 substituents independently selected from halo, ═O, cyano, nitro, —(CR^{d} R e)ₜCN, haloalkyl, 2-10 membered heteroalkyl, —OR^{b}, —C(O)ⱼR^{b}, —NR^{d}C(O)R^{b}, —C(O)NR^{b}R^{c}, —NR^{b}R^{c}, —NR^{b}OR^{c}, —NR^{d}C(O)ⱼNR^{b}R^{c}, —NR^{d}C(O)ⱼR^{b}R^{c}, —OC(O)ⱼR^{b}, OC(O)NR^{b}R^{c}, SR^{d}, C₁-C₁₀ alkyl, C₂-C₆ alkenyl, C₂-C₆ alkynyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})ₜ(C₆-C₁₀ aryl)-(C₁-C₆ alkyl); wherein t, R b, R^{c}, R^{d}, R^{e} are as defined above;
or a pharmaceutically acceptable prodrug of said compound, pharmaceutically active metabolite of said compound, or pharmaceutically acceptable salt of said compound or metabolite.

2. A pharmaceutically acceptable salt according to claim 1.

3. A compound or pharmaceutically acceptable salt according to claim 1, wherein:
n is 1 or 2;
A is a divalent —NH—Y—, —NR^{d}(CH₂)ᵣY—, or —O—Y—, and Y is C(O) or S(O)₂;
X is a direct bond, CH₂, O, or S;
R¹ is a C₆-C₁₀ aryl or 4-10 membered heteroaryl group unsubstituted or substituted with 1 to 4 R¹⁰ groups;
R² is —S(O)₂OH, or —P(O)(OR⁴)₂, wherein R⁴ is an H, C₁-C₁₀ alkyl, or C₆-C₁₀ aryl group, and is unsubstituted or substituted with 1 to 4 R¹⁰ groups; and
R³ is a C₆-C₁₀ aryl, 4-10 membered heteroaryl, —NH(C₆H₅), or 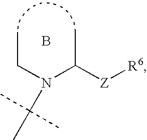
wherein ring B is a 5- or 6-membered heterocycloalkyl group, Z is a divalent C(O)Z′, heteroaryl or heterocycloalkyl group wherein Z′ is a divalent O, S, NH, N(CH₃), CO₂, or CH₂, and R⁶ is H or a C₁-C₁₀ alkyl group, wherein R³, B, and R⁶ is unsubstituted or substituted with 1 to 4 R¹⁰ groups;
wherein each R¹⁰ is independently selected from halo, amino, ═O, ═S, ═NH, cyano, nitro, hydroxyl, —SH, haloalkyl, 2-10 membered heteroalkyl, C₁-C₆ alkoxy, C₁-C₁₀ alkyl, C₂-C₆ alkenyl, C₂-C₆ alkynyl, —C(O)ⱼR^{d}, —OC(O)ⱼR^{d}, —OC(O)OC(O)R^{d}, —OOH, —C(NR^{d})NR^{b}R^{c}, —NR^{d}C(NR^{e})NR^{b}R^{c}, —NR C(O)ⱼR^{b}, —C(O)NR^{b}R^{c}, —C(O)NR^{d}COR^{b}, —OC(O)NR^{b}R^{c}, —NR^{b}R^{c}, —NR^{d}OR^{c}, —C(S)NR^{b}R^{c}, —NR C(S)NR^{b}R^{c}, —NR^{d}C(O)NR^{b}R^{c}, —OSH, —S(O)ⱼR^{b}, —OS(O)ⱼR^{b}, —SC(O)R^{b}, —S(O)ⱼC(O)OR^{b}, —SCOR^{d}, —NR^{d}SR^{c}, —SR^{b}, —NHS(O)ⱼR^{b}, —COSR^{b}, —C(O)S(O)ⱼR^{b}, —CSR^{b}, —CS(O)ⱼR^{b}, —C(SO)OH, —C(SO)₂OH, —NR^{d}C(S)R^{c}, —OC(S)R^{b}, —OC(S)OH, —OC(SO)₂R^{b}, —S(O)ⱼNR^{b}R^{c}, —SNR^{b}R^{c}, —S(O)NR^{b}R^{c}, —NR^{d}CS(O)ⱼR^{c}, —C(O)ⱼ(CH₂)ₜNR^{d}(4-10 membered heteroaryl), —C(O)ⱼ(CH₂)ₜNR^{d}(4-10 membered heterocycloalkyl), —(CR^{d}R^{e})ₜCN, —(CR^{d} R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})_{q}C(O)(CR^{d}R^{e})ₜ(C₃-C₁₀ cycloalkyl), —(CR^{d}R^{e})_{q}C(O)(CR^{d}R^{b})ₜ(C₆-C₁₀ aryl), —(CR^{d}R^{c})_{q}C(O)(CR^{d}R^{e})ₜ(4-10 membered heterocycloalkyl), (CR^{d}R^{e})_{q}C(O)(CR^{d}R^{b})ₜ(4-10 membered heteroaryl), —(CR dR e)ₜO(CR^{d}R^{e})_{q}(C₃-C₁₀ cycloalkyl), (CR^{d}R^{e})ₜO(CR^{d}R^{e})_{q}(C₆-C₁₀ aryl), —(CR^{d}R^{e})ₜO(CR^{d}R^{e})_{q}(4-10 membered heterocycloalkyl), —(CR^{d}R^{e})_{q}(CR^{d}R^{e}(4-10 membered heteroaryl), —(CR^{d}R^{e})_{q}SO₂(CR^{d}R^{b})ₜO(C₃-C₁₀ cycloalkyl), —(CR^{d}R^{e})_{q}SO₂(CR^{d}R^{b})ₜ(C₆-C₁₀ aryl), —(CR^{d}R^{e})SO₂(CR^{d}R^{e})ₜ(4-10 membered heterocycloalkyl), and —(CR^{d}R^{e})_{q}SO₂(CR^{d}R^{b})ₜ(4-10 membered heteroaryl), wherein R” is selected from the group consisting of halo, hydroxyl, —NR^{d}R^{e}, C₁-C₁₀ alkyl, haloalkyl, C₁-C₆ alkoxyl, R^{b} and R^{c} are independently selected from H, C₁-C₁₀ alkyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), and —(CR^{d}R^{e})ₜ(4-10 membered heteroaryl), R^{d} and R^{e} are independently H or C₁-C₈ alkyl, j is an integer from 0 to 2, q and t are each independently an integer from 0 to 5, and 1 or 2 ring carbon atoms of the cyclic moieties of the foregoing R¹⁰ groups are unsubstituted or substituted with ═O, and the alkyl, alkenyl, alkynyl, aryl and cyclic moieties of the foregoing R¹⁰ groups are unsubstituted or substituted with 1 to 3 substituents independently selected from halo, ═O, cyano, nitro, —(CR^{d}R^{b})ₜCN, haloalkyl, 2-10 membered heteroalkyl, —OR^{b}, —C(O)ⱼR^{b}, —NR^{d}DC(O)R^{b}, —C(O)NR R^{c}, —NR^{b}R^{c}, —NR^{b}OR_{c}, —NR^{d}C(O)ⱼNR^{b}R^{c}, —NR^{d}C(O)ⱼR^{b}R^{c}, —OC(O)ⱼR^{b}, —OC(O)NR^{b}R^{c}, —SR^{d}, C₁-C₁₀ alkyl, C₂-C₆ alkenyl, C₂-C₆ alkynyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})ₜ(C₆-C₁₀ aryl)-(C₁-C₆ alkyl); and wherein t, R^{b}, R^{c}, R^{d}, R^{e} are as defined above.

4. A compound or pharmaceutically acceptable salt according to claim 3, wherein:
n is 1;
A is a divalent —NH—Y— or —O—Y—, wherein Y is C(O);
X is a direct bond, CH₂, or O;
R¹ is a C₆-C₁₀ aryl group unsubstituted or substituted with 1 to 4 R¹⁰ groups;
R² is —P(O)(OR⁴)₂, wherein R is an H, C₁-C₁₀alkyl, or C₆-C₁₀ aryl group, and is unsubstituted or substituted with 1 to 4 R¹⁰ groups; and
R³ is a C₆-C₁₀ aryl, 4-10 membered heteroaryl, or 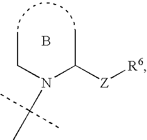
wherein ring B is an unsubstituted 6-membered heterocycloalkyl, Z a divalent C(O)Z′, Z′ is a divalent O, S, or CH₂, and R⁶ is a C₁-C₁₀ alkyl group, wherein R³, B and R⁶ are unsubstituted or substituted with 1 to 4 R¹⁰ groups;
wherein each R¹⁰ is independently selected from halo, amino, ═O, ═S, ═NH, cyano, nitro, hydroxyl, —SH, haloalkyl, 2-10 membered heteroalkyl, C₁-C₆ alkoxy, C₁-C₁₀ alkyl, C₂-C₆ alkenyl, C₂-C₆ alkynyl, —C(O)ⱼR^{a}, —OC(O)ⱼR^{d}, —OC(O)OC(O)R^{d}, —OOH, —C(NR^{d})NR^{b}R^{c}, —NR^{d}C(NR^{e})NR^{b}R^{c}, —NR^{d}C(O)ⱼR^{b}, —C(O)NR^{b}R^{c}, —C(O)NR^{d}COR^{b}, —OC(O)NR^{b}R^{c}, —NR^{b}R^{c}, —NR^{d}OR^{c}, —C(S)NR^{b}R^{c}, —NR^{d}C(S)NR R^{c}, —NR^{d}C(O)NR R^{c}, —OSH, —S(O)ⱼR^{b}, OS(O)ⱼR^{b}, —SC(O)R^{b}, —S(O)ⱼC(O)OR^{b}, SCOR^{d}, —NR^{d}SR^{c}, —SR^{b}, —NHS(O)ⱼR^{b}, —COSR^{b}, —C(O)S(O)ⱼR^{b}, —CSR^{b}, —CS(O)₁R^{b}, —C(SO)OH, —C(SO)₂OH, —NR^{d}C(S)R^{c}, —OC(S)R^{b}, —OC(S)OH, —OC(SO)₂R^{b}, —S(O)ⱼNR^{b}R^{c}, —SNR^{b}R^{c}, —S(O)NR^{b}R^{c}, —NR^{d}CS(O)ⱼR^{c}, —C(O)ⱼ(CH₂)ₜNR^{d}(4-10 membered heteroaryl), —C(O)ⱼ(CH₂)ⱼNR^{d}(4-10 membered heterocycloalkyl), —(CR^{d}R^{e})ₜCN, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})_{q}C(O)(CR^{d}R^{e})ₜ(C₃-C₁₀ cycloalkyl), —(CR^{d}R^{e})_{q}C(O)(CR^{d}R^{e})(C₁-C₁₀ aryl), —(CR^{d}R^{e})_{q}C(O)(CR^{d}R^{e})ₜ(4-10 membered heterocycloalkyl), —(CR^{d}R^{e})C(O)(CR^{d}R^{e})(4-10 membered heteroaryl), —(CR^{d}R^{e})ₜO(CR^{d}R^{e})_{q}(C₃-C₁₀ cycloalkyl), —(CR^{d}R^{e})O(CR^{d}R^{e})_{q}(C₆-C₁₀ aryl), —(CR^{d}R^{e})ₜO(CR^{d}R^{e})_{q}(4-10 membered heterocycloalkyl), —(CR^{d}R^{e})ₜO(CR^{d}R^{e})_{q}(4-10 membered heteroaryl), —(CR^{d}R^{e})_{q}SO₂(CR^{d}R^{e})ₜ(C₃-C₁₀ cycloalkyl), —(CR^{d}R^{e})_{q}SO₂(CR^{d}R^{e})ₜ(C₆-C₁₀ aryl), —(CR^{d}R^{e})_{q}SO₂(CR^{d}R^{e})ₜ(4-10 membered heterocycloalkyl), and —(CR^{d}R^{e})_{q}SO₂(CR^{d}R^{e})ₜ(4_{—}10 membered heteroaryl), wherein R^{e} is selected from the group consisting of halo, hydroxyl, —NR^{d}R^{e}, C₁-C₁₀ alkyl, haloalkyl, C₁-C₆ alkoxyl, R^{b} and R^{c} are independently selected from H, C₁-C₁₀ alkyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), and —(CR^{d}R^{e})ₜ(4-10 membered heteroaryl), R^{d} and R^{e} are independently H or C₁-C₆ alkyl, j is an integer from 0 to 2, q and t are each independently an integer from 0 to 5, and 1 or 2 ring carbon atoms of the cyclic moieties of the foregoing R¹⁰ groups are unsubstituted or substituted with ═O, and the alkyl, alkenyl, alkynyl, aryl and cyclic moieties of the foregoing R¹⁰ groups are unsubstituted or substituted with 1 to 3 substituents independently selected from halo, ═O, cyano, nitro, —(CR^{d}R^{e})ₜCN, haloalkyl, 2-10 membered heteroalkyl, —OR^{b}, —C(O)ⱼR^{b}, —NR^{d}C(O)R^{b}, —C(O)NR^{b}R^{c}, —NR^{b}R^{c}, —NR^{b}OR^{c}, —NR^{d}C(O)ⱼNR^{b}R^{c}, —NR^{d}C(O)ⱼR^{b}R^{c}, —OC(O)ⱼR^{b}, —OC(O)NR^{b}R^{c}, —SR^{d}, C₁-C₁₀ alkyl, C₂-C₆ alkenyl, C₂-C₆ alkynyl, —(CR^{d}R^{e})ₜ(C₃-C₁₀ cycloalkyl), —(CR^{d}R^{e})ₜ(C₆-C₁₀ aryl), —(CR^{d}R^{b})ₜ(4-10 membered heterocycloalkyl), —(CR^{d}R^{e})ₜ(4-10 membered heteroaryl), —(CR^{d}R^{e})ₜ(C₆-C₁₀ aryl)-(C₁-C₆ alkyl); and wherein t, R^{b}, R^{c}, R^{d}, R^{e} are as defined above.

5. A compound or pharmaceutically acceptable salt according to claim 4, wherein:
n is 1;
A is —NH—Y— or —O—Y—, wherein Y is C(O);
X is a direct bond, CH₂, or O;
R¹ is a C₆-C₁₀ aryl group unsubstituted or substituted with 1 to 4 R¹⁰ groups;
R² is —P(O)(OR⁴)₂, wherein R⁴ is an H or a C₁-C₁₀ alkyl group that is unsubstituted or substituted with 1 to 4 R¹⁰ groups; and
R³ is a C₆-C₁₀ aryl or 4-10 membered heteroaryl group unsubstituted or substituted with 1 to 4 R¹⁰ groups;
wherein each R¹⁰ is independently selected from halo, amino, ═O, ═S, ═NH, cyano, nitro, hydroxyl, —SH, haloalkyl, 2-10 membered heteroalkyl, C₁-C₆ alkoxy, C₁-C₁₀ alkyl, C₂-C₆ alkenyl, C₂-C₆ alkynyl, —C(O)ⱼR^{a}, OC(O)ⱼR^{d}, —OC(O)OC(O)R^{d}, —OOH, —C(NR^{d})NR^{b}R^{c}, —NR^{d}C(NR^{e})NR^{b}R^{c}, —NR^{d}C(O)ⱼR^{b}, —C(O)NR^{b}R^{c}, —C(O)NR^{d}COR^{b}, —OC(O)NR^{b}R^{c}, —NR^{b}R^{c}, —N R^{d}OR^{c}, —C(S)NR^{b}R^{c}, —NR^{d}C(S)NR^{b}R^{c}, —NR^{d}C(O)NR^{b}R^{c}, —OSH, —S(O)ⱼR^{b}, —OS(O)ⱼR^{b}, —SC(O)R^{b}, —S(O)ⱼC(O)OR^{b}, —SCOR^{d}, —NR^{d}SR^{c}, —SR^{b}, —NHS(O)ⱼR^{b}, —COSR^{b}, —C(O)S(O)ⱼR^{b}, —CSR^{b}, —CS(O)₁R^{b}, —C(SO)OH, —C(SO)₂OH, —NR^{d}C(S)R^{c}, —OC(S)R^{b}, —OC(S)OH, —OC(SO)₂R^{b}, —S(O)ⱼNR^{b}R^{c}, —SNR^{b}R^{c}, —S(O)NR^{b}R^{c}, —NR^{d}CS(O)ⱼR^{c}, —C(O)ⱼ(CH₂)ₜNR^{d}(4-10 membered heteroaryl), —C(O)ⱼ(CH₂)ₜNR^{d}(4-10 membered heterocycloalkyl), —(CR^{d}R^{e})ₜCN, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})ₜC(O)(CR^{d}R^{e})ₜ(C₃-C₁₀ cycloalkyl), —(CR^{d}R^{e})_{q}C(O)(CR^{d}R^{e})ₜ(C₆-C₁₀ aryl), —(CR^{d}R^{e})_{q}C(O)(CR^{d}R^{e})(4-10 membered heterocycloalkyl), —(CR^{d}R^{e})_{q}C(O)(CR^{d}R^{e})ₜ(4-10 membered heteroaryl), —(CR^{d}R^{e})ₜO(CR^{d}R^{e})_{q}(C₃-C₁₀ cycloalkyl), —(CR^{d}R^{e})ₜO(CR^{d}R^{e})_{q}(C₁-C₁₀ aryl), —(CR^{d}R^{e})ₜO(CR^{d}R^{e})ₜ(4-10 membered heterocycloalkyl), —(CR^{d}R^{e})ₜO(CR^{d}R^{e})_{q}(4-10 membered heteroaryl), —(CR^{d}R^{e})_{q}SO₂(CR^{d}R^{e})(C₃-C₁₀ cycloalkyl), —(CR^{d}R^{e})_{q}SO₂(CR^{d}R^{e})ₜ(C₁-C₁₀ aryl), —(CR^{d}R^{e})_{q}SO₂(CR^{d}R^{e})(4-10 membered heterocycloalkyl), and —(CR^{d}R^{e})_{q}SO₂(CR^{d}R^{e})ₜ(4-10 membered heteroaryl), wherein R^{e} is selected from the group consisting of halo, hydroxyl, —NR^{d}R^{e}, C₁-C₁₀ alkyl, haloalkyl, C₁-C₆ alkoxyl, R^{b} and R^{c} are independently selected from H, C₁-C₁₀ alkyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), and —(CR^{d}R^{e})ₜ(4-10 membered heteroaryl), R^{d} and R^{e} are independently H or C₁-C₆ alkyl, j is an integer from 0 to 2, q and t are each independently an integer from 0 to 5, and 1 or 2 ring carbon atoms of the cyclic moieties of the foregoing R¹⁰ groups are unsubstituted or substituted with ═O, and the alkyl, alkenyl, alkynyl, aryl and cyclic moieties of the foregoing R¹⁰ groups are unsubstituted or substituted with 1 to 3 substituents independently selected from halo, ═O, cyano, nitro, —(CR^{d}R^{e})ₜCN, haloalkyl, 2-10 membered heteroalkyl, —OR^{b}, —C(O)ⱼR^{b}, —NR^{d}C(O)R^{b}, —C(O)NR^{b}R^{c}, —NR^{b}R^{c}, —NR OR^{c}, —NR C(O)ⱼNR^{b}R^{c}, —NR^{d}C (O)R^{b}R^{c}, —OC(O)ⱼR^{b}, —OC(O)NR^{b}R^{c}, SR^{d}, C₁-C₁₀ alkyl, C₂-C₆ alkenyl, C₂-C₆ alkynyl, —(CR^{d}R^{e})ₜ(C₃-C₁₀ cycloalkyl), —(CR^{d}R^{e})ₜ(C₆-C₁₀ aryl), —(CR^{d}R^{e})ₜ(4-10 membered heterocycloalkyl), —(CR^{d}R^{e})ₜ(4-10 membered heteroaryl), —(CR^{d}R^{e})ₜ(C₆-C₁₀ aryl)-(C₁-C₆ alkyl); and wherein t, R^{b}, R^{c}, R d R^{e} are as defined above.

6. A compound selected from the group consisting of: 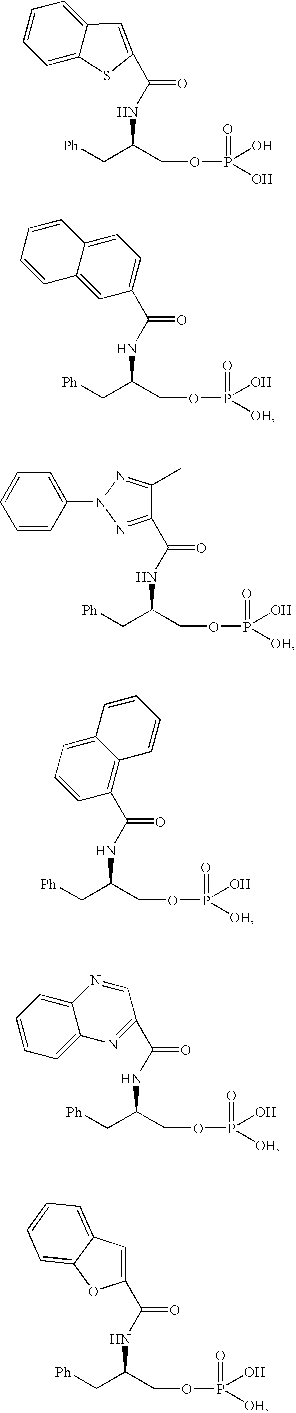 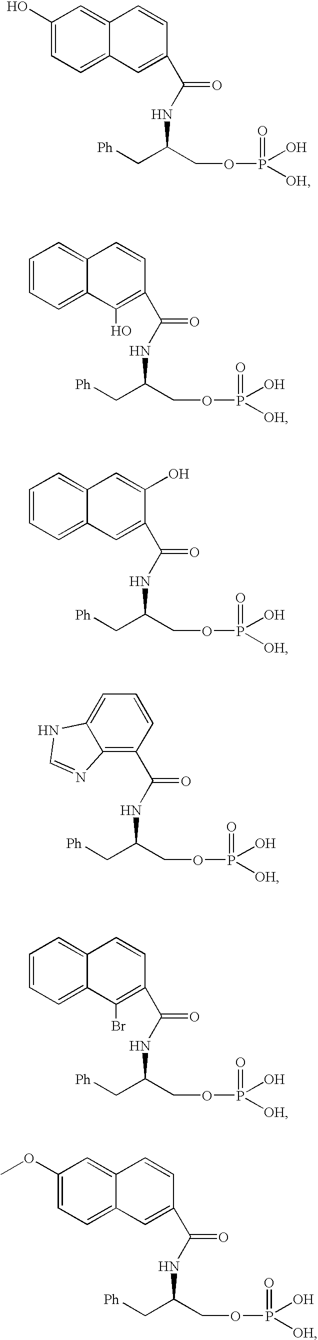 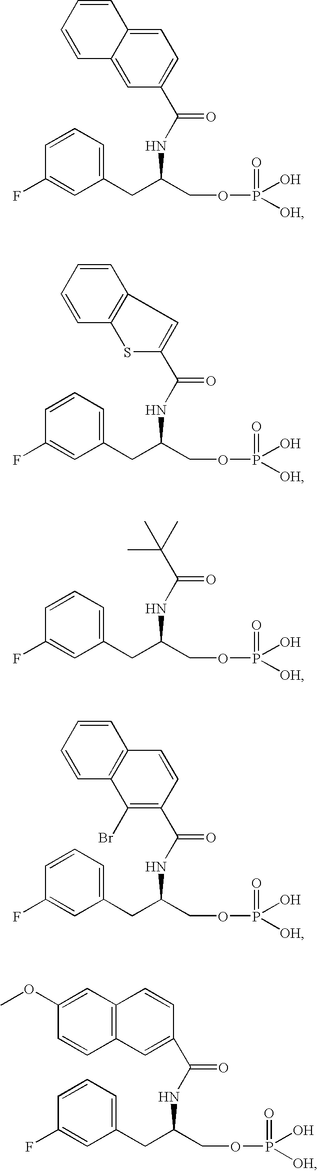 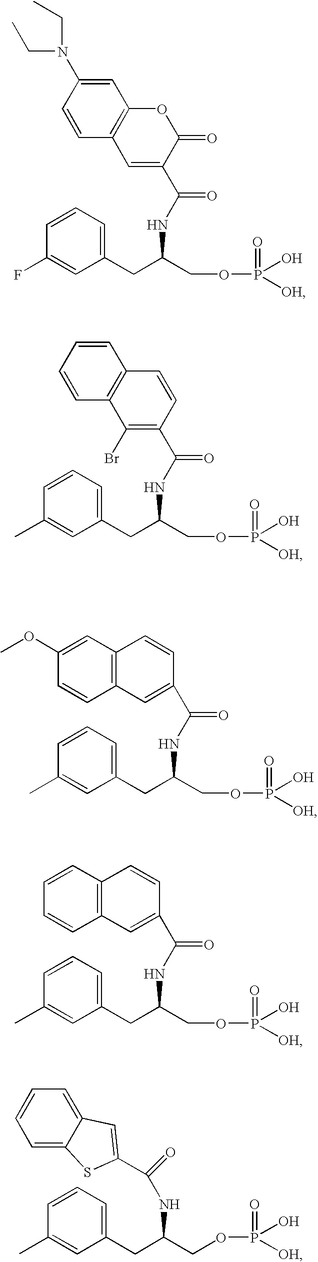 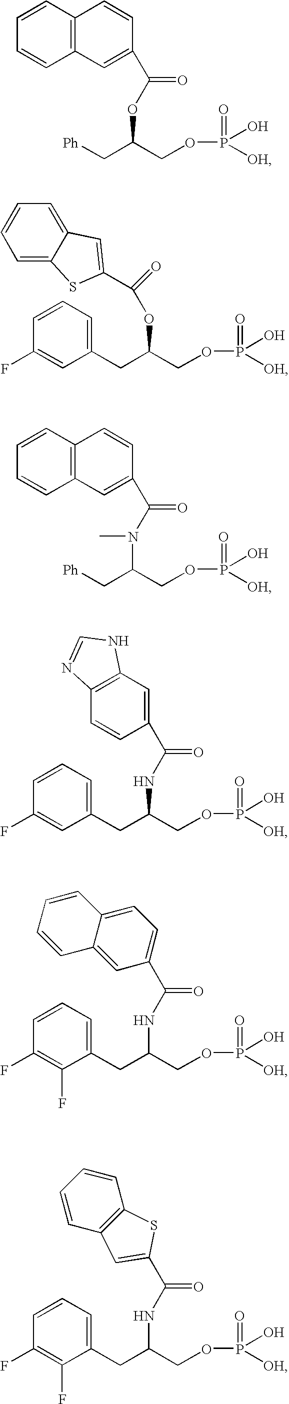 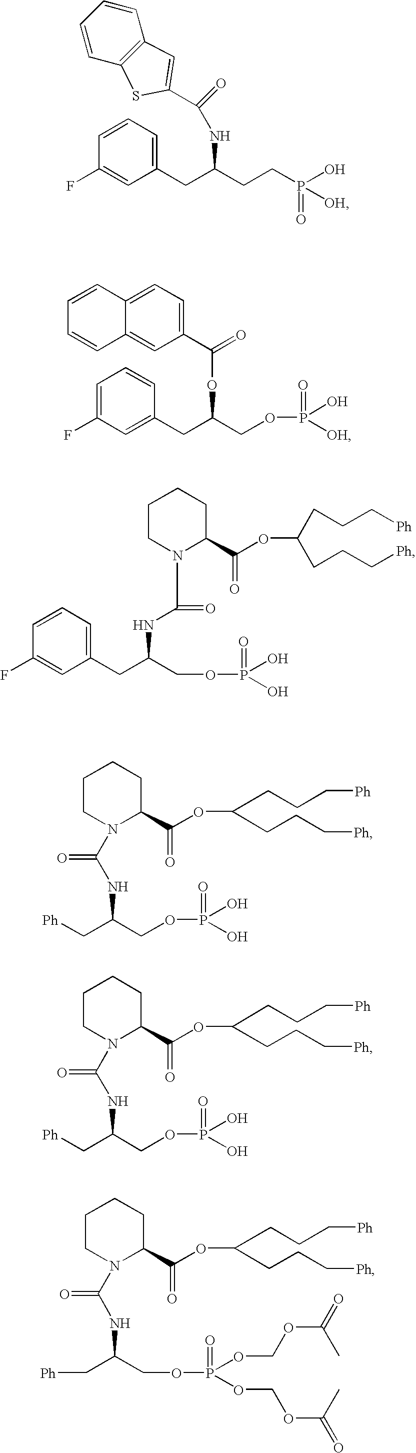 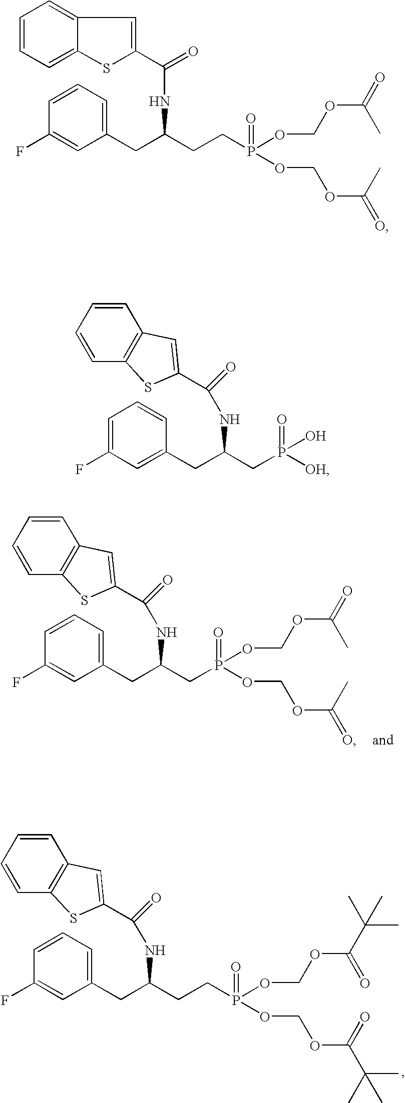
or a pharmaceutically acceptable salt thereof.

7. A pharmaceutical composition comprising: a therapeutically effective amount of an agent selected from the group consisting of compounds, prodrugs, metabolites, and salts as defined in claim 1; and a pharmaceutically acceptable carrier.

8. A method of treating a mammalian disease condition mediated by PIN1 activity, comprising administering to a mammal in need thereof a therapeutically effective amount of a compound, pharmaceutically acceptable prodrug, pharmaceutically active metabolite, or pharmaceutically acceptable salt as defined in claim 1.

9. A method according to claim 8, wherein the mammalian disease condition is associated with hypertension, inappropriate cell proliferation, infectious diseases, or neurodegenerative brain disorders.
